SEQUENCE LISTING (238 sequences)

SEQ ID NO: 1 - DNA, 60 bp, Artificial Sequence
  DNA probe for hepatotoxicity score prediction
agtttgtgataaagtcagaacatgggactccatgtaccaatattgatagaagtttaagac

SEQ ID NO: 2 - DNA, 60 bp, Artificial Sequence
  DNA probe for hepatotoxicity score prediction
gctgtgatcacgagaaaaagtgatcctatgggatccatttcctgtattccatggcagcaa

SEQ ID NO: 3 - DNA, 60 bp, Artificial Sequence
  DNA probe for hepatotoxicity score prediction
aggctggtgatggtgatattagagcggaataaacactcgtgaattggttcacctaaaaaa

SEQ ID NO: 4 - DNA, 60 bp, Artificial Sequence
  DNA probe for hepatotoxicity score prediction
gtaactttaccaaagtgtgcaggtgtatctcgtagaaactgaacatttactgcaggcaga

SEQ ID NO: 5 - DNA, 60 bp, Artificial Sequence
  DNA probe for hepatotoxicity score prediction
aacagtaaatttggggtaaattgttgaaacttgttggtgatgaacttaagcctctattgg

SEQ ID NO: 6 - DNA, 60 bp, Artificial Sequence
  DNA probe for hepatotoxicity score prediction
gtcacacgatgtcatctgaaaactgttatcaggctttgaaaatgtatactaaagaatcac

SEQ ID NO: 7 - DNA, 60 bp, Artificial Sequence
  DNA probe for hepatotoxicity score prediction
gttcacccatgtaaacattcatagaagacatttcctcatgtgttgttgactctttaaaca

SEQ ID NO: 8 - DNA, 60 bp, Artificial Sequence
  DNA probe for hepatotoxicity score prediction
cggtgctttattacaaatgagaacttgagattacctgtagcaattattccattgagacta

SEQ ID NO: 9 - DNA, 60 bp, Artificial Sequence
  DNA probe for hepatotoxicity score prediction
cttgtcactgctgtatataactcccatctgtcagtataataaatatcactgccaacaaaa

SEQ ID NO: 10 - DNA, 60 bp, Artificial Sequence
  DNA probe for hepatotoxicity score prediction
attgatggttctttgattcaagtcaggtgtagtgagtatgctttcatgtcgtcattatgt

SEQ ID NO: 11 - DNA, 60 bp, Artificial Sequence
  DNA probe for hepatotoxicity score prediction
caatgttaccagatggatactctcttgtgctacctctgagcttgctatagacacaaaggc

SEQ ID NO: 12 - DNA, 60 bp, Artificial Sequence
  DNA probe for hepatotoxicity score prediction
catagcagtcaccaaatctagtaaatccatagaaagagagagatatttcctaaaaccatg

SEQ ID NO: 13 - DNA, 60 bp, Artificial Sequence
  DNA probe for hepatotoxicity score prediction
caccggattcaaaccaatcaactgtgaattgtgaaactgaagttgttctttcggttttta

SEQ ID NO: 14 - DNA, 60 bp, Artificial Sequence
  DNA probe for hepatotoxicity score prediction
caagttttgattatgctataggttgatttttgtgttaatccaaattggaatagctattga

SEQ ID NO: 15 - DNA, 60 bp, Artificial Sequence
  DNA probe for hepatotoxicity score prediction
aatccaaccaacaaaactggaaagcccaacatgtcataaagtttctcctccacctccgtg

SEQ ID NO: 16 - DNA, 60 bp, Artificial Sequence
  DNA probe for hepatotoxicity score prediction
aggcaacacatggttcaattctgtcactgtaactggacaaccagaagatttgtcaagtcc

SEQ ID NO: 17 - DNA, 60 bp, Artificial Sequence
  DNA probe for hepatotoxicity score prediction
tacagatgctctacctggtaactctttaggattagacttgatcaccacaagaattatttt

SEQ ID NO: 18 - DNA, 60 bp, Artificial Sequence
  DNA probe for hepatotoxicity score prediction
cacatttgaaacagtggatgatagctggaatgttgaagatctctgtaaataaagctttac

SEQ ID NO: 19 - DNA, 60 bp, Artificial Sequence
  DNA probe for hepatotoxicity score prediction
tttgtaatttaaggtctgttcacagctgtaattattattttctacaataaatggcacctg

SEQ ID NO: 20 - DNA, 60 bp, Artificial Sequence
  DNA probe for hepatotoxicity score prediction
aatttagagaaagttgaaggtaggagcttttaggatgtgggagcaaaacttcacagggaa

SEQ ID NO: 21 - DNA, 60 bp, Artificial Sequence
  DNA probe for hepatotoxicity score prediction
gtgaatacttgaaaaatgtacaaatctttcatccatacctgtgcatgagctgtattcttc

SEQ ID NO: 22 - DNA, 60 bp, Artificial Sequence
  DNA probe for hepatotoxicity score prediction
gacggtggtgatctgagagtgaagttggaccttctgtaaacatgtggagtttggtttctt

SEQ ID NO: 23 - DNA, 60 bp, Artificial Sequence
  DNA probe for hepatotoxicity score prediction
tgccattgttcaagtggatatcaatgttctctgtcaggtattcccagacctctttggagg

SEQ ID NO: 24 - DNA, 60 bp, Artificial Sequence
  DNA probe for hepatotoxicity score prediction
gaattggagatgaactggacaacaacatggagctgcagaggatgattgctgatgtggata

SEQ ID NO: 25 - DNA, 60 bp, Artificial Sequence
  DNA probe for hepatotoxicity score prediction
aactgagaaaaggattgcagataagaagataaagacttctgtgatagcgatcagatgttg

SEQ ID NO: 26 - DNA, 60 bp, Artificial Sequence
  DNA probe for hepatotoxicity score prediction
acatcctgaagctgtcttatctatggtagaagctgtgtgactacctccaaacttggtttt

SEQ ID NO: 27 - DNA, 60 bp, Artificial Sequence
  DNA probe for hepatotoxicity score prediction
gggagcgcttcgatccgttatatgttctgtgtttaaagaagaaaacctatttattaatga

SEQ ID NO: 28 - DNA, 60 bp, Artificial Sequence
  DNA probe for hepatotoxicity score prediction
acttataattggaaattgccttcccactcagtgtgagtttctgtcaacagtacagtgttg

SEQ ID NO: 29 - DNA, 60 bp, Artificial Sequence
  DNA probe for hepatotoxicity score prediction
gcctgatgactacttggtgactgattcctgaacaagagaagaattttagcttcaagcctt

SEQ ID NO: 30 - DNA, 60 bp, Artificial Sequence
  DNA probe for hepatotoxicity score prediction
ctacggaactggtttcgttttgcttaaggggaaaatgcacttttgtttggcaattcaaaa

SEQ ID NO: 31 - DNA, 60 bp, Artificial Sequence
  DNA probe for hepatotoxicity score prediction
gcctatttaaaaagccagctaatagttcagaagattgtctattttcatacagcgagaacg

SEQ ID NO: 32 - DNA, 60 bp, Artificial Sequence
  DNA probe for hepatotoxicity score prediction
caacccccaacttcattgtaaacccaaacatgatgaaaaattctagattttatgtcttcc

SEQ ID NO: 33 - DNA, 60 bp, Artificial Sequence
  DNA probe for hepatotoxicity score prediction
gggaatgtatggctctacttaattgtaaccagtattctaccgaatgaataaagatctttg

SEQ ID NO: 34 - DNA, 60 bp, Artificial Sequence
  DNA probe for hepatotoxicity score prediction
tcagaaggagatttacggaaagcaattacatttcttcaaagtgctactcgactaacaggt

SEQ ID NO: 35 - DNA, 60 bp, Artificial Sequence
  DNA probe for hepatotoxicity score prediction
acctcagcaatggcacagtacagattaatttcttccaggaccacaccaaacttatcctgt

SEQ ID NO: 36 - DNA, 60 bp, Artificial Sequence
  DNA probe for hepatotoxicity score prediction
tatttattaagcatcacacttcccgggggttgaactttttgcaaactgtgcaattggtaa

SEQ ID NO: 37 - DNA, 60 bp, Artificial Sequence
  DNA probe for hepatotoxicity score prediction
gtattttagtagcaaaagtttaagtacgaatatatatatatattttagaagttattttgc

SEQ ID NO: 38 - DNA, 60 bp, Artificial Sequence
  DNA probe for hepatotoxicity score prediction
cagaagcaaaataaactttgtgtgctagttttgatgatgaaaggtagaatttctacctgg

SEQ ID NO: 39 - DNA, 60 bp, Artificial Sequence
  DNA probe for hepatotoxicity score prediction
acggtcgttcgtagacaacatggtgtctgttactgttttttagaagattcaggaacacca

SEQ ID NO: 40 - DNA, 60 bp, Artificial Sequence
  DNA probe for hepatotoxicity score prediction
gaagtctgtgtcacagtaacagaattgccacaacagtcccatcaagttgagtaaagcttt

SEQ ID NO: 41 - DNA, 60 bp, Artificial Sequence
  DNA probe for hepatotoxicity score prediction
gatgttgcaagtcactgagcaatgtacacccttaatgtataagtgcttttatttgtacag

SEQ ID NO: 42 - DNA, 60 bp, Artificial Sequence
  DNA probe for hepatotoxicity score prediction
aggcatttggaacaatggattgatgctctttatgatactattcaaattattggagaggag

SEQ ID NO: 43 - DNA, 60 bp, Artificial Sequence
  DNA probe for hepatotoxicity score prediction
aaataagggacatgatcactgaaaataaagtaagggtttgtaatagaatgttctctcggg

SEQ ID NO: 44 - DNA, 60 bp, Artificial Sequence
  DNA probe for hepatotoxicity score prediction
tacagacaagatgtaattgtgtgtgttcttcctaaaattgtatcgcctctaaagttgccc

SEQ ID NO: 45 - DNA, 60 bp, Artificial Sequence
  DNA probe for hepatotoxicity score prediction
ctcagccaaccaagtagaatcacgtatttattttccatttgtacatccaagagtatgaat

SEQ ID NO: 46 - DNA, 60 bp, Artificial Sequence
  DNA probe for hepatotoxicity score prediction
tagtattgttgaggatctaccattttaccacgcgtgctttgtcttacagggactgaatga

SEQ ID NO: 47 - DNA, 60 bp, Artificial Sequence
  DNA probe for hepatotoxicity score prediction
ggctcctggataaatttcttcatgcactaatgccatccactttcatccctctctatacca

SEQ ID NO: 48 - DNA, 60 bp, Artificial Sequence
  DNA probe for hepatotoxicity score prediction
gtccgagtaaaaatctaaattatatggagtgaaaatcgttctctacatggatttctccga

SEQ ID NO: 49 - DNA, 60 bp, Artificial Sequence
  DNA probe for hepatotoxicity score prediction
ggctttcctgaaagttctcaaggagcttgagttaatgagtcagattttcttattaaaggg

SEQ ID NO: 50 - DNA, 60 bp, Artificial Sequence
  DNA probe for hepatotoxicity score prediction
ctgaagattttgtctgacacttacagtgccgagaaagtggaagtacatcgcctcattagg

SEQ ID NO: 51 - DNA, 60 bp, Artificial Sequence
  DNA probe for hepatotoxicity score prediction
gtagaatgacaagtacttaacccatgtttggtgttttaaagtgaaacaaaatcagaccag

SEQ ID NO: 52 - DNA, 60 bp, Artificial Sequence
  DNA probe for hepatotoxicity score prediction
actactgtatcaaagggtttttgctatgtggacttcacttcaataaaaaacagttgcttg

SEQ ID NO: 53 - DNA, 60 bp, Artificial Sequence
  DNA probe for hepatotoxicity score prediction
cacttttaccctcagtatcatggctctgaactaaaattaccattcggattttcccacaga

SEQ ID NO: 54 - DNA, 60 bp, Artificial Sequence
  DNA probe for hepatotoxicity score prediction
gacagctttcagtactgagatctgtaaaattgtgggtatggaattaaagtgacttgtttg

SEQ ID NO: 55 - DNA, 60 bp, Artificial Sequence
  DNA probe for hepatotoxicity score prediction
taaccccattcctaacagtgttcagtacattatggtttgactcgttcggaatatattaag

SEQ ID NO: 56 - DNA, 60 bp, Artificial Sequence
  DNA probe for hepatotoxicity score prediction
tcagttcgtcgttagcgcgtaggtcatctcctccaccgccgttcaactgcggcatttttt

SEQ ID NO: 57 - DNA, 60 bp, Artificial Sequence
  DNA probe for hepatotoxicity score prediction
caacacattaaatgattctttaaaacctggtctgtgtcagtatgctgtctactcacacac

SEQ ID NO: 58 - DNA, 60 bp, Artificial Sequence
  DNA probe for hepatotoxicity score prediction
actcagaaagcccaatttgttttccacagtatcgggaagtattttatctaaaatggtggt

SEQ ID NO: 59 - DNA, 60 bp, Artificial Sequence
  DNA probe for hepatotoxicity score prediction
aaacagactaaatattgccctaaacatcaacagaggttgaatcatttagctaaacgtcct

SEQ ID NO: 60 - DNA, 60 bp, Artificial Sequence
  DNA probe for hepatotoxicity score prediction
ctgtgtatgtgtcgtgagaaggggattagtgattggaaactatccctaaccccagttcta

SEQ ID NO: 61 - DNA, 60 bp, Artificial Sequence
  DNA probe for hepatotoxicity score prediction
cctagaacttaaaaaccaagtttcacagtgtatatatgtgtgtatatgctgccactaatc

SEQ ID NO: 62 - DNA, 60 bp, Artificial Sequence
  DNA probe for hepatotoxicity score prediction
gggaatcgattcagagccagacttcccaaaggcttaagaccttaggcaacatcaataaag

SEQ ID NO: 63 - DNA, 60 bp, Artificial Sequence
  DNA probe for hepatotoxicity score prediction
gaaccttctgtttaggttctggtgatcaagaattatgtagtaaaacatagctgatattgc

SEQ ID NO: 64 - DNA, 60 bp, Artificial Sequence
  DNA probe for hepatotoxicity score prediction
aaactctgaagaaggatgaagacattgtatacaccaagatattaagaacaagaagtcccc

SEQ ID NO: 65 - DNA, 60 bp, Artificial Sequence
  DNA probe for hepatotoxicity score prediction
aaagtggtctgagagtggatacactgcacaaggtaggccatatctcatttgagtttgtgg

SEQ ID NO: 66 - DNA, 60 bp, Artificial Sequence
  DNA probe for hepatotoxicity score prediction
ctcatcattgataacttcatacctcaggactaccaggaaatgattgaaaactacgtccac

SEQ ID NO: 67 - DNA, 60 bp, Artificial Sequence
  DNA probe for hepatotoxicity score prediction
gttttgtgagaatgaccttaactaagctttgatatgacagccatagaatgaaatgcagag

SEQ ID NO: 68 - DNA, 60 bp, Artificial Sequence
  DNA probe for hepatotoxicity score prediction
cttttagattaaagagcaacttagaagtgtttgcacacttttccagaacgttcttggagc

SEQ ID NO: 69 - DNA, 60 bp, Artificial Sequence
  DNA probe for hepatotoxicity score prediction
tcaaaagtaaacccaatctgctagaacattctgaaagtgatactattgggtctgattttg

SEQ ID NO: 70 - DNA, 60 bp, Artificial Sequence
  DNA probe for hepatotoxicity score prediction
ggtgatactgtagaaaccctgtaggatatttaaaatattaaacgtgaaccaaggttttgc

SEQ ID NO: 71 - DNA, 60 bp, Artificial Sequence
  DNA probe for hepatotoxicity score prediction
aaccagacctagattcatacgtgtttctgagagtgaaagaacgacaggaaaacatactag

SEQ ID NO: 72 - DNA, 60 bp, Artificial Sequence
  DNA probe for hepatotoxicity score prediction
cacaaaagggcagtgctgtacatgttgcttcaataaataaaaggagtctgtcgtaaaaaa

SEQ ID NO: 73 - DNA, 60 bp, Artificial Sequence
  DNA probe for hepatotoxicity score prediction
cattttttaagacaaggaagtttcccatcagcgaatgaacatctgtgactagtgccgaag

SEQ ID NO: 74 - DNA, 60 bp, Artificial Sequence
  DNA probe for hepatotoxicity score prediction
atgagggatgagatgttctgaaggtgtttccaattaggaaacgtaatcttaacccccatg

SEQ ID NO: 75 - DNA, 60 bp, Artificial Sequence
  DNA probe for hepatotoxicity score prediction
agcccagtttcatgtgtgaatcacttctcttgtaaacatggataaagtaaaacgtgtgtg

SEQ ID NO: 76 - DNA, 60 bp, Artificial Sequence
  DNA probe for hepatotoxicity score prediction
gttactgatgcaaatggagtctgaaaggcagtattccactatataaacattttctatagg

SEQ ID NO: 77 - DNA, 60 bp, Artificial Sequence
  DNA probe for hepatotoxicity score prediction
cagaccgttggtttacccaggatcattcctttggttagtaaataaatgcgtttgtgctaa

SEQ ID NO: 78 - DNA, 60 bp, Artificial Sequence
  DNA probe for hepatotoxicity score prediction
gaatcacagggaacagaccatcatcagaagagaacgttataatttaaacctatttgctgt

SEQ ID NO: 79 - DNA, 60 bp, Artificial Sequence
  DNA probe for hepatotoxicity score prediction
gtggactccatatgaagatttggagaagcaggatgctaaaatcagcatgatggacaagtt

SEQ ID NO: 80 - DNA, 60 bp, Artificial Sequence
  DNA probe for hepatotoxicity score prediction
ggtcagtttgcgtagtcacccacctatgtttacattttacagaaattactgctctgtaac

SEQ ID NO: 81 - DNA, 60 bp, Artificial Sequence
  DNA probe for hepatotoxicity score prediction
tcaccgaggaacactgatataacccattggttttttctgtgtttgataggtaataaaaac

SEQ ID NO: 82 - DNA, 60 bp, Artificial Sequence
  DNA probe for hepatotoxicity score prediction
ggcaagtgtaacttgataccattttctcaactctttataaccctgccaactttcaaaata

SEQ ID NO: 83 - DNA, 60 bp, Artificial Sequence
  DNA probe for hepatotoxicity score prediction
catggctgttctatacaaaattggtagttgattatggatgcaaatcactgaggaatgtta

SEQ ID NO: 84 - DNA, 60 bp, Artificial Sequence
  DNA probe for hepatotoxicity score prediction
tccatggtgaaaagtttaattcagcagctactaaggctctcctatatgctggttcaggtt

SEQ ID NO: 85 - DNA, 60 bp, Artificial Sequence
  DNA probe for hepatotoxicity score prediction
gcagaagtctaacgtgctcagtacgctgttttaatatttacatgccattttaataaaacg

SEQ ID NO: 86 - DNA, 60 bp, Artificial Sequence
  DNA probe for hepatotoxicity score prediction
caggactaaccgcgtttacaggatctgagtgttgaactgatgatatctgtacgtcacctt

SEQ ID NO: 87 - DNA, 60 bp, Artificial Sequence
  DNA probe for hepatotoxicity score prediction
aattggattgctggctttgaaattagggtcatcattagatcatttgtaaaactaggtacc

SEQ ID NO: 88 - DNA, 60 bp, Artificial Sequence
  DNA probe for hepatotoxicity score prediction
gtgctctggagataatgatgtaaaactatctagaggctgaaagtatggacttacggaatt

SEQ ID NO: 89 - DNA, 60 bp, Artificial Sequence
  DNA probe for hepatotoxicity score prediction
aatttactgcaagcataacatggagttcttgttacactagaaagtggattacaaatctcc

SEQ ID NO: 90 - DNA, 60 bp, Artificial Sequence
  DNA probe for hepatotoxicity score prediction
aggaacaattggtgatttttatgcagaaactaaataatccttaataaataaatctatatt

SEQ ID NO: 91 - DNA, 60 bp, Artificial Sequence
  DNA probe for hepatotoxicity score prediction
ctccttttaggaacagaacatgtacacatttacttgtattctgaaaatataatggtgccg

SEQ ID NO: 92 - DNA, 60 bp, Artificial Sequence
  DNA probe for hepatotoxicity score prediction
cactctctagaatgaagattcactgaagaagtgtgttgaggattacatagtgaagattgg

SEQ ID NO: 93 - DNA, 60 bp, Artificial Sequence
  DNA probe for hepatotoxicity score prediction
tgacccgaataattaaccatctcctaaaattctcctgaaacatacagtaggaactaaatg

SEQ ID NO: 94 - DNA, 60 bp, Artificial Sequence
  DNA probe for hepatotoxicity score prediction
gttatctacatgtatatacatatatgtgtttataatgtgtatctgatcatggaaataaat

SEQ ID NO: 95 - DNA, 60 bp, Artificial Sequence
  DNA probe for hepatotoxicity score prediction
atcagtctgtgtacgttgcatgttaatttccaggtggaattgtttgagatggaaatgcag

SEQ ID NO: 96 - DNA, 60 bp, Artificial Sequence
  DNA probe for hepatotoxicity score prediction
agcacttatgggcgtgattgtgtatcacatatacatatagaccagcacacccagagtctc

SEQ ID NO: 97 - DNA, 60 bp, Artificial Sequence
  DNA probe for hepatotoxicity score prediction
tgggatacaaatactcaactctacaatgaaacacttgaatgattcttgaaagacacagtg

SEQ ID NO: 98 - DNA, 60 bp, Artificial Sequence
  DNA probe for hepatotoxicity score prediction
cccttcttctgtatcaggttattggttgtacatataaattatactttcctttctgtgtgc

SEQ ID NO: 99 - DNA, 60 bp, Artificial Sequence
  DNA probe for hepatotoxicity score prediction
gataaccgagaagcagaagatactgaataacacttaacaagtctctctgaaggacatctg

SEQ ID NO: 100 - DNA, 60 bp, Artificial Sequence
  DNA probe for hepatotoxicity score prediction
tgattttatatagttctgtgttttaatgtatctgtgtatatacatatatatggaaaatgt

SEQ ID NO: 101 - DNA, 60 bp, Artificial Sequence
  DNA probe for hepatotoxicity score prediction
cattgctcctaggtcttcccaggtaccttgtagaagaacttaaatctataaaataaggct

SEQ ID NO: 102 - DNA, 60 bp, Artificial Sequence
  DNA probe for hepatotoxicity score prediction
tagatgcacaggagtaaccttggatatatgcttgctcaataaagatgtagggagtatgaa

SEQ ID NO: 103 - DNA, 60 bp, Artificial Sequence
  DNA probe for hepatotoxicity score prediction
tgttgtgtgatgtacagggcaccattatatctacacgaatgtggttataaacttgagtaa

SEQ ID NO: 104 - DNA, 60 bp, Artificial Sequence
  DNA probe for hepatotoxicity score prediction
gtggtttatggatttatatttttaattataacaagatattcaggataggtataaactcag

SEQ ID NO: 105 - DNA, 60 bp, Artificial Sequence
  DNA probe for hepatotoxicity score prediction
acattgctcagcttctacagatcctttcttttataagatgcatgccaaacgtgttccact

SEQ ID NO: 106 - DNA, 60 bp, Artificial Sequence
  DNA probe for hepatotoxicity score prediction
gtcaggacatcagccttgcaatgtgagccctgtaaataaaacatgaatttttaccatctg

SEQ ID NO: 107 - DNA, 60 bp, Artificial Sequence
  DNA probe for hepatotoxicity score prediction
gatcatagagaaggttgttcatcgtctcacacactacgatcacgttctgattgagctcac

SEQ ID NO: 108 - DNA, 60 bp, Artificial Sequence
  DNA probe for hepatotoxicity score prediction
cccaagaatgtgatgtacgcagacatgctcatgtgaaaccagtttctgtacttttgggag

SEQ ID NO: 109 - DNA, 60 bp, Artificial Sequence
  DNA probe for hepatotoxicity score prediction
tgcttctgaaaagtactctgaaaaggaggataaatatgaagaagaaatcaagcttctgtc

SEQ ID NO: 110 - DNA, 60 bp, Artificial Sequence
  DNA probe for hepatotoxicity score prediction
ctgtggaagggatggttttctgttctgtggagtgtaagaagatgatgtcctgagtgaaca

SEQ ID NO: 111 - DNA, 60 bp, Artificial Sequence
  DNA probe for hepatotoxicity score prediction
cttttagtgtcaaaataaagatttgtatagactgtttttaagggacacccattcaaggcc

SEQ ID NO: 112 - DNA, 60 bp, Artificial Sequence
  DNA probe for hepatotoxicity score prediction
ggtaaacaccttcatgaggaacctgtaagcaataggtagacatcttgactgtagtctggg

SEQ ID NO: 113 - DNA, 60 bp, Artificial Sequence
  DNA probe for hepatotoxicity score prediction
acaaacaagaagagaagcaagtgctgttacaggaaagcagtctgacaccatgggagatgt

SEQ ID NO: 114 - DNA, 60 bp, Artificial Sequence
  DNA probe for hepatotoxicity score prediction
aagctgaattactacccttcgctcactaaaatgatggtgaatttgtttttagtacaggga

SEQ ID NO: 115 - DNA, 60 bp, Artificial Sequence
  DNA probe for hepatotoxicity score prediction
gggcatacacaagcaggctgcactacattttgtaaatttttattaaaaagaaaggaccgt

SEQ ID NO: 116 - DNA, 60 bp, Artificial Sequence
  DNA probe for hepatotoxicity score prediction
gggtctcaaacaaggctttgtgtgtgcttgacaggcactcactctaaaaactgtgttaca

SEQ ID NO: 117 - DNA, 60 bp, Artificial Sequence
  DNA probe for hepatotoxicity score prediction
aaacaatatttgatcgactgtgtacacagctagaaaagatcggacagcaaattaacagag

SEQ ID NO: 118 - DNA, 60 bp, Artificial Sequence
  DNA probe for hepatotoxicity score prediction
tcatccataggttacagcagagctaatttatcagacgtaaggaaagagacagttcctgtc

SEQ ID NO: 119 - DNA, 60 bp, Artificial Sequence
  DNA probe for hepatotoxicity score prediction
aaagaaacttgatgacacaattcagcatatctatgagactctgttgtctatcccagaggt

SEQ ID NO: 120 - DNA, 60 bp, Artificial Sequence
  DNA probe for hepatotoxicity score prediction
tgaaccatattccccacataagctacaaaatgagctacccactacaaataagaacctttc

SEQ ID NO: 121 - DNA, 60 bp, Artificial Sequence
  DNA probe for hepatotoxicity score prediction
gccaggagaagaatccttaatttgcagtactgtttctctatagtgtaaaggtcattttaa

SEQ ID NO: 122 - DNA, 60 bp, Artificial Sequence
  DNA probe for hepatotoxicity score prediction
cagcaaaggaaactaaaagcatttggttggagaagatctcaacaaatttctggaagagag

SEQ ID NO: 123 - DNA, 60 bp, Artificial Sequence
  DNA probe for hepatotoxicity score prediction
ctgagtggtatttcaaagacatataaagttccagaattcttgctacactttaaagcttgc

SEQ ID NO: 124 - DNA, 60 bp, Artificial Sequence
  DNA probe for hepatotoxicity score prediction
atctgctgatttatactgacaaagatttggtggtacctgaaaagtgggaagaatcgggac

SEQ ID NO: 125 - DNA, 60 bp, Artificial Sequence
  DNA probe for hepatotoxicity score prediction
cactaagtacatgcctcgctcaagagtttagattttagcatagtttaaagttgagctgcc

SEQ ID NO: 126 - DNA, 60 bp, Artificial Sequence
  DNA probe for hepatotoxicity score prediction
gtgtcgtggcttggaatgttattattgccgaggacctggttagaggtataaagacctttt

SEQ ID NO: 127 - DNA, 60 bp, Artificial Sequence
  DNA probe for hepatotoxicity score prediction
caaggtcttataaactgttggtcaaaacagttgtgtttattacactgtcactgatacagt

SEQ ID NO: 128 - DNA, 60 bp, Artificial Sequence
  DNA probe for hepatotoxicity score prediction
gagctgcccattaaaacctgcttgaagtgagcacgtcataaaataaaagtgtgtccctgt

SEQ ID NO: 129 - DNA, 60 bp, Artificial Sequence
  DNA probe for hepatotoxicity score prediction
tcaaagccagtctttaaacatccatatcgctgagcccaatatggcaaactcactagtatg

SEQ ID NO: 130 - DNA, 60 bp, Artificial Sequence
  DNA probe for hepatotoxicity score prediction
tttctggcaaccctggtcttgatctctaaggatgtgtcataaatggactgtaccatatag

SEQ ID NO: 131 - DNA, 60 bp, Artificial Sequence
  DNA probe for hepatotoxicity score prediction
gaaaaacatagatgagaaactcacagaagcagccagaaaactaggatactcactggagca

SEQ ID NO: 132 - DNA, 60 bp, Artificial Sequence
  DNA probe for hepatotoxicity score prediction
aggagaacttggagctgagaaacaaatatgaggacctcaacacgaatatctggagatggg

SEQ ID NO: 133 - DNA, 60 bp, Artificial Sequence
  DNA probe for hepatotoxicity score prediction
atgttagctatttcactttactaactactttccgaaagaagcgtaatcagaaaacgtacc

SEQ ID NO: 134 - DNA, 60 bp, Artificial Sequence
  DNA probe for hepatotoxicity score prediction
ccctgagccttcctgaagaaccagtgtagacattcttctttcagggtgcaaggcccctgg

SEQ ID NO: 135 - DNA, 60 bp, Artificial Sequence
  DNA probe for hepatotoxicity score prediction
agctggttacatgaaattaatagtcaagaattagaaaaggctcatacaacactgctaggt

SEQ ID NO: 136 - DNA, 60 bp, Artificial Sequence
  DNA probe for hepatotoxicity score prediction
tagaaagattcctggtgataagctgcagtttatatttactagtattgaccctaagaatcc

SEQ ID NO: 137 - DNA, 60 bp, Artificial Sequence
  DNA probe for hepatotoxicity score prediction
tggcattcaaaccatagtctggacaaaggcaccaataagtaagatttctaggccaactcc

SEQ ID NO: 138 - DNA, 60 bp, Artificial Sequence
  DNA probe for hepatotoxicity score prediction
cctgagcgtgacttgtcagtatgaggagagattcaagatgaataagaaatactggtgcag

SEQ ID NO: 139 - DNA, 60 bp, Artificial Sequence
  DNA probe for hepatotoxicity score prediction
taacagtgagtttactaccaaccgtcaaagtaacttaaaggaaacaataaaacaccatcg

SEQ ID NO: 140 - DNA, 60 bp, Artificial Sequence
  DNA probe for hepatotoxicity score prediction
gtccagctcttccgatgagggcagcccctgccttgctgtaggaaggacactgggatgcag

SEQ ID NO: 141 - DNA, 60 bp, Artificial Sequence
  DNA probe for hepatotoxicity score prediction
tgtgggtggtagggcataccatggctcaaatgtggaaaccaaagaaaagcttttggaagt

SEQ ID NO: 142 - DNA, 60 bp, Artificial Sequence
  DNA probe for hepatotoxicity score prediction
ctcacaagacttctcgtgacattgaagtgcagggcttccttatccctaaggggacaaccc

SEQ ID NO: 143 - DNA, 60 bp, Artificial Sequence
  DNA probe for hepatotoxicity score prediction
catgattgctggaaatggatacacaactattgtcccagacttctttgtgggtcaagagcc

SEQ ID NO: 144 - DNA, 60 bp, Artificial Sequence
  DNA probe for hepatotoxicity score prediction
cagacgggattctggtcaacaaggagttgtgaatttctcgtacgatgatttcatccagtg

SEQ ID NO: 145 - DNA, 60 bp, Artificial Sequence
  DNA probe for hepatotoxicity score prediction
ggagcccccagctttgtaatcatgtgatcagtctgaatcattgtttctgtcacctgactt

SEQ ID NO: 146 - DNA, 60 bp, Artificial Sequence
  DNA probe for hepatotoxicity score prediction
ttacctgttctagaataagtaatcatcactactgtaccaccttgaaaatactgtttccag

SEQ ID NO: 147 - DNA, 60 bp, Artificial Sequence
  DNA probe for hepatotoxicity score prediction
gatgactacgctacggaggatgggacaggcgtgagggattacattcatgtggtggatctg

SEQ ID NO: 148 - DNA, 60 bp, Artificial Sequence
  DNA probe for hepatotoxicity score prediction
agtcaagtgttgatgaatctccattagatgaaaaagggaaatttctattggatactgtgg

SEQ ID NO: 149 - DNA, 60 bp, Artificial Sequence
  DNA probe for hepatotoxicity score prediction
gggttgtccactgtcttaggaagacctattttaaccttacgtgttcaatgtggtgaatgg

SEQ ID NO: 150 - DNA, 60 bp, Artificial Sequence
  DNA probe for hepatotoxicity score prediction
aaacaaagcacttgggtttaacaagacgcattgctaatttctggttttgtacagaccagc

SEQ ID NO: 151 - DNA, 60 bp, Artificial Sequence
  DNA probe for hepatotoxicity score prediction
atcttggacaaggttttcctgatataacccttccttcatatgtacaagaagaattgtcaa

SEQ ID NO: 152 - DNA, 60 bp, Artificial Sequence
  DNA probe for hepatotoxicity score prediction
gaggatgtagggttggaatccaaccaaagtgttgacccttaacatattttgcttatcacc

SEQ ID NO: 153 - DNA, 60 bp, Artificial Sequence
  DNA probe for hepatotoxicity score prediction
agacttcaatgtttcctttcccttagaattctctggaattcagctgctttagaagtctca

SEQ ID NO: 154 - DNA, 60 bp, Artificial Sequence
  DNA probe for hepatotoxicity score prediction
cccctctggatttaaacaggagtggactgttctggctcctgtctttagagcaaagcagct

SEQ ID NO: 155 - DNA, 60 bp, Artificial Sequence
  DNA probe for hepatotoxicity score prediction
tccaagtgctaagtgttgagtgttcatccagagacaggacgtctgtatcattccctgaaa

SEQ ID NO: 156 - DNA, 60 bp, Artificial Sequence
  DNA probe for hepatotoxicity score prediction
agtttccgttcacacaagcttgaggtctttccaagagtgtttgaccacctacttggacac

SEQ ID NO: 157 - DNA, 60 bp, Artificial Sequence
  DNA probe for hepatotoxicity score prediction
cctccaacttttctgtcagatctgagaccttacaaaaagtaatcaagaactgcactgact

SEQ ID NO: 158 - DNA, 60 bp, Artificial Sequence
  DNA probe for hepatotoxicity score prediction
ggagccttttaagagattcatttgccagaataaaaatgtaagcaccccttaggtttctct

SEQ ID NO: 159 - DNA, 60 bp, Artificial Sequence
  DNA probe for hepatotoxicity score prediction
cacaccgtgctgccttaattgaatacataatatggaacttgatgacaatccattcataat

SEQ ID NO: 160 - DNA, 60 bp, Artificial Sequence
  DNA probe for hepatotoxicity score prediction
ccattaagtttcttgaagaatgaagggattatcacatcgccctgttataattatggagta

SEQ ID NO: 161 - DNA, 60 bp, Artificial Sequence
  DNA probe for hepatotoxicity score prediction
ggcagttggtatacgtgggtacttattaaagtggacagtaataagtaaatgtccttatta

SEQ ID NO: 162 - DNA, 60 bp, Artificial Sequence
  DNA probe for hepatotoxicity score prediction
accatcctgtaaagctcttgtaccgctggagaaatggcatcactataagctatgagttga

SEQ ID NO: 163 - DNA, 60 bp, Artificial Sequence
  DNA probe for hepatotoxicity score prediction
acggatgacactgaactttacttgtcactatactcttctcatttttcccgaccacttaga

SEQ ID NO: 164 - DNA, 60 bp, Artificial Sequence
  DNA probe for hepatotoxicity score prediction
aagtctgtctactgccctcttcagtctgtaacacttatattggccatgaactgtacctgc

SEQ ID NO: 165 - DNA, 60 bp, Artificial Sequence
  DNA probe for hepatotoxicity score prediction
tctttagcacaaccaggcccctctttgagcctcgtgaagaatttggatgtctgttattta

SEQ ID NO: 166 - DNA, 60 bp, Artificial Sequence
  DNA probe for hepatotoxicity score prediction
gacaggtcaactgataaagtagcactgagatagagggaactgttattaaagggtgttttt

SEQ ID NO: 167 - DNA, 60 bp, Artificial Sequence
  DNA probe for hepatotoxicity score prediction
aattggcgatgaactggacaacaacatggagctgcagaggatgattgctgatgtggatac

SEQ ID NO: 168 - DNA, 60 bp, Artificial Sequence
  DNA probe for hepatotoxicity score prediction
ctgaggcagtcaatttaattgaggttttggaagaaaaaacttgttcatgggctgtttcta

SEQ ID NO: 169 - DNA, 60 bp, Artificial Sequence
  DNA probe for hepatotoxicity score prediction
ccttatccctaaggggacaaccctcatcaccaacctgtcctcagtgctgaaggatgagac

SEQ ID NO: 170 - DNA, 60 bp, Artificial Sequence
  DNA probe for hepatotoxicity score prediction
gtcaaccaacttcatgattcaatcatagaagatgaaaatctgtctgataaacagaagtcc

SEQ ID NO: 171 - DNA, 60 bp, Artificial Sequence
  DNA probe for hepatotoxicity score prediction
agaacttaaaaaatttgaacctaaactgaggtggaaaagacacagttagctaggattgac

SEQ ID NO: 172 - DNA, 60 bp, Artificial Sequence
  DNA probe for hepatotoxicity score prediction
ttaggactatggttttcccagtgatgtatctcaatgagagtgttctcattgacaaagaga

SEQ ID NO: 173 - DNA, 60 bp, Artificial Sequence
  DNA probe for hepatotoxicity score prediction
gggtatggctcctacataatctggaaagagctgggaggtttcacagaggaggctatggtt

SEQ ID NO: 174 - DNA, 60 bp, Artificial Sequence
  DNA probe for hepatotoxicity score prediction
gtggatgaatatgatcctacgatagaggactcctacaggaaacaagtagtaattgatgga

SEQ ID NO: 175 - DNA, 60 bp, Artificial Sequence
  DNA probe for hepatotoxicity score prediction
aaacactgtaggagggcgatatgttttagcacctttgagcatttactttatggagaatat

SEQ ID NO: 176 - DNA, 60 bp, Artificial Sequence
  DNA probe for hepatotoxicity score prediction
catcaggcctgaattttctcagccatgcccatttcatgctctgaggtttgggattgggat

SEQ ID NO: 177 - DNA, 60 bp, Artificial Sequence
  DNA probe for hepatotoxicity score prediction
aggaaggctaattagtaactgtataataggatactaggaactctgtaaaactgtgctcta

SEQ ID NO: 178 - DNA, 60 bp, Artificial Sequence
  DNA probe for hepatotoxicity score prediction
ggcacctttctttattagctacaatgagaccaatagctcacaaagtattgtgttttgaca

SEQ ID NO: 179 - DNA, 60 bp, Artificial Sequence
  DNA probe for hepatotoxicity score prediction
aaagcatgtcctttgcaacctgatcacagagatgatgaaaatgaatagaacttattcatc

SEQ ID NO: 180 - DNA, 60 bp, Artificial Sequence
  DNA probe for hepatotoxicity score prediction
agccgcaaagagaaacagaggagtctggcgacaacagatttaatactgactggctgtttt

SEQ ID NO: 181 - DNA, 60 bp, Artificial Sequence
  DNA probe for hepatotoxicity score prediction
ggctagggatccagatgtagtggaatttattatttgttgagtcctgaacctttgagcctg

SEQ ID NO: 182 - DNA, 60 bp, Artificial Sequence
  DNA probe for hepatotoxicity score prediction
cattgcttggaatttggggtgaaatctgaaaagatttaaggaggttgagggtgtggcact

SEQ ID NO: 183 - DNA, 60 bp, Artificial Sequence
  DNA probe for hepatotoxicity score prediction
gggtaccccatcttcctgatttatccctttgagcctggggtttatacccacagcccttag

SEQ ID NO: 184 - DNA, 60 bp, Artificial Sequence
  DNA probe for hepatotoxicity score prediction
aggcaacgtttgtaatggattaaatccagctttattttaggtgaactgtcctgttggaag

SEQ ID NO: 185 - DNA, 60 bp, Artificial Sequence
  DNA probe for hepatotoxicity score prediction
acctcaatggagatggacgagtggacttccgaagatttgtccggatgatgtctcgctgag

SEQ ID NO: 186 - DNA, 60 bp, Artificial Sequence
  DNA probe for hepatotoxicity score prediction
aggcttggctttcaaaacaaaacacttcccagagagcaatcatcaataaagattgatagc

SEQ ID NO: 187 - DNA, 60 bp, Artificial Sequence
  DNA probe for hepatotoxicity score prediction
acattaaactcgggtcatcctgaactgggacttttcacagaggcgtgatttaaaagaaag

SEQ ID NO: 188 - DNA, 60 bp, Artificial Sequence
  DNA probe for hepatotoxicity score prediction
gcaaaagaagctaatgtgaaatgtccacaaattgtgatagcattttatgaagagagactg

SEQ ID NO: 189 - DNA, 60 bp, Artificial Sequence
  DNA probe for hepatotoxicity score prediction
ggccatgttcttttcctcagtttggatcaggaagaaggaactttaatgactgtgcttcct

SEQ ID NO: 190 - DNA, 60 bp, Artificial Sequence
  DNA probe for hepatotoxicity score prediction
ttgctaatcattcagtaaatccaaactgctatgcaaaagttatgatggttaatggtgacc

SEQ ID NO: 191 - DNA, 60 bp, Artificial Sequence
  DNA probe for hepatotoxicity score prediction
cagccagcacagtggtccctcttgactaccagctcttgacactacctttttcttttaaaa

SEQ ID NO: 192 - DNA, 60 bp, Artificial Sequence
  DNA probe for hepatotoxicity score prediction
tttcagatgacaaatcctgttactgtccttttcattaataaatagatgtacactaacaat

SEQ ID NO: 193 - DNA, 60 bp, Artificial Sequence
  DNA probe for hepatotoxicity score prediction
ctaggttccgacttaatgcaaagggagaaagaaattgcttccttaaaggaaaaaatatct

SEQ ID NO: 194 - DNA, 60 bp, Artificial Sequence
  DNA probe for hepatotoxicity score prediction
tttttaaatatgagtctgaagacacgtcactatattccaggaaattcaaaccgagattgg

SEQ ID NO: 195 - DNA, 60 bp, Artificial Sequence
  DNA probe for hepatotoxicity score prediction
ctaaaggctgcacctttgtgtataaattggaataaattagtacatcctaaatataaaaaa

SEQ ID NO: 196 - DNA, 60 bp, Artificial Sequence
  DNA probe for hepatotoxicity score prediction
cctccctcaggcactaacttatacagctaagattgagctttccaaagtatttaaaataaa

SEQ ID NO: 197 - DNA, 60 bp, Artificial Sequence
  DNA probe for hepatotoxicity score prediction
ctctacacctttcaacagaccacagttttcccagaaaggatccggaatatgtttcccaac

SEQ ID NO: 198 - DNA, 60 bp, Artificial Sequence
  DNA probe for hepatotoxicity score prediction
aaacactacagtgccttataattcactatctgtggacttctaaaggactggatggttttg

SEQ ID NO: 199 - DNA, 60 bp, Artificial Sequence
  DNA probe for hepatotoxicity score prediction
gcacacacagtactagcaggcagtaactggataccttttatttgaacaaacaaacggggt

SEQ ID NO: 200 - DNA, 60 bp, Artificial Sequence
  DNA probe for hepatotoxicity score prediction
ggaagaagattccacatatcatcttgcttgtacatttaaaaagatttggcattcatgagg

SEQ ID NO: 201 - DNA, 60 bp, Artificial Sequence
  DNA probe for hepatotoxicity score prediction
cttctgccttttgtatctcactctcaagtgtacatcacacacagaatgtgaatgtggtca

SEQ ID NO: 202 - DNA, 60 bp, Artificial Sequence
  DNA probe for hepatotoxicity score prediction
aggtaattagtccggaaacttcgctcattcagcaagacaagcagtcaagtctcttacaga

SEQ ID NO: 203 - DNA, 60 bp, Artificial Sequence
  DNA probe for hepatotoxicity score prediction
tcacgtctaagcattgtcgtagactttgtggctctgacatacttgtctgttgagagtcca

SEQ ID NO: 204 - DNA, 60 bp, Artificial Sequence
  DNA probe for hepatotoxicity score prediction
cttgaccgtgctaactgtgtgtacatatatattctacatatatgtatattaaacccgcac

SEQ ID NO: 205 - DNA, 60 bp, Artificial Sequence
  DNA probe for hepatotoxicity score prediction
gttaatgggtgggaatgactgactgtatgttgaggatctattactgactgtatggcgagg

SEQ ID NO: 206 - DNA, 60 bp, Artificial Sequence
  DNA probe for hepatotoxicity score prediction
gtaaatactccccatactagcttttcctacatgagtctacataataaaatggtgaacaag

SEQ ID NO: 207 - DNA, 60 bp, Artificial Sequence
  DNA probe for hepatotoxicity score prediction
atccttatcatccttagcaaaagatgccggtgcaaattcaaccaacagcagagaactggg

SEQ ID NO: 208 - DNA, 60 bp, Artificial Sequence
  DNA probe for hepatotoxicity score prediction
ttcagaaccagcaaagctcagacttggactctgatgttgaagattactaacctcctgctc

SEQ ID NO: 209 - DNA, 60 bp, Artificial Sequence
  DNA probe for hepatotoxicity score prediction
caactggctgtaggagaagttatggccaaaactttttacagaattattttgtaccattag

SEQ ID NO: 210 - DNA, 60 bp, Artificial Sequence
  DNA probe for hepatotoxicity score prediction
tccaaatgtggttgaggctcatagcatccaactggaaaacgacgagatatgagctagaca

SEQ ID NO: 211 - DNA, 60 bp, Artificial Sequence
  DNA probe for hepatotoxicity score prediction
cactcttcaagtcacgtgaaaacaggaagtaaacaggaaataaactaaaaccactccaga

SEQ ID NO: 212 - DNA, 60 bp, Artificial Sequence
  DNA probe for hepatotoxicity score prediction
ccgtgggggataggtttgaaactaaagtctaggcttatgatagctttgtaaataaatccg

SEQ ID NO: 213 - DNA, 60 bp, Artificial Sequence
  DNA probe for hepatotoxicity score prediction
aagatgatgaagaacttgagcccgaagtctgagaagttatcttgtagtgagacctgtgtg

SEQ ID NO: 214 - DNA, 60 bp, Artificial Sequence
  DNA probe for hepatotoxicity score prediction
tccaacctacacctgtacacatttgttttggtccctatcttaataaagctcagaaattcc

SEQ ID NO: 215 - DNA, 60 bp, Artificial Sequence
  DNA probe for hepatotoxicity score prediction
ttccccagaaatcatcgagaccaccttataccagcgttatgagatcaagatgactaagat

SEQ ID NO: 216 - DNA, 60 bp, Artificial Sequence
  DNA probe for hepatotoxicity score prediction
ctgtccctaaaggcagatagaaggcttcttgctgtttagatatttctaggtgaggagggt

SEQ ID NO: 217 - DNA, 60 bp, Artificial Sequence
  DNA probe for hepatotoxicity score prediction
tttaaccaggactctgaaactcaggaatagtggtcatagctgtaaagacaaaaccaaggc

SEQ ID NO: 218 - DNA, 60 bp, Artificial Sequence
  DNA probe for hepatotoxicity score prediction
gaaaacagagacatgaggaaacttgaatatgtgtgtctgagtattcctttgctaagaaat

SEQ ID NO: 219 - DNA, 60 bp, Artificial Sequence
  DNA probe for hepatotoxicity score prediction
tacttcagtctctccatttacacagcttctttaactgagatatggaaagaaataaatggc

SEQ ID NO: 220 - DNA, 60 bp, Artificial Sequence
  DNA probe for hepatotoxicity score prediction
gtcttgagatttgtttactcttgttagcagtcattactacgttaagagtaaaccaaagca

SEQ ID NO: 221 - DNA, 60 bp, Artificial Sequence
  DNA probe for hepatotoxicity score prediction
ttagggaggcacccaaaggacactacatacgaccaaggattattaaacagaacacttgct

SEQ ID NO: 222 - DNA, 60 bp, Artificial Sequence
  DNA probe for hepatotoxicity score prediction
aaatagacattcgttggaaatatcatctgccctaaatatgttcaacatttcacctcacgg

SEQ ID NO: 223 - DNA, 60 bp, Artificial Sequence
  DNA probe for hepatotoxicity score prediction
aaagcacttgaatgatgaatcgacttccaaacagattcgagcgatgcttcagtagaaccc

SEQ ID NO: 224 - DNA, 60 bp, Artificial Sequence
  DNA probe for hepatotoxicity score prediction
gaagtcaaaaggcaaatctgtcttgtcatgttgtaaaatgctactgttctttgttgaaga

SEQ ID NO: 225 - DNA, 60 bp, Artificial Sequence
  DNA probe for hepatotoxicity score prediction
ggtaaaagctatggtttaaaggccattggacttccaacatttacaagttcattagaatag

SEQ ID NO: 226 - DNA, 60 bp, Artificial Sequence
  DNA probe for hepatotoxicity score prediction
gtgcggtctaagaactgacagtagcaacctctgatatcctgaatctgcattttcccagaa

SEQ ID NO: 227 - DNA, 60 bp, Artificial Sequence
  DNA probe for hepatotoxicity score prediction
gctctttgtagagagtttggaaactgtaggagattgttgagaagaaaaataaaaatcagc

SEQ ID NO: 228 - DNA, 60 bp, Artificial Sequence
  DNA probe for hepatotoxicity score prediction
aggatgtcatctgtaccagccgcgactgtcccatcttctacatgcgcaagaaggtgcgca

SEQ ID NO: 229 - DNA, 60 bp, Artificial Sequence
  DNA probe for hepatotoxicity score prediction
atgaggtaagacagcccttgttttcaattttataggtagaattcagtcataaagagctgg

SEQ ID NO: 230 - DNA, 60 bp, Artificial Sequence
  DNA probe for hepatotoxicity score prediction
gagttggacgtaccgtctgtgacatgagacacttcctcatatgtgtcgtgaataaaccat

SEQ ID NO: 231 - DNA, 60 bp, Artificial Sequence
  DNA probe for hepatotoxicity score prediction
ttataaaaacaaagccctattcacattaggtgacttgctatatagcacgagcttccttag

SEQ ID NO: 232 - DNA, 60 bp, Artificial Sequence
  DNA probe for hepatotoxicity score prediction
taaaaagggtttgtagctttttcaggggctacaacgtacagttagatttaaagcttttga

SEQ ID NO: 233 - DNA, 60 bp, Artificial Sequence
  DNA probe for hepatotoxicity score prediction
tgtgccaacccagagaagaagtgggttcaagaatacatcaactatttggagatgagctag

SEQ ID NO: 234 - DNA, 60 bp, Artificial Sequence
  DNA probe for hepatotoxicity score prediction
tgccgaagcttactaatgctaagggctggcccagattattaagctaataaaaaatatcgt

SEQ ID NO: 235 - DNA, 60 bp, Artificial Sequence
  DNA probe for hepatotoxicity score prediction
tctggatacaggaatgcatgacattgcaaaacaattcttaaagcaaagtttcaattgctc

SEQ ID NO: 236 - DNA, 60 bp, Artificial Sequence
  DNA probe for hepatotoxicity score prediction
ccatcaagactccccccagtggtttacttgcaaaaataaaagctgcagaagctcaaaaaa

SEQ ID NO: 237 - DNA, 60 bp, Artificial Sequence
  DNA probe for hepatotoxicity score prediction
gtcaccaatacaccaatatgtttcaattcttcatccccgcctactggtcctgtttttaga

SEQ ID NO: 238 - DNA, 60 bp, Artificial Sequence
  DNA probe for hepatotoxicity score prediction
tcttataagaagttccgcttactaccatgtctccaccttgctggaaaggccaagcagaaa

(19) United States
(12) Patent Application Publication
(10) Pub. No.: US 2005/0143628 A1
(43) Pub. Date: Jun. 30, 2005

(54) Methods for characterizing tissue or organ condition or status

(76) Inventors: Xudong Dai, Kirkland, WA (US); Yudong He, Kirkland, WA (US); Hongyue Dai, Bothell, WA (US); Roger G. Ulrich, Sammamish, WA (US); Jeffrey F. Waring, Franklin, WI (US)

Correspondence Address: JONES DAY, 222 EAST 41ST ST, NEW YORK, NY, 10017, US

(21) Appl. No.: 10/872,157

(22) Filed: Jun. 17, 2004

Related U.S. Application Data
(60) Provisional application No. 60/479,817, filed on Jun. 18, 2003.

(51) Int. Cl. G06F 19/00; G01N 33/48; G01N 33/50; A61B 5/00

(52) U.S. Cl. 600/300; 702/19

(57) ABSTRACT

The invention provides methods for characterizing the condition or status of a tissue or organ in a multicellular organism, e.g., an animal, by combining a plurality of clinical measures are combined into a composite clinical score (CCS) and using such a CCS to represent the condition or status of the tissue or organ. The invention provides methods for predicting the condition or status of a tissue or organ in a multicellular organism, e.g., an animal, based on measurements of a set of cellular constituent markers, e.g., measured expression levels of a set of marker genes. The invention also provides methods for selecting the set of marker genes whose expression levels can be used in determining the CCS.

This application claims the benefit under 35 U.S.C. § 119(e) of U.S. Provisional Patent Application No. 60/479,817, filed on Jun. 18, 2003, which is incorporated by reference herein in its entirety.

### 1. FIELD OF THE INVENTION

The present invention relates to methods of characterizing tissue or organ condition or status. The invention also relates to methods for prediction of drug toxicity based on transcriptional profiles.

### 2. BACKGROUND OF THE INVENTION

DNA array technologies have made it possible to monitor the expression level of a large number of genetic transcripts at any one time (see, e.g., Schena et al., 1995, Science 270: 467-470; Lockhart et al., 1996, Nature Biotechnology 14: 1675-1680; Blanchard et al., 1996, Nature Biotechnology 14: 1649; Ashby et al., U.S. Pat. No. 5,569,588, issued Oct. 29, 1996). Of the two main formats of DNA arrays, spotted cDNA arrays are prepared by depositing PCR products of cDNA fragments with sizes ranging from about 0.6 to 2.4 kb, from full length cDNAs, ESTs, etc., onto a suitable surface (see, e.g., DeRisi et al., 1996, Nature Genetics 14: 457-460; Shalon et al., 1996, Genome Res. 6: 689-645; Schena et al., 1995, Proc. Natl. Acad. Sci. U.S.A. 93: 10539-11286; and Duggan et al., Nature Genetics Supplement 21: 10-14). Alternatively, high-density oligonucleotide arrays containing thousands of oligonucleotides complementary to defined sequences, at defined locations on a surface are synthesized in situ on the surface by, for example, photolithographic techniques (see, e.g., Fodor et al., 1991, Science 251: 767-773; Pease et al., 1994, Proc. Natl. Acad. Sci. U.S.A. 91: 5022-5026; Lockhart et al., 1996, Nature Biotechnology 14: 1675; McGall et al., 1996, Proc. Natl. Acad. Sci. U.S.A. 93: 13555-13560; U.S. Pat. Nos. 5,578,832; 5,556,752; 5,510,270; and 6,040,138). Methods for generating arrays using inkjet technology for in situ oligonucleotide synthesis are also known in the art (see, e.g., Blanchard, International Patent Publication WO 98/41531, published Sep. 24, 1998; Blanchard et al., 1996, Biosensors and Bioelectronics 11: 687-690; Blanchard, 1998, in Synthetic DNA Arrays in Genetic Engineering, Vol. 20, J. K. Setlow, Ed., Plenum Press, New York at pages 111-123). Efforts to further increase the information capacity of DNA arrays range from further reducing feature size on DNA arrays so as to further increase the number of probes in a given surface area to sensitivity- and specificity-based probe design and selection aimed at reducing the number of redundant probes needed for the detection of each target nucleic acid thereby increasing the number of target nucleic acids monitored without increasing probe density (see, e.g., Friend et al., International Publication No. WO 01/05935, published Jan. 25, 2001).

By simultaneously monitoring tens of thousands of genes, DNA array technologies have allowed, inter alia, genome-wide analysis of mRNA expression in a cell or a cell type or any biological sample. Aided by sophisticated data management and analysis methodologies, the transcriptional state of a cell or cell type as well as changes of the transcriptional state in response to external perturbations, including but not limited to drug perturbations, can be characterized on the mRNA level (see, e.g., Stoughton et al., International Publication No. WO 00/39336, published Jul. 6, 2000; Friend et al., International Publication No. WO 00/24936, published May 4, 2000; and Shoemaker et al., International Publication No. WO 02/16650, published Feb. 28, 2002). Applications of such technologies include, for example, identification of genes which are up regulated or down regulated in various physiological states, particularly diseased states. Additional exemplary uses for DNA arrays include the analyses of members of signaling pathways, and the identification of targets for various drugs. See, e.g., Friend and Hartwell, International Publication No. WO 98/38329 (published Sep. 3, 1998); Stoughton, International Publication No. WO 99/66067 (published Dec. 23, 1999); Stoughton and Friend, International Publication No. WO 99/58708 (published Nov. 18, 1999); Friend and Stoughton, International Publication No. WO 99/59037 (published Nov. 18, 1999); Friend et al., U.S. Pat. No. 6,218,122.

Drug induced adverse effect is among the top five causes of disease related death. Lethal hepatotoxicity is the primary reason why a majority of new drugs are being withdrawn from the market. To develop safer drugs, it is critical to improve the accuracy of compound hepatotoxicity estimation. The development of global expression profiling based on microarray and other platforms may provide a potential solution to this problem (Waring et al., 2000, Annu Rev Pharmacol Toxicol 40: 335-352.; He et al., 2001, Nat Med 7: 658-659; Friend, 2002, Sci Am 286: 44-49, 53; Hamadeh et al., 2002, Curr Issues Mol Biol 4: 45-56; Ulrich et al., 2002, Nat Rev Drug Discov 1: 84-88; Waring, 2002, Curr Opin Mol Ther 4: 229-235). An increasing number of publications have reported the utilization of microarrays to identify discrete gene sets associated with a specific toxic response (Hamadeh et al., 2002, Toxicol Sci 67: 219-231; Hamadeh et al., 2002, Toxicol Pathol 30: 470-482) or to conduct surveys on genes affected by compounds with known mechanisms (Bouton et al., 2000, Neurotoxicology 21: 1045-1055; Brambila et al., 2002, J Toxicol Environ Health A 65: 1273-1288; Bulera et al., 2001, Hepatology 33: 1239-1258; Burczynski et al., 2000, Toxicol Sci 58: 399-415; Chen et al, 2001, Mol Carcinog 30: 79-87).

To date, two major types of analytical methods have been employed in the majority of reported expression profiling studies about compound toxicity (Marton et al., 1998, Nat Med 4: 1293-1301; Bartosiewicz et al., 2000, Arch Biochem Biophys 376: 66-73; Bouton et al., 2000, Neurotoxicology 21: 1045-1055; Burczynski et al., 2000, Toxicol Sci 58: 399-415; Cunningham et al., 2000, Ann N Y Acad Sci 919: 52-67; Hughes, Marton et al. 2000, Cell 102: 109-126; Nadadur et al., 2000, Inhal Toxicol 12: 1239-1254; Baker, Carfagna et al. 2001, Chem Res Toxicol 14: 1218-1231; Bartosiewicz et al., 2001, Environ Health Perspect 109(1): 71-74; Bouton et al., 2001, Toxicol Appl Pharmacol 176: 34-53; Bulera et al., 2001, Hepatology 33: 1239-1258; Huang et al., 2001, Toxicol Sci 63: 196-207; Lu et al., 2001, Toxicol Sci 59(1): 185-92; Reilly et al., 2001, Biochem Biophys Res Commun 282: 321-328; Waring et al., 2001, Toxicol Lett 120: 359-368; Waring et al., 2001, Toxicol Appl Pharmacol 175: 28-42; Brambila et al., 2002, J Toxicol Environ Health A 65: 1273-1288; Donald et al., 2002, Cancer Res 62: 4256-4262; Hamadeh et al., 2002, Toxicol Sci 67: 219-231; Hamadeh et al., 2002, Toxicol Pathol 30: 470-482; Li et al., 2002, J Biol Chem 277: 388-394; Yamada et al, 2002, Ind Health 40: 159-166; Yih et al., 2002, Carcinogenesis 23: 867-876). The first one is a type of clustering approach, often referred to as an “unsupervised clustering approach,” which includes hierarchical, k-mean clustering, a self-organizing map, etc. This approach allows compounds to be clustered based on their gene expression similarities across a set of genes differentially regulated by many treatments. Toxicity of compounds within the same cluster is determined ad hoc using “guilt by association.” The second type of analytical method is often referred to by biologists as a “supervised clustering approach,” or “classification approach.” In particular, a set of genes commonly regulated by a compound or a set of compounds associated with a type of well studied toxicity, such as DNA damage, apoptosis and so on, is first identified as the reference gene set. A template is then established from the reference gene set. The toxicity of an unknown compound is “classified” based on the similarity or distance between the expression profile of the established template and the unknown compound.

Limitations exist in the above analytical approaches for ab initio prediction of compound toxicity. First, the toxicity prediction from these approaches is not quantitative. It is often essential to obtain a quantitative toxicity measurement from the expression profile in order to compare the relative toxicities among different compounds at a certain dose. Second, the accuracy and generality of the toxicity prediction from the above approaches have not yet been examined. Without testing the accuracy and generality, the reference gene set for a certain type of toxicity may reflect nothing but a specific pharmacological response. Third, none of the above approaches associates the expression profile from each individual animal with its actual toxic response. It has been observed repeatedly and accepted widely that a huge variance could exist in the toxic response because of genetic, environmental and physiological differences among individual animals.

Recently, more sophisticated and powerful machine learning algorithms have been applied to transcriptional profiling analysis. For example, a modified “Fisher classification” approach has been applied to distinguish patients with good prognosis from those who do not, based on their expression profiles (van 't Veer et al., 2002, Nature 415: 530-6). A similar study has been reported using an artificial neural network (Khan et al., 2001, Nat Med 7: 673-9). However, in most of these studies, the variables to be predicted are well defined, such as survival time or some clinical measurements. Due to the complexity of liver injury, none of those measurements accepted in clinical practice or academic study can comprehensively represent the degree of liver damage induced by hepatotoxicants.

Thus, development of a systematic approach for ab initio prediction of compound hepatotoxicity based on a compendium of expression profiles was a challenging task in the prior art. Such an approach is provided by the present invention.

Discussion or citation of a reference herein shall not be construed as an admission that such reference is prior art to the present invention.

### 3. SUMMARY OF THE INVENTION

The invention provides methods for characterizing the condition or status of a tissue or organ in a multicellular organism, e.g., an animal, by combining a plurality of clinical measures are combined into a composite clinical score (CCS) and using such a CCS to represent the condition or status of the tissue or organ. The invention also provides methods for predicting the condition or status of a tissue or organ in a multicellular organism, e.g., an animal, based on measurements of a set of cellular constituent markers, e.g., measured expression levels of a set of marker genes. The methods of the invention involves using a machine learning algorithm to build a model for determining a suitable composite clinical score of the tissue or organ based on measurements of a set of cellular constituent markers. The invention also provides methods for reduction of variable dimension of response profiles, e.g., by transforming a profile into a feature space of reduced dimension using, e.g., a wavelet transformation.

In one aspect of the invention, the invention provides a method for characterizing the condition of a tissue or organ in an animal, comprising determining a composite clinical score of the tissue or organ, where the composite clinical score is determined based on a plurality of k clinical measures of the tissue or organ of the animal. In preferred embodiments, each of the k clinical measures is a converted clinical measure represented as deviations from the respective normal value. In one embodiment, each of the converted clinical measures is calculated according to the equation

$${D}_{i} = \frac{{x}_{i} - {μ}_{i , 0}}{{σ}_{i , 0}}$$

wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k. In another embodiment, each of the plurality of k clinical measures is sigmoidal transformed according to the equation

$$\begin{matrix}{D}_{i}^{′} = \frac{1 - {ⅇ}^{- {α}_{i}}}{1 + {ⅇ}^{- {α}_{i}}} \\ where \\ {α}_{i} = \frac{{D}_{i} - {\overline{D}}_{i}}{{c}_{i} \cdot Std ( {\overline{D}}_{i} )}\end{matrix}$$

wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k. In one embodiment, the composite clinical score is calculated according to the equation

$$CCS = {\sum }_{i = 1}^{k} {β}_{i} \cdot {D}_{i}^{′}$$

where CCS designates the composite clinical score, and where βᵢ is a coefficient of the ith converted clinical measure, and i=1, 2, . . . , k.

The condition of the tissue or organ can be a disease condition, such as an inflammation or damage. In another embodiment, the tissue or organ is further classified according to a predetermined threshold of the composite clinical score, where the tissue or organ is classified into one or the other category depending on if the composite clinical score is greater or smaller than the predetermined threshold.

In specific embodiments, the organ is liver and the plurality of k clinical measures are selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time. In a preferred embodiment, the plurality of k clinical measures consist of the serum level of alanine aminotransferase (ALT), the serum level of aspartate aminotransferase (AST), the serum level of alkaline phosphatase (ALP), the serum level of total bilirubin (Tbil), and the serum level of cholesterol (Chol). In a preferred embodiment, the serum level of alanine aminotransferase (ALT) is sigmoidal transformed with c of 3, and the serum level of alkaline phosphatase (ALP), the serum level of total bilirubin (Tbil), and the serum level of cholesterol (Chol) are each sigmodal transformed with c of 1. In another preferred embodiment, the composite clinical score is a hepatotoxicity score HS calculated according to the equation

$$\begin{matrix}HS = {D}_{Tbil}^{′} ( if Tbil is abnormal ) + \\ 0.5 {D}_{ALP}^{′} + 3 {D}_{ALT}^{′} + 1.5 {D}_{AST}^{′} + \\ 0.3 {D}_{Chol}^{′} ( if both Chol and at least one \\ other clinical measure are abnormal )\end{matrix}$$

In one embodiment, the invention provides a computer system comprising a processor, and a memory coupled to the processor and encoding one or more programs, which cause the processor to carry out the method of the invention. In another embodiment, the invention provides a computer program product for use in conjunction with a computer having a processor and a memory connected to the processor, the computer program product comprising a computer readable storage medium having a computer program mechanism encoded thereon, wherein the computer program mechanism may be loaded into the memory of the computer and cause the computer to carry out the method of the invention.

In another aspect, the invention provides a method for characterizing the condition of a tissue or organ in an animal, comprising determining a composite clinical score of the tissue or organ based on a cellular constituent profile of the tissue or organ, where the cellular constituent profile comprises measurements of a plurality of cellular constituents in cells of the tissue or organ. In one embodiment, the composite clinical score of the tissue or organ is determined by a model estimator according to equation
CCS=f(z₁, z₂, . . . zₙ)
where {z₁, x₂, . . . , Zₙ} are data characterizing the cellular constituent profile. In preferred embodiments, the {z₁, z₂, . . . , zₙ} are data in a feature space. In one embodiment, the {z₁, z₂, . . . , zₙ} are obtained by transforming the cellular constituent profile using a wavelet transformation of a suitable level. In one embodiment, the wavelet transformation is a transformation using Daubechies wavelet. In another preferred embodiment, the model estimator is a neural network model.

In one embodiment, the invention provides a computer system comprising a processor, and a memory coupled to the processor and encoding one or more programs, which cause the processor to carry out the method of the invention. In another embodiment, the invention provides a computer program product for use in conjunction with a computer having a processor and a memory connected to the processor, the computer program product comprising a computer readable storage medium having a computer program mechanism encoded thereon, wherein the computer program mechanism may be loaded into the memory of the computer and cause the computer to carry out the method of the invention.

The invention also provides a computer program encoding a model estimator for characterizing a condition of a tissue or organ in an animal, the computer program accepting data characterizing a cellular constituent profile of the tissue or organ, where the cellular constituent profile comprises measurements of a plurality of cellular constituent in cells of the tissue or organ, and outputting a composite clinical score of the tissue or organ, where the composite clinical score indicates the condition of the tissue or organ of the animal. Preferably, the data characterizing the cellular constituent profile are data in a feature space. In one embodiment, the data in the feature space are obtained by transforming the cellular constituent profile using a wavelet transformation of a suitable level. In one embodiment, the wavelet transformation is a transformation using Daubechies wavelets of a suitable level. In a preferred embodiment, the model estimator is a neural network model. In one embodiment, the computer program is for characterizing a condition which results from a perturbation to the tissue or organ. The perturbation can be a drug perturbation and where the condition of the tissue or organ results from the toxicity of the drug.

In still another aspect, the invention provides a method for evaluating the toxicity of a drug to a tissue or organ in an animal, comprising determining a composite clinical score of the tissue or organ based on a cellular constituent profile of the tissue or organ, wherein the cellular constituent profile comprises measurements of a plurality of cellular constituent in cells of the tissue or organ after administration of the drug to the animal. In one embodiment, the composite clinical score of the tissue or organ is determined by a model estimator according to equation
CCS=f(z₁, z₂, . . . zₙ)
where {z₁, z₂, . . . , zₙ} are data characterizing the cellular constituent profile. In preferred embodiments, the {z₁, z₂, . . . , zₙ} are data in a feature space. In one embodiment, the {z₁, z₂, . . . zₙ} are obtained by transforming the cellular constituent profile using a wavelet transformation of a suitable level. In one embodiment, the wavelet transformation is a transformation using Daubechies wavelet. In another preferred embodiment, the model estimator is a neural network model.

Preferably, the composite clinical score is a combination of a plurality of k clinical measures of the tissue or organ of the animal. In preferred embodiments, each of the k clinical measures is a converted clinical measure represented as deviations from the respective normal value. In one embodiment, each of the converted clinical measures is calculated according to the equation

$${D}_{i} = \frac{{x}_{i} - {μ}_{i , 0}}{{σ}_{i , 0}}$$

wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k. In another embodiment, each of the plurality of k clinical measures is sigmoidal transformed according to the equation

$$\begin{matrix}{D}_{i}^{′} = \frac{1 - {ⅇ}^{- {α}_{i}}}{1 + {ⅇ}^{- {α}_{i}}} \\ where \\ {α}_{i} = \frac{{D}_{i} - {\overline{D}}_{i}}{{c}_{i} \cdot Std ( {\overline{D}}_{i} )}\end{matrix}$$

wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k. In one embodiment, the composite clinical score is calculated according to the equation

$$CCS = {\sum }_{i = 1}^{k} {β}_{i} \cdot {D}_{i}^{′}$$

where CCS designates the composite clinical score, and where βᵢ is a coefficient of the ith converted clinical measure, and i=1, 2, . . . , k.

In specific embodiments, the organ is liver and the plurality of k clinical measures are selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time. In a preferred embodiment, the plurality of k clinical measures consist of the serum level of alanine aminotransferase (ALT), the serum level of aspartate aminotransferase (AST), the serum level of alkaline phosphatase (ALP), the serum level of total bilirubin (Tbil), and the serum level of cholesterol (Chol). In a preferred embodiment, the serum level of alanine aminotransferase (ALT) is sigmoidal transformed with c of 3, and the serum level of alkaline phosphatase (ALP), the serum level of total bilirubin (Tbil), and the serum level of cholesterol (Chol) are each siginodal transformed with c of 1. In another preferred embodiment, the composite clinical score is a hepatotoxicity score HS calculated according to the equation

$$\begin{matrix}HS = {D}_{Tbil}^{′} ( if Tbil is abnormal ) + \\ 0.5 {D}_{ALP}^{′} + 3 {D}_{ALT}^{′} + 1.5 {D}_{AST}^{′} + \\ 0.3 {D}_{Chol}^{′} ( if both Chol and at least one \\ other clinical measure are abnormal )\end{matrix}$$

In a preferred embodiment, the plurality of cellular constituents comprises gene products corresponding to genes or ESTs listed in Table II. In one embodiment, the method further comprises measuring the gene products.

In another embodiment, the drug is further classified according to a predetermined threshold of the composite clinical score, where the drug is classified as causing liver damage if the composite clinical score is greater than the predetermined threshold.

In one embodiment, the invention provides a computer system comprising a processor, and a memory coupled to the processor and encoding one or more programs, which cause the processor to carry out the method of the invention. In another embodiment, the invention provides a computer program product for use in conjunction with a computer having a processor and a memory connected to the processor, the computer program product comprising a computer readable storage medium having a computer program mechanism encoded thereon, wherein the computer program mechanism may be loaded into the memory of the computer and cause the computer to carry out the method of the invention.

In still another aspect, the invention provides a method for evaluating the efficacy of a drug in treating a disease or disorder in a tissue or organ in an animal, comprising (a) determining a composite clinical score of the tissue or organ based on a first cellular constituent profile of the tissue or organ, wherein the first cellular constituent profile comprises measurements of a plurality of cellular constituents in cells of the tissue or organ after administration of the drug to the animal; and (b) comparing the composite clinical score determined in step (a) to (b1) standard values of the composite clinical score indicating condition of the tissue or organ; or (b2) a composite clinical score determined based on a second cellular constituent profile of the tissue or organ, wherein the second cellular constituent profile comprises measurements of the plurality of cellular constituents in cells of the tissue or organ before administration of the drug to the animal; thereby evaluating the efficacy of the drug in treating the disease. It will be understood by one skilled person in the art that disorder include injury or damage to the tissue or organ.

In one embodiment, the composite clinical score of the tissue or organ is determined by a model estimator according to equation
CCS=f(z₁, z₂, . . . , zₙ)
where {z₁, z₂, . . . , zₙ} are data characterizing the cellular constituent profile. In preferred embodiments, the {z₁, z₂, . . . , zₙ} are data in a feature space. In one embodiment, the {z₁, z₂, . . . , zₙ} are obtained by transforming the cellular constituent profile using a wavelet transformation of a suitable level. In one embodiment, the wavelet transformation is a transformation using Daubechies wavelet. In another preferred embodiment, the model estimator is a neural network model.

Preferably, the composite clinical score is a combination of a plurality of k clinical measures of the tissue or organ of the animal. In preferred embodiments, each of the k clinical measures is a converted clinical measure represented as deviations from the respective normal value. In one embodiment, each of the converted clinical measures is calculated according to the equation

$${D}_{i} = \frac{{x}_{i} - {μ}_{i , 0}}{{σ}_{i , 0}}$$

wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k. In another embodiment, each of the plurality of k clinical measures is sigmoidal transformed according to the equation

$$\begin{matrix}{D}_{i}^{′} = \frac{1 - {ⅇ}^{- {α}_{i}}}{1 + {ⅇ}^{- {α}_{i}}} \\ where \\ {α}_{i} = \frac{{D}_{i} - {\overline{D}}_{i}}{{c}_{i} \cdot Std ( {\overline{D}}_{i} )}\end{matrix}$$

wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k. In one embodiment, the composite clinical score is calculated according to the equation

$$CCS = {\sum }_{i = 1}^{k} {β}_{i} \cdot {D}_{i}^{′}$$

where CCS designates the composite clinical score, and where βᵢ is a coefficient of the ith converted clinical measure, and i=1, 2, . . . , k.

In specific embodiments, the organ is liver and the plurality of k clinical measures are selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time. In a preferred embodiment, the plurality of k clinical measures consist of the serum level of alanine aminotransferase (ALT), the serum level of aspartate aminotransferase (AST), the serum level of alkaline phosphatase (ALP), the serum level of total bilirubin (Tbil), and the serum level of cholesterol (Chol). In a preferred embodiment, the serum level of alanine aminotransferase (ALT) is sigmoidal transformed with c of 3, and the serum level of alkaline phosphatase (ALP), the serum level of total bilirubin (Tbil), and the serum level of cholesterol (Chol) are each sigmodal transformed with c of 1. In another preferred embodiment, the composite clinical score is a hepatotoxicity score HS calculated according to the equation

$$HS = {D}_{Tbil}^{′} ( if Tbil is abnormal ) + 0.5 {D}_{ALP}^{′} + \\ 3 {D}_{ALT}^{′} + 1.5 {D}_{AST}^{′} + 0.3 {D}_{Chol}^{′} ( if both \\ Chol and at least on e other \\ clinical measure are abnormal )$$

In a preferred embodiment, the plurality of cellular constituents comprises gene products corresponding to genes or ESTs listed in Table II. In one embodiment, the method further comprises measuring the gene products.

In one embodiment, the invention provides a computer system comprising a processor, and a memory coupled to the processor and encoding one or more programs, which cause the processor to carry out the method of the invention. In another embodiment, the invention provides a computer program product for use in conjunction with a computer having a processor and a memory connected to the processor, the computer program product comprising a computer readable storage medium having a computer program mechanism encoded thereon, wherein the computer program mechanism may be loaded into the memory of the computer and cause the computer to carry out the method of the invention.

In still another aspect, the invention provides a method for determining a model estimator for characterizing a condition of a tissue or organ in an animal, comprising using a plurality of cellular constituent profiles each comprising measurements of a plurality of cellular constituents to train a model estimator, the model estimator outputting a composite clinical score using the measurements of the plurality of cellular constituents in a cellular constituent profile, wherein each of the profiles is obtained from the tissue or organ under a different given condition, and wherein each of the profile has an associated composite clinical score, the composite clinical score being generated using a plurality of clinical measures of the tissue or organ of the animal.

In one embodiment, the composite clinical score of the tissue or organ is determined by a model estimator according to equation
CCS=f(z₁, z₂, . . . zₙ)
where {z₁, z₂, . . . , zₙ} are data characterizing the cellular constituent profile.

In preferred embodiments, the {z₁, z₂, . . . , zₙ} are data in a feature space. In one embodiment, the {z₁, z₂, . . . , zₙ} are obtained by transforming the cellular constituent profile using a wavelet transformation of a suitable level. In one embodiment, the wavelet transformation is a transformation using Daubechies wavelet. In another preferred embodiment, the model estimator is a neural network model.

Preferably, the composite clinical score is determined based on a plurality of k clinical measures of the tissue or organ of the animal. In preferred embodiments, each of the k clinical measures is a converted clinical measure represented as deviations from the respective normal value. In one embodiment, each of the converted clinical measures is calculated according to the equation

$${D}_{i} = \frac{{x}_{i} - {μ}_{i , 0}}{{σ}_{i , 0}}$$

wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k. In another embodiment, each of the plurality of k clinical measures is sigmoidal transformed according to the equation

$${D}_{i}^{′} = \frac{1 - {ⅇ}^{- {α}_{i}}}{1 + {ⅇ}^{- {α}_{i}}}$$

$$where$$

$${α}_{i} = \frac{{D}_{i} - {\overline{D}}_{i}}{{c}_{i} \cdot Std ( {\overline{D}}_{i} )}$$

wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k. In one embodiment, the composite clinical score is calculated according to the equation

$$CCS = {\sum }_{i = 1}^{k} {β}_{i} \cdot {D}_{i}^{′}$$

where CCS designates the composite clinical score, and where βᵢ is a coefficient of the ith converted clinical measure, and i=1, 2, . . . , k.

In specific embodiments, the organ is liver and the plurality of k clinical measures are selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time. In a preferred embodiment, the plurality of k clinical measures consist of the serum level of alanine aminotransferase (ALT), the serum level of aspartate aminotransferase (AST), the serum level of alkaline phosphatase (ALP), the serum level of total bilirubin (Tbil), and the serum level of cholesterol (Chol). In a preferred embodiment, the serum level of alanine aminotransferase (ALT) is sigmoidal transformed with c of 3, and the serum level of alkaline phosphatase (ALP), the serum level of total bilirubin (Tbil), and the serum level of cholesterol (Chol) are each sigmodal transformed with c of 1. In another preferred embodiment, the composite clinical score is a hepatotoxicity score HS calculated according to the equation

$$HS = {D}_{Tbil}^{′} ( if Tbil is abnormal ) + 0.5 {D}_{ALP}^{′} + \\ 3 {D}_{ALT}^{′} + 1.5 {D}_{AST}^{′} + 0.3 {D}_{Chol}^{′} ( if both \\ Chol and at least on e other \\ clinical measure are abnormal )$$

In a preferred embodiment, the plurality of cellular constituents comprises gene products corresponding to genes or ESTs listed in Table II. In one embodiment, the method further comprises measuring the gene products.

In one embodiment, the condition results from a perturbation to the animal, and the model estimator is used for characterizing an effect of the perturbation on the tissue or organ. In one embodiment, the perturbation is administration of drug to the animal and the effect is a toxicity of the drug.

In preferred embodiments, the plurality of cellular constituent profiles consists of at least 100, at least 1,000, or at least 10,000 profiles.

In one embodiment, the invention provides a computer system comprising a processor, and a memory coupled to the processor and encoding one or more programs, which cause the processor to carry out the method of the invention. In another embodiment, the invention provides a computer program product for use in conjunction with a computer having a processor and a memory connected to the processor, the computer program product comprising a computer readable storage medium having a computer program mechanism encoded thereon, wherein the computer program mechanism may be loaded into the memory of the computer and cause the computer to carry out the method of the invention.

In other embodiments, the method further comprises before the step of using, a step of selecting the plurality of cellular constituent profiles. In still other embodiments, the method further comprises measuring the plurality of profiles of cellular constituents.

In another embodiment, the condition results from a perturbation to the animal, and the model estimator is used for characterizing an effect of the perturbation on the tissue or organ.

In still another aspect, the invention provides a method of determining hepatotoxicity of a compound at a given dosage in an animal, comprising (a) contacting hepatocytic cells of the animal with the compound at the dosage; (b) measuring a cellular constituent profile, wherein the cellular constituent profile comprises measurements of a plurality of cellular constituent in the hepatocytic cells; (c) determining a composite clinical score of the tissue or organ based on the cellular constituent profile; and (d) determining said compound as having hepatotoxicity if said composite clinical score is above a threshold value.

In one embodiment, the composite clinical score of the hepatocytic cells is determined by a model estimator according to equation
CCS=f(z₁, z₂, . . . zₙ)
where {z₁, z₂, . . . , zₙ} are data characterizing the cellular constituent profile. In preferred embodiments, the {z₁, z₂, . . . , zₙ} are data in a feature space. In one embodiment, the {z₁, z₂, . . . , zₙ} are obtained by transforming the cellular constituent profile using a wavelet transformation of a suitable level. In one embodiment, the wavelet transformation is a transformation using Daubechies wavelet. In another preferred embodiment, the model estimator is a neural network model.

Preferably, the composite clinical score is a combination of a plurality of k clinical measures of the hepatocytic cells of the animal. In preferred embodiments, each of the k clinical measures is a converted clinical measure represented as deviations from the respective normal value. In one embodiment, each of the converted clinical measures is calculated according to the equation

$${D}_{i} = \frac{{x}_{i} - {μ}_{i , 0}}{{σ}_{i , 0}}$$

wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k. In another embodiment, each of the plurality of k clinical measures is sigmoidal transformed according to the equation

$${D}_{i}^{′} = \frac{1 - {ⅇ}^{- {α}_{i}}}{1 + {ⅇ}^{- {α}_{i}}}$$

$$where$$

$${α}_{i} = \frac{{D}_{i} - {\overline{D}}_{i}}{{c}_{i} \cdot Std ( {\overline{D}}_{i} )}$$

wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k. In one embodiment, the composite clinical score is calculated according to the equation

$$CCS = {\sum }_{i = 1}^{k} {β}_{i} \cdot {D}_{i}^{′}$$

where CCS designates the composite clinical score, and where βᵢ is a coefficient of the ith converted clinical measure, and i=1, 2, . . . , k.

In specific embodiments, the plurality of k clinical measures are selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time. In a preferred embodiment, the plurality of k clinical measures consist of the serum level of alanine aminotransferase (ALT), the serum level of aspartate aminotransferase (AST), the serum level of alkaline phosphatase (ALP), the serum level of total bilirubin (Tbil), and the serum level of cholesterol (Chol). In a preferred embodiment, the serum level of alanine aminotransferase (ALT) is sigmoidal transformed with c of 3, and the serum level of alkaline phosphatase (ALP), the serum level of total bilirubin (Tbil), and the serum level of cholesterol (Chol) are each sigmodal transformed with c of 1. In another preferred embodiment, the composite clinical score is a hepatotoxicity score HS calculated according to the equation

$$HS = {D}_{Tbil}^{′} ( if Tbil is abnormal ) + 0.5 {D}_{ALP}^{′} + \\ 3 {D}_{ALT}^{′} + 1.5 {D}_{AST}^{′} + 0.3 {D}_{Chol}^{′} ( if both \\ Chol and at least on e other \\ clinical measure are abnormal )$$

In a preferred embodiment, the plurality of cellular constituents comprises gene products corresponding to genes or ESTs listed in Table II. In one embodiment, the method further comprises measuring the gene products.

In another embodiment, the drug is further classified according to a predetermined threshold of the composite clinical score, where the drug is classified as causing liver damage if the composite clinical score is greater than the predetermined threshold.

### 4. BRIEF DESCRIPTION OF FIGURES

FIG. 1A. 2536 differentiated genes were selected based on the criteria of p value <0.01 and |log ratio|>0.05 in at least 3 profiles. Two dimensional clustering of transcript levels of these genes revealed grouping of treatments with the same compound or compounds with similar toxicity, suggesting the high reproducibility of the data and distinguishable toxic signatures in the expression profiles. (Heatmap scale: −0.5:0.5). Top Panel: clusters of genes; left panel: cluster of profiles. FIGS. 1B and 1C show grouping of treatments with the same compound or compounds with similar toxicity, suggesting the high reproducibility of the data and distinguishable toxic signatures in the expression profiles. (Heatmap scale: −0.5:0.5)

FIG. 2. Diagram for ab initio prediction of hepatotoxicity based on transcriptional profiles.

FIG. 3. Hepatotoxicity score was estimated based on five clinical chemistry (CC) measurements, including total bilirubin, ALP, ALT, AST and cholesterol. For individual CC, liver damage was determined by mean and standard deviation (SD). Liver damage, revealed by individual CCs, is indicated by a dark dot. The hepatotoxic score was the weighted sum of normalized individual CC scores. To avoid overinfluence by outliers in the transformation process, sigmoidal normalization was utilized for individual CCs. The weight for each normalized CC score was adjusted according to clinical experience. In particular, the ALT(3) and AST(1.5) were given higher weight than total bilirubin(1). Due to the low specificity of ALP, lower weight was assigned to ALP(0.5). Cholesterol was only included when cholesterol and any one of the other four CCs were abnormal. Liver damage is indicated by a circle when the hepatotoxicity score is greater than −0.25. Among the 267 treated rats, 44 cases of liver damage were identified by individual CCs. Hepatotoxicity score identified 40 of the 44 with liver damage and 3 false positives. x-axis: individual rats tested.

FIG. 4. Optimization of threshold for liver abnormality based on hepatotoxicity score. The threshold of hepatotoxicity score for liver damage was selected so that it revealed the minimal number of false positives and false negatives.

FIG. 5A. Liver damage in the high dose treatment group based on either individual clinical chemistry measurement or hepatotoxicity score is indicated by a black box. Results of the first half of the 148 rats tested: liver damage revealed by individual CC is shown in the combined CCs row (column). Liver damage revealed by hepatotoxicity score is indicated in the hepatotoxicity score row (column). 2 false positives, illustrated by (*), 0 false negatives, illustrated by (\hat{ }) are shown in the high dose treatment group. FIG. 5B. Results of the second half of the 148 rats tested: liver damage revealed by individual CC is shown in the combined CCs row (column). Liver damage revealed by hepatotoxicity score is indicated in the hepatotoxicity score row (column). 1 false positive, illustrated by (*), 1 false negative, illustrated by (\hat{ }) are shown in the high dose treatment group. FIG. 5C. Liver damage in low dose group and non-hepatotoxin treated group based on either individual clinical chemistry or hepatotoxicity score is demonstrated. Total cases of liver damage revealed by individual CC are shown in the combined CCs row (column). Liver damage revealed by hepatotoxicity score is indicated in the hepatotoxicity score row (column). 3 false negatives are seen in the low dose and non-hepatotoxicin group.

FIGS. 6A and 6B. Model for hepatotoxicity score prediction was trained and optimized with 212 expression profiles and their associated hepatotoxicity scores. 238 reporter genes or ESTs, selected by ANOVA (p<0.0000001), were utilized for the prediction. Multi-layer neural network was employed to model the hepatotoxicity score. The optimal model structure was determined by the cross-validating sampling approach. In particular, 80% of the 212 profiles were randomly chosen and utilized as the training set and the rest of 20% were used to determine the estimated error associated with certain neural network structure. The predicted hepatotoxicity scores (squares) from the optimal model and the expected scores (dots) for 212 expression profiles are demonstrated in the top panel (FIG. 6A). The estimated error for prediction from the training set was 0.08. The accuracy and generality of the trained model was examined by another set of 54 expression profiles. The predicted hepatotoxicity scores (squares) from the trained model and the expected scores (dots) for 54 expression profiles were demonstrated in the bottom panel (6B). The estimated error from the validating set was 0.64 and was significantly different from estimated error based on the random data set, indicating that the model can be generalized. The false positive and false negative liver damage predictions were determined by the same threshold, −0.25. 6 false positives, revealed by tip down triangle, and 1 false negative, revealed by tip up triangle, are detected from 54 prediction. The accuracy of the prediction is 88%.

FIG. 7A. Evaluation of sensitivity and specificity of the trained model with the 212 transcriptional profiles in the training data set. The first 60 and the remaining 156 expression profiles and the associated hepatotoxicity scores for the training data set are demonstrated in FIGS. 7A and 7B, respectively. The regulation of 238 reporter genes (left panel) revealed a major pattern associated with liver damage (EP, expected positive) (Heatmap scale: −1˜1). The hepatotoxicity score (dark dots) and the predicted score (line) are shown in the middle panel. The threshold for liver damage is indicated by a dashed line. The expected positives (EP) based on individual CC, and predictive positives (PP) based on hepatotoxicity scores, are illustrated by black boxes in the right panel. The false positive (FP) and false negative (FN) liver damage predictions are also indicated by black boxes in the right panel. The compound, dose and animal identity among the false positive predictions and false negative predictions are revealed by label in bold and italic, respectively. FIG. 7B. Evaluation of sensitivity and specificity of the trained model with the 212 transcriptional profiles in the training data set. The first 60 and the remaining 156 expression profiles and the associated hepatotoxicity scores for the training data set are demonstrated in FIGS. 7A and 7B, respectively. The regulation of 238 reporter genes (left panel) revealed a major pattern associated with liver damage (EP, expected positive) (Heatmap scale: −1˜1). The hepatotoxicity score (dark dots) and the predicted score (line) are shown in the middle panel. The threshold for liver damage is indicated by a dashed line. The expected positives (EP) based on individual CC, predictive positives (PP) based on hepatotoxicity scores are illustrated by black boxes in the right panel. The false positive (FP) and false negative (FN) liver damage predictions are also indicated by black boxes in the right panel. The compound, dose and animal identity among the false positive predictions and false negative predictions are revealed by label in bold and italic, respectively.

FIG. 8. The expression profiles and the associated hepatotoxicity scores for the validating set of 54 expression profiles are demonstrated. The regulation of 238 reporter genes(left panel) revealed a major pattern associated with liver damage (EP, expected positive) (Heatmap scale: −1˜1). The hepatotoxicity score (dark dots) and the predicted score (line) are shown in the middle panel. The threshold for liver damage is indicated by a dashed line. The expected positive (EP) based on individual CC, and predictive positive (PP) based on hepatotoxicity scores, are illustrated by black boxes in the right panel. The false positive (FP) and false negative (FN) liver damage predictions are also indicated by black boxes in the right panel. Detailed information about the compound, dose and animal identity among the missed predictions are labeled in italic and bold for false negative and false positive predictions, respectively. Experiments showing similar expression patterns (indicated by >) but subnormal predicted hepatotoxicity score uncovers the source of error associated with the model. More interestingly, pathological changes were observed in 4 of the 6 false positives predicted from gene expression profiles, indicating that these 4 were true positives. This result suggests that the present model is more sensitive in predicting liver damage than individual CCs.

FIG. 9 is a schematic illustration of a multilayer neural network.

FIG. 10 illustrates an exemplary embodiment of a computer system useful for implementing the methods of this invention.

FIG. 11 illustrates expression patterns that are unique for five classes of hepatotoxicity, necrosis, phospholipidosis, steatosis, cholestasis, and hypertrophy due to enzyme induction.

### 5. DETAILED DESCRIPTION OF THE INVENTION

The invention provides methods for characterizing the condition or status of a tissue or organ in a multicellular organism, e.g., an animal, by combining a plurality of clinical measures into a composite clinical score (CCS) and using such a CCS to represent the condition or status of the tissue or organ. The invention provides methods for predicting the condition or status of a tissue or organ in a multicellular organism, e.g., a plant or an animal, based on measurements of a set of cellular constituent markers, e.g., measured expression levels of a set of marker genes. The methods of the invention involve using a machine learning algorithm to build a model for determining a suitable composite clinical score of the tissue or organ based on response profiles comprising measurements of a set of cellular constituent markers. The invention also provides methods for reduction of variable dimensions of response profiles, e.g., by transforming a profile into a feature space of reduced dimension using, e.g., a wavelet transformation. The invention also provides methods for selecting the set of cellular constituent markers whose levels can be used in determining the CCS. The methods of the invention are applicable to any multicellular organisms. For example, the methods are applicable to any animals, including but are not limited to mammals (e.g., non-human mammals, primates, horses, cows, pigs, dogs, cats, sheep, goats, mice, rats, etc.), and, in a preferred embodiment, a human.

### 5.1. Biological State and Cellular Constituent Profile

The state of a cell or other biological sample is represented by cellular constituents (any measurable biological variables) as defined in Section 5.1.1, infra. Those cellular constituents vary in response to perturbations, or under different conditions. The measured signals can be measurements of such cellular constituents or measurements of responses of cellular constituents.

### 5.1.1. Biological State

As used herein, the term “biological sample” is defined to include any cell, tissue, organ or multicellular organism. A biological sample can be derived, for example, from cell or tissue cultures in vitro. Alternatively, a biological sample can be derived from a living organism or from a population of single cell organisms. In preferred embodiments, the biological sample comprises a living cell or organism.

The state of a biological sample can be measured by the content, activities or structures of its cellular constituents. The state of a biological sample can be the state of a collection of cellular constituents, which are sufficient to characterize the cells of a tissue or organ for an intended purpose including, but not limited to characterizing the effects of a drug or other perturbation. The term “cellular constituent” is defined in this disclosure to encompass any kind of measurable biological variable. The measurements and/or observations made on the state of these constituents can be of their abundances (i.e., amounts or concentrations in a biological sample) e.g., of mRNA or proteins, or their activities, or their states of modification (e.g., phosphorylation), or other measurements relevant to the biology of a biological sample. In various embodiments, this invention includes making such measurements and/or observations on different collections of cellular constituents, such as gene products (protein, RNA or cDNA derived therefrom) corresponding to (or encoded by) a gene. These different collections of cellular constituents are also called herein aspects of the biological state of a biological sample.

One aspect of the biological state of a biological sample (e.g., a cell or cell culture) usefully measured in the present invention is its transcriptional state. In fact, the transcriptional state is the currently preferred aspect of the biological state measured in this invention. The transcriptional state of a biological sample includes the identities and abundances of the constituent RNA species, especially mRNAs, in the cell under a given set of conditions. Preferably, a substantial fraction of all constituent RNA species in the biological sample are measured, but at least a sufficient fraction is measured to characterize the action of a drug or other perturbation of interest. The transcriptional state of a biological sample can be conveniently determined by, e.g., measuring cDNA abundances by any of several existing gene expression technologies. One particularly preferred embodiment of the invention employs DNA arrays for measuring mRNA or transcript level of a large number of genes. The other preferred embodiment of the invention employs DNA arrays for measuring expression levels of a large number of genes or exons in the genome of an organism.

Another aspect of the biological state of a biological sample usefully measured in the present invention is its translational state. The translational state of a biological sample includes the identities and abundances of the constituent protein species in the biological sample under a given set of conditions. Preferably, a substantial fraction of all constituent protein species in the biological sample is measured, but at least a sufficient fraction is measured to characterize the action of a drug of interest. As is known to those of skill in the art, the transcriptional state is often representative of the translational state.

Still another aspect of the biological state of a biological sample is its small molecule state, e.g., metabolic state. The small molecule state of a biological sample comprises identities and abundances of small molecules present in a cell. Small molecules refer to molecules of molecular weights of less than about 5000, including but are not limited to sugars, fatty acids, amino acids, nucleotides, intermediates of cellular processes, e.g., intermediates of metabolic and signaling pathways. Other aspects of the biological state of a biological sample are also of use in this invention. For example, the activity state of a biological sample, as that term is used herein, includes the activities of the constituent protein species (and also optionally catalytically active nucleic acid species) in the biological sample under a given set of conditions. As is known to those of skill in the art, the translational state is often representative of the activity state.

This invention is also adaptable, where relevant, to “mixed” aspects of the biological state of a biological sample in which measurements of different aspects of the biological state of a biological sample are combined. For example, in one mixed aspect, the abundances of certain RNA species and of certain protein species, are combined with measurements of the activities of certain other protein species. Further, it will be appreciated from the following that this invention is also adaptable to other aspects of the biological state of the biological sample that are measurable.

The biological state of a biological sample (e.g., a cell or cell culture) is represented by a profile of some number of cellular constituents. Such a profile of cellular constituents can be represented by the vector S,
S=[S₁, . . . Sᵢ, . . . Sₖ] (1)
where Sᵢ is the level of the i'th cellular constituent, for example, the transcript level of gene i, or alternatively, the abundance or activity level of protein i. In preferred embodiments, k is more than 2, preferably more than 10, more preferably more than 100, still more preferably more than 1000, still more preferably more than 10,000, still more preferably more than 25,000, still more preferably more than 50,000, and most preferably more than 100,000.

In some embodiments, cellular constituents are measured as continuous variables. For example, transcriptional rates are typically measured as number of molecules synthesized per unit of time. Transcriptional rate may also be measured as percentage of a control rate. However, in some other embodiments, cellular constituents may be measured as categorical variables. For example, transcriptional rates may be measured as either “on” or “off”, where the value “on” indicates a transcriptional rate above a predetermined threshold and value “off” indicates a transcriptional rate below that threshold.

### 5.1.2. Biological Responses

The responses of a biological sample to a perturbation, i.e., under a condition, such as the application of a drug, can be measured by observing the changes in the biological state of the biological sample. For example, the responses of a biological sample can be responses of a living cell or organism to a perturbation, e.g., application of a drug, a genetic mutation, an environmental change, and so on, to the living cell or organism. A response profile is a collection of changes of cellular constituents. In the present invention, the response profile of a biological sample (e.g., a cell or cell culture) to the perturbation m is defined as the vector v^{(m)}:
v^{(m)}[v₁^{m}, . . . vᵢ^{(m)}, . . . vₖ^{(m)}] (2)

Where vᵢ^{m} is the amplitude of response of cellular constituent i under the perturbation m. In some particularly preferred embodiments of this invention, the biological response to the application of a drug, a drug candidate or any other perturbation, is measured by the induced change in the transcript level of at least 2 genes and/or proteins, preferably more than 10 genes and/or proteins, more preferably more than 100 genes and/or proteins, still more preferably more than 1000 genes and/or proteins, still more preferably more than 10,000 genes and/or proteins, still more preferably more than 25,000 genes and/or proteins, still more preferably more than 50,000 genes and/or proteins, and most preferably more than 100,000 genes and/or proteins. In another preferred embodiment of the invention, the biological response to the application of a drug, a drug candidate or any other perturbation, is measured by the induced change in the expression levels of a plurality of exons in at least 2 genes and/or proteins, preferably more than 10 genes and/or proteins, more preferably more than 100 genes and/or proteins, still more preferably more than 1000 genes and/or proteins, still more preferably more than 10,000 genes and/or proteins, still more preferably more than 25,000 genes and/or proteins, still more preferably more than 50,000 genes and/or proteins, and most preferably more than 100,000 genes and/or proteins. In some embodiments of the invention, the response is simply the difference between biological variables before and after perturbation. In some preferred embodiments, the response is defined as the ratio of cellular constituents before and after a perturbation is applied. In other embodiments, the response may be a function of time after the perturbation, i.e., v(m)=v(m)(t). For example v(m)(t) may be the difference or ratio of cellular constituents before the perturbation and at time t after the perturbation.

In some preferred embodiments, vᵢ^{m} is set to zero if the response of gene i is below some threshold amplitude or confidence level determined from knowledge of the measurement error behavior. In such embodiments, those cellular constituents whose measured responses are lower than the threshold are given the response value of zero, whereas those cellular constituents whose measured responses are greater than the threshold retain their measured response values. This truncation of the response vector is a good strategy when most of the smaller responses are expected to be greatly dominated by measurement error. After the truncation, the response vector v^{(m)} also approximates a ‘matched detector’ (see, e.g., Van Trees, 1968, Detection, Estimation, and Modulation Theory Vol. I, Wiley & Sons) for the existence of similar perturbations. It is apparent to those skilled in the art that the truncation levels can be set based upon the purpose of detection and the measurement errors. For example, in some embodiments, genes whose transcript level changes are lower than two fold or more preferably four fold are given the value of zero.

In some preferred embodiments, perturbations are applied at several levels of strength. For example, different amounts of a drug may be applied to a biological sample to observe its response. In such embodiments, the perturbation responses may be interpolated by approximating each by a single parameterized “model” function of the perturbation strength u. An exemplary model function appropriate for approximating transcriptional state data is the Hill function, which has adjustable parameters a, u₀, and n.

$$H ( u ) = \frac{{a ( u / {u}_{0} )}^{n}}{1 + {( u / {u}_{0} )}^{n}} \qquad ( 3 )$$

The adjustable parameters are selected independently for each cellular constituent of the perturbation response. Preferably, the adjustable parameters are selected for each cellular constituent so that the sum of the squares of the differences between the model function (e.g., the Hill function, Equation 3) and the corresponding experimental data at each perturbation strength is minimized. This preferable parameter adjustment method is well known in the art as a least squares fit. Other possible model functions are based on polynomial fitting, for example by various known classes of polynomials. More detailed description of model fitting and biological response has been disclosed in Friend and Stoughton, Methods of Determining Protein Activity Levels Using Gene Expression Profiles, PCT publication WO 99/59037, which is incorporated herein by reference in its entirety for all purposes.

### 5.2. Clinical or Diagnostic Measures

The condition or status of a tissue or organ in a multicellular organism, e.g., an animal (e.g., a human) or a plant, can be characterized by one or more suitable diagnostic measures. The tissue or organ the condition of which is characterized can be any type of tissue or organ in the organism. For example, in cases of animals, liver, kidney, lung, heart, brain, spleen, muscle, etc., can be characterized by one or more clinical or diagnostic measures. As used herein, the condition or status of a tissue or organ includes any aspect of the physiological and/or functional condition or status of the tissue or organ, e.g., whether the tissue or organ is normal or diseased, such as subject to a disorder or damage or infection, etc. As a non-limiting example, the condition or status of a tissue or organ, e.g., liver, can be the degree of tissue or organ damage in an animal treated with a drug. The condition or status of a tissue or organ may reflect the biological state of a type of cell associated with the tissue or organ if the tissue or organ comprises primarily a single type of cells. The condition or status of a tissue or organ may also reflect the biological states of a plurality of different types of cells associated with the tissue or organ if the tissue or organ comprises more than one type of cell. The condition or status of a tissue or organ may also reflect changes in cell types, e.g., change of normal cells to one or more abnormal types of cells, e.g., cancerous, hyperplastic, dysplastic, or metaplastic cells.

The clinical or diagnostic measures include any observational and/or measurable variables which can be used to indicate the condition or status of the tissue or organ. As non-limiting examples, these variables can be acquired by health history, physical examination, laboratory test, and/or histopathological examination, e.g., biopsy. Given the tissue or organ and the aspect of the condition or status of the tissue or organ of interests, an ordinary skilled person in the art will be able to determine the appropriate clinical or diagnostic measures for use. As used herein, the clinical or diagnostic measures include but are not limted to those measures commonly used in the art to assess the condition or health status of the tissue or organ.

Laboratory tests often provide the most reliable and widely used clinical or diagnostic measures. Laboratory tests include but are not limited to blood tests, urine tests, etc. An organ or tissue in an animal releases various molecules into the blood and/or other body fluid, e.g., urine. The identities and/or concentrations of such molecules may correlate with the condition or status of the tissue or organ which releases them. They may reflect different aspects of the condition or status of the tissue or organ. For example, to characterize the condition or status of liver, e.g., the degree of liver damage due to a drug or the degree of a liver disease such as hepatitis B or C, serum levels of alanine aminotransferase (ALT), aspartate aminotransferase (AST), alkaline phosphatase (ALP), bilirubin (Tbil), cholesterol (Chol), gamma-glutamyltranspeptidase (GGT), albumin, globulins, prothrombin time, etc., are often measured (see, e.g., Fogy, 1999, Clinical Chemistry: Principles, Procedures, Correlations, Lippincott Wiliams & Wilkines; and Zimmerman, 1999, Hepatotoxicity: The adverse effects of drugs and other chemicals on the Liver, Lippincott Williams & Wilkins).

ALT is an enzyme specifically produced in the hepatocyte, the major cell type in the liver. When liver cells are damaged, ALT is released into the bloodstream. Thus, the level of ALT in the blood correlates with the degree of hepatocyte damage and/or death. Many types of liver diseases, e.g., hepatitis, cause hepatocyte damage that can lead to elevated serum ALT level. Liver cell damage or death resulting from other causes, such as shock or drug toxicity can also lead to elevated serum ALT level. In preferred embodiments, the normal range of the ALT level is determined by the average and standard deviation of animals in a normal or control group, e.g., animals not having a condition which causes an abnormal ALT level. In one embodiment, a level within +/−two standard deviations of the averaged level of normal animals is deemed to be normal.

AST is another enzyme whose level in serum may indicate liver damage. However, AST is not specific for liver damage or disease as it is also produced in the muscles and can be elevated in other conditions. In many cases of liver damage, the ALT and AST levels are elevated roughly in a 1:1 ratio. However, in certain other conditions, such as alcoholic hepatitis, the elevation in the serum AST level may be higher than the elevation in the serum ALT level. In preferred embodiments, the normal range of the AST level is determined by the average and standard deviation of animals in a normal or control group, e.g., animals not having a condition which causes an abnormal AST level. In one embodiment, a level within +/− two standard deviations of the averaged level of normal animals is deemed to be normal.

ALP includes a family of related enzymes produced in the bile ducts, intestine, kidney, placenta and bone. An elevation in the level of serum ALP, especially in the setting of normal or only modestly elevated ALT and AST levels, may suggest damage or disease of the bile ducts. Serum ALP level can be markedly elevated in bile duct obstruction or in bile duct diseases such as primary biliary cirrhosis or primary sclerosing cholangitis. ALP is also produced in intestine, kidney, placenta and bone, and thus its blood level may also be increased due to abnormalities in these tissues or organs. In preferred embodiments, the normal range of the ALP level is determined by the average and standard deviation of animals in a normal or control group, e.g., animals not having a condition which causes an abnormal ALP level. In one embodiment, a level within +/−two standard deviations of the averaged level of normal animals is deemed to be normal.

GGT is another enzyme produced in the bile ducts whose level, like ALP, may be elevated in the serum of an animal with bile duct damage or diseases. Thus, elevation in serum GGT, especially along with elevations in ALP, often indicates bile duct damage or disease. The measurement of GGT is extremely sensitive. Thus, elevation of its level can be readily determined in many liver diseases and even sometimes in normal individuals. In preferred embodiments, the normal range of the GGT level is determined by the average and standard deviation of animals in a normal or control group, e.g., animals not having a condition which causes an abnormal GGT level. In one embodiment, a level within +/− two standard deviations of the averaged level of normal animals is deemed to be normal.

Bilirubin is the major breakdown product of old red blood cells. It is removed from the blood by the liver, chemically modified by a process called conjugation, secreted into the bile, passed into the intestine and to some extent reabsorbed from the intestine. Bilirubin concentrations are elevated in the blood either by increased production, decreased uptake by the liver, decreased conjugation, decreased secretion from the liver or blockage of the bile ducts. In cases of increased production, decreased liver uptake or decreased conjugation, the level of unconjugated or the so-called indirect bilirubin will be elevated. In cases of decreased secretion from the liver or bile duct obstruction, the level of conjugated or the so-called direct bilirubin will be elevated. Many different liver diseases, as well as conditions other than liver diseases (e.g. increased bilirubin production as a result of enhanced red blood cell destruction), can cause elevation in the serum bilirubin concentration. Most liver diseases cause impairment in bilirubin secretion from liver cells, which leads to increased levels of direct bilirubin in the blood. In chronic, acquired liver diseases, the serum bilirubin concentration is usually normal until a significant amount of liver damage has occurred and cirrhosis is present. In acute liver diseases, the bilirubin is usually increased in accordance with the severity of the damage. In bile duct obstruction, or diseases of the bile ducts such as primary biliary cirrhosis or sclerosing cholangitis, the ALP and GGT levels are often elevated along with the direct bilirubin level. Total bilirubin (Tbil) tests measure the amount of bilirubin in the bloodstream. In preferred embodiments, the normal range of the direct or total bilirubin level is determined by the average and standard deviation of animals in a normal or control group, e.g., animals not having a condition which causes an abnormal bilirubin level. In one embodiment, a level within +/− two standard deviations of the averaged level of normal animals is deemed to be normal.

Albumin is the major protein that circulates in the bloodstream. Albumin is synthesized by the liver and secreted into the blood. Low serum albumin concentrations indicate poor liver function. The serum albumin concentration is usually normal in chronic liver diseases until cirrhosis and significant liver damage is present. Albumin levels can be low in conditions other than liver diseases, e.g., in certain kidney diseases. In preferred embodiments, the normal range of the albumin level is determined by the average and standard deviation of animals in a normal or control group, e.g., animals not having a condition which causes an abnormal albuminn level. In one embodiment, a level within +/− two standard deviations of the averaged level of normal animals is deemed to be normal.

The prothrombin time is a blood clotting test for determining the blood concentrations of some of the clotting factors made by the liver are low. The normal time needed for blood to clot is between 10 and 15 seconds. In chronic liver diseases, the prothrombin time can be prolonged when cirrhosis is present and the liver damage is fairly significant. In acute liver diseases, the prothrombin time can be prolonged with severe liver damage but may return to normal as the patient recovers. Prothrombin time can also be prolonged in certain non-liver disorders.

The concentration or level of major serum proteins can also be used for determining liver damage or disease. The major proteins in the serum are separated by electrophoresis and their concentrations determined. The major types of serum proteins whose concentrations are measured in this test are albumin and globulin including alpha-globulins, beta-globulins and gamma-globulins. In cirrhosis, the level of albumin may be decreased while the level of gamma-globulin elevated. Gamma-globulin may also be significantly elevated in some types of autoimmune hepatitis.

Liver condition can also be evaluated by histopathological analysis. Biological variables or parameters that can be measured in a histopathological analysis and used for characterizing the status of liver are known to a skilled person in the art (see, e.g., Waring et al., 2001, Toxicology and Applied Pharmacology 175: 28-42).

### 5.3. Methods of Characterizing Tissue or Organ Condition

The present invention provides methods for characterizing the condition or status of a tissue or organ in an animal. The methods of the invention comprise determining a composite clinical score (CCS) based on a plurality of clinical measures of the tissue or organ of interests. Preferably, the composite clinical score is a continuous score. Such a composite clinical score can be used to quantitatively characterize the condition or status of the tissue or organ. As an exemplary embodiment, a hepatotoxicity score is provided to measure the degree of liver damage due to the toxicity of a drug. In the disclosure, the methods of the invention are often illustrated using the exemplary embodiments involving drug induced liver damage and the hepatotoxicity score. It will be apparent to one skilled in the art that the methods are applicable to composite clinical scores for characterizing the conditions or statues of other tissues or organs.

As described in Section 5.2, the condition or status of a tissue or organ can be evaluated by a variety of clinical or diagnostic measures. A continuous score which combines information from a plurality of clinical measures provides a better means for characterizing the status of the tissue or organ. For example, in cases of liver damage or diseases, alanine aminotransferase (ALT) and aspartate aminotransferase (AST) in plasma can be used as indicators for hepatocellular injury with a certain degree of specificity. Direct and total bilirubin (Tbil) measurements in plasma can be utilized to monitor cholestasis. In addition, the histopathological approach can provide qualitative evaluation of liver injuries at a cellular level. For example, several types of cellular change, such as necrosis, hypertrophy, steatosis and cholestasis, have been observed from drug induced liver damage. However, due to the complexity of liver injury, the severity of liver damage cannot be sufficiently described by any single one of those indicators. The severity of liver damage can be evaluated more accurately by combining more than one of these clinical measures, e.g., for prediction of drug hepatotoxicity.

In one embodiment, k clinical measures are selected to construct a composite clinical score, e.g., for each of M individual animals. For example, k can be 2, 3, 5 or 10. In a preferred embodiment, the composite clinical score of the jth animal is a linear combination of the k clinical measures

$$CCS ( j ) = {\sum }_{i = 1}^{k} {α}_{i , j} \cdot {d}_{i , j} \qquad ( 4 )$$

where di,j is the ith clinical measure the jth animal, α_{i,j} is the coefficient of the ith clinical measure the jth animal. Other suitable mathematical combination of the k clinical measures can be used in the present invention.

The k clinical measures can also be converted before used in the construction of the composite clinical score. In a preferred embodiment of the invention, the absolute value each of the k clinical measures for each of the M individual animals is first converted into a distance between a reference value and the actual measured value, i.e., a deviation of the actual measured value from the reference value. The reference value is determined based on the status or condition to be evaluated. In one embodiment, when degree of abnormality, e.g., degree of a disease, of a tissue is to be determined, the reference value can be the value of the clinical measure associated with a normal tissue. Alternatively, the reference value can also be the value of the clinical measure associated with a particular level of abnormality, e.g., a particular level of a disease. In the disclosure, for simplicity reasons, embodiments using a reference value which is associated with a normal tissue, i.e., a normal value, are often described. It will be apparent to one skilled in the art that other different types of reference values can also used in the present invention. Any suitable form of distance representing the deviation can be used for this purpose. In one embodiment, the distance is determined according to Equation (5)

$${D}_{i , j} = \frac{{x}_{i , j} - {μ}_{i , 0}}{{σ}_{i , 0}} \qquad ( 5 )$$

where D_{i,j} is the ith converted clinical measure of the jth animal, x_{i,j} is the ith clinical measure of the jth animal, μ_{i,0} is a reference value of the ith clinical measure, e.g., a value measured in a control sample, and σ_{i,0} is standard deviation of the ith clinical measure, and where i=1, 2, . . . , k, and j=1, 2, . . . , M.

In another embodiment, each of the k clinical measures or converted clinical measures are sigmoidally normalized with different range of linear transformation region. The sigmoidal transformation converts data non-linearly into the range of {−1˜1}. This transformation retain outliers (e.g., abnormal values indicating significant deviation from normal values) without compressing the most commonly occurring values close to the threshold level in treated groups. Preferably, the range of linear transformation is adjusted for each clinical measure by taking into account of the different dynamic ranges among the different clinical measures. More preferably, the sensitivity and specificity of the clinical measure are taken into account in determining the range of linear transformation. Increasing the range for linear transformation may prevent more sub-threshold values from being compressed. In a preferred embodiment, the sigmoidal transformation is carried out according to the Equation (6)

$$\begin{matrix}{D}_{i , j}^{′} = \frac{1 - {ⅇ}^{- {α}_{i , j}}}{1 + {ⅇ}^{- {α}_{i , j}}} \\ where & ( 6 ) \\ {α}_{i , j} = \frac{{D}_{i , j} - {\overline{D}}_{i}}{{c}_{i} \cdot Std ( {\overline{D}}_{i} )} & ( 7 )\end{matrix}$$

where D_{i,j} is the ith converted clinical measure of the jth animal, \overline{D}ᵢ is a reference value, e.g., the normal value, of the ith clinical measure, e.g., a value measured from an animal whose tissue or organ is normal, and std(\overline{D}ᵢ) is the standard derivation of \overline{D}ᵢ, cᵢ is a constant associated with the ith clinical measure, and i=1, 2, . . . , k, and j=1, 2, . . . , M. In a preferred embodiment, \overline{D}ᵢ is the average of measurements of the ith clinical measure measured from animals in a normal or control group. In another embodiment, when effect of one or more drugs on the tissue or organ is concerned, \overline{D}ᵢ is the average of measurements of the ith clinical measure measured from animals that are not subject to the drug or drugs. Other suitable definition of α_{i,j} can also be used. A skilled person in the art will be able to select an appropriate definition of α_{i,j}.

The transformed clinical measures can then be used in construction of the composite clinical score. In a preferred embodiment,

$$CCS ( j ) = {\sum }_{i = 1}^{k} {β}_{i , j} \cdot {D}_{i , j}^{′} \qquad ( 8 )$$

Preferably, when transformed clinical measures are used in construction of the CCS by linear combination, the coefficients {β_{i,j}}, i=1, 2, . . . , k; j=1, 2, . . . , M, can be determined by a method which takes into account of the different dynamic ranges among the different clinical measures. More preferably, the sensitivity and specificity of a clinical measure are taken into account in determining its coefficient. In one embodiment, clinical measures with higher sensitivity and specificity are given a higher weight.

In one embodiment, to reflect the degree of liver damage, the invention provides a hepatotoxicity score (HS) which integrates several traditional clinical measurements into a continual index to characterize liver damage. In a preferred embodiment, the hepatotoxicity score utilizes five clinical chemistry indicators, specifically, ALT, AST, Tbil, ALP and Chol to measure the degree of liver injury resulting from numerous aspects of cellular damage. These clinical measures are converted into a distance between the actual measured value and the normal value according to Eq. 3. The converted clinical measures are then sigmoidally normalized according to Eqs. 6 and 7. In a preferred embodiment, ALT, the most sensitive indicator of liver cell damage, values within 3× standard deviations, instead of 1×, of the average were mapped to the most linear region of the sigmoid, i.e., for ALT c=3, and for all other measures, c=1. In a preferred embodiment, the hepatotoxicity score (HS) is defined according Eq. (9):

$$HS = {D}_{Tbil}^{′} ( if Tbil is abnormal ) + \\ 0.5 {D}_{ALP}^{′} + 3 {D}_{ALT}^{′} + 1.5 {D}_{AST}^{′} + \\ 0.3 {D}_{Chol}^{′} ( if both Chol and at \\ least one other clinical measure are abnormal ) \qquad ( 9 )$$

where contribution from Tbil is zero if Tbil is normal, and the contribution from Chol is zero unless both Chol and at least one of the other clinical measures are abnormal. In one embodiment, a level for any one of the clinical measure in Eq. 9 is considered abnormal if it is beyond +/− two standard deviations of the averaged level in normal or control animals.

In one embodiment, to evaluate the constructed CCS, the number of false positives and false negatives detected by the CCS can be identified by comparing with results from or or more individual clinical measures. In one embodiment, all individual clinical measures are pooled together to form a single ‘gold standard.’ In such an embodiment, an animal is said to be positive for liver damage if at least one of the individual clinical measures is positive for liver damage. The results from the CCS are then compared with such a gold standard. A false positive by the CCS indicates that a positive is identified by the CCS but not the gold standard, whereas a false negative by the CCS indicates that a negative is identified by the CCS but identified as a positive by the gold standard.

In one embodiment, a threshold for the CCS to indicate normal condition of the tissue or organ characterized by the CCS is determined. The threshold may be used to distinguish the condition or status of the tissue or organ the CCS characterizes as normal or abnormal. For example, in cases of liver damage or disease, a threshold for HS can be determined to classify livers as normal or damaged or diseased. Such a threshold can also be used for classification of perturbations to the tissue or organ of interest based on their effect on the condition of the tissue or organ, e.g., for classification of hepatotoxins.

In a preferred embodiment, the threshold for the CCS is chosen such that the false negative rate is minimized using a set of animals having both normal and abnormal conditions. In another preferred embodiment, the threshold for the CCS is chosen such that the false positive rate is minimized. In still another preferred embodiment, threshold for the CCS is chosen such that the rate of total misclassification, i.e., the sum of false positives and false negatives, is minimized. A combination of any two or more of the above criteria can also be used.

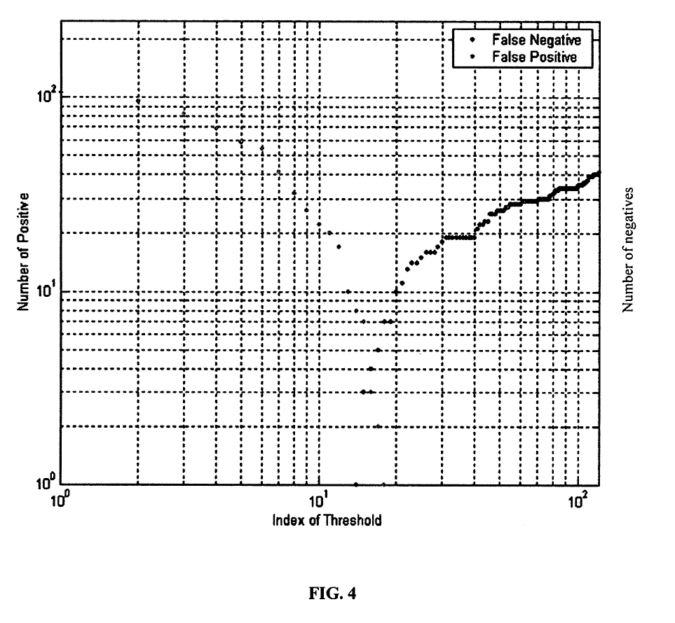

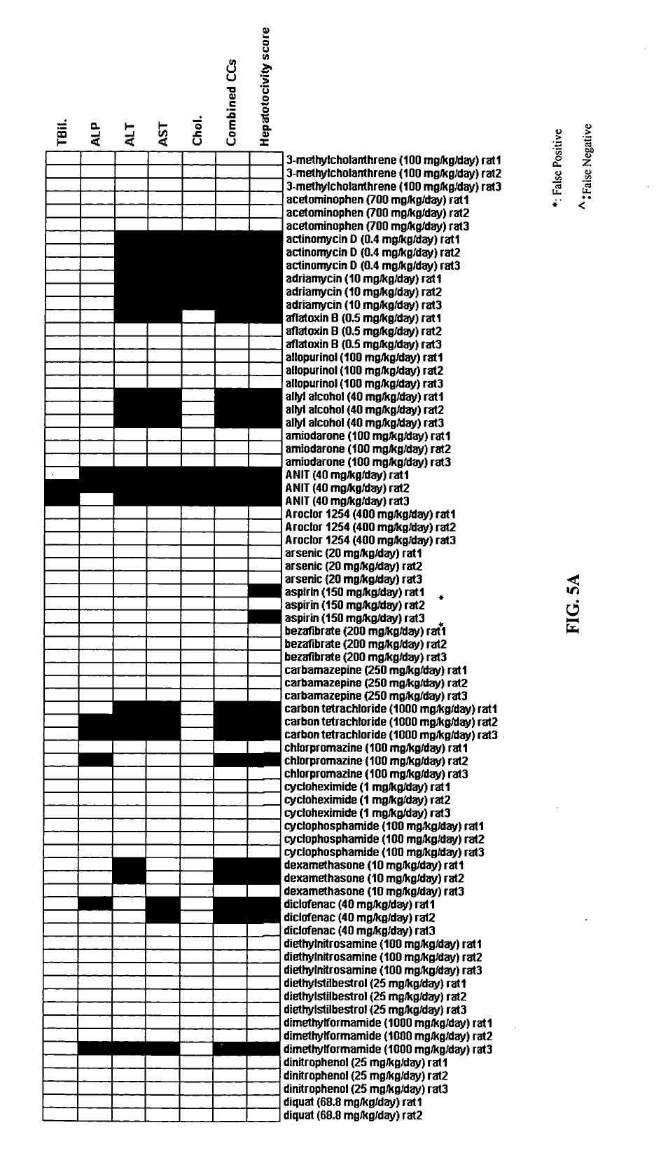

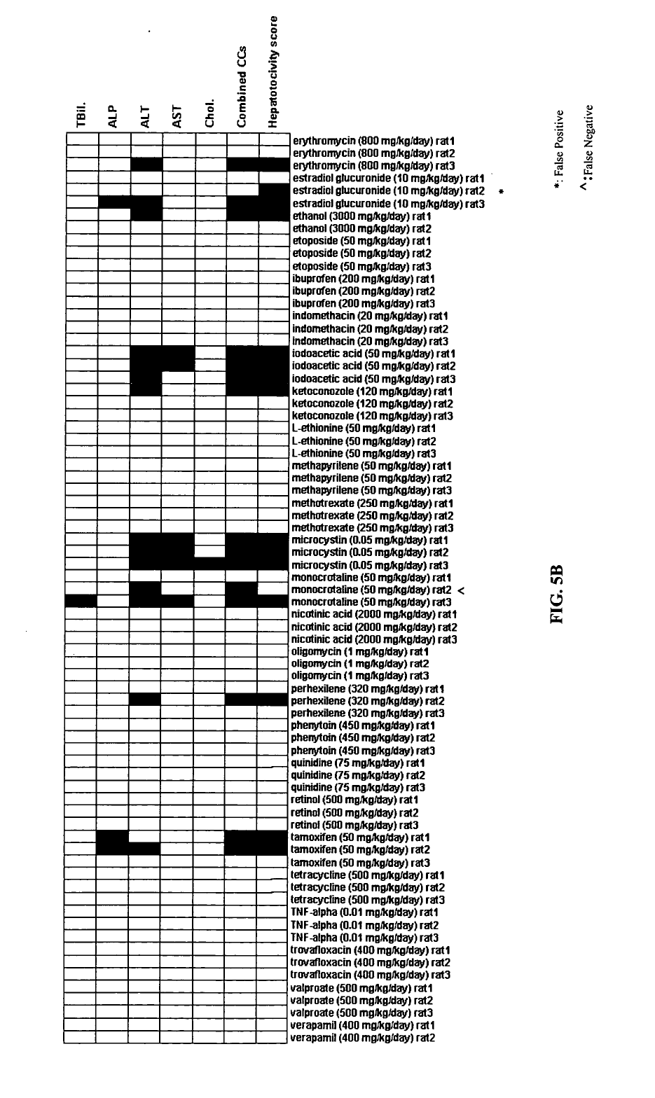

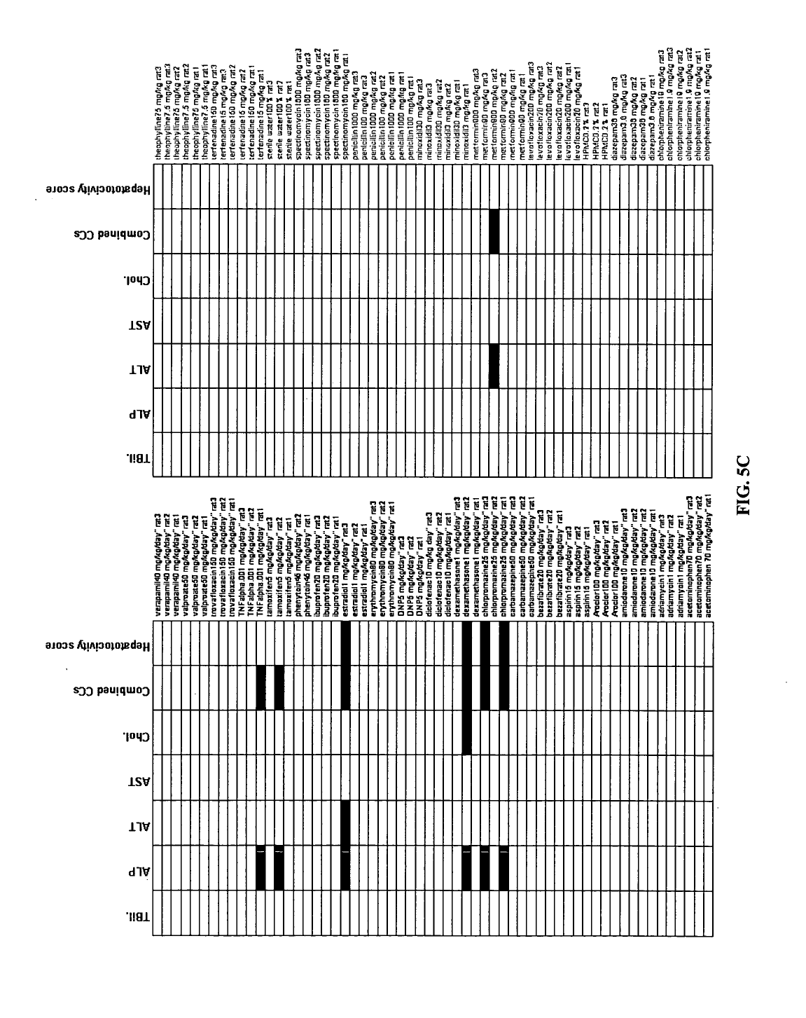

Preferably, the sensitivity and accuracy of the CCS are evaluated to determine the rate of misclassification, e.g., false positive and/or false negative rates. In one embodiment, the CCS is compared with individual clinical measure, including both clinical measures used in constructing the CCS and additional clinical measures, e.g., histopathological data. In a specific embodiment, in cases of liver damage or diseases, the threshold for HS as defined by Eq. 9 is chosen by minimizing the false negatives and the sum of false positives and false negatives (FIG. 4) in a set of animals whose liver conditions have been determined. In one embodiment, the liver condition of the animals are determined by biopsy. In a particularly preferred embodiment, a value of −0.25 is selected as liver abnormality threshold for the HS. The HS has higher sensitivity and specificity than any one of the component clinical measures. For example, comparing liver damage revealed by the hepatotoxicity score and liver damage indicated by individual clinical chemistry measurement, 90.9% positives are detected by using hepatotoxicity score, whereas only 56.8% positives are detected by using AST, 79.5% positives are detected by using ALT, 36% positives are detected by using ALP, 6.8% positives are detected by using Tbil. and 25% are detected by using Chol. (FIGS. 5A-5C). The increasing sensitivity of the hepatotoxicity score suggests the combination power from individual clinical chemistry indicators because those individual indicators, such as ALT and Tbil, are most sensitivity to only a certain aspect of liver damage. For example, in the high dose experiment group, liver damage is only reported by ALT in the No.2 rat that received perhexilene (320 mg/kg/day). Another case of liver abnormality is only detected by AST in the No. 1 rat received ethanol (3000 mg/kg/day) treatment. Such abnormality would not be detected with either AST or ALT alone. However, as the combination of all five clinical chemistry indicators, the hepatotoxicity score is more sensitive in detecting liver abnormality.

To further evaluate the accuracy of the hepatotoxicity score, the histopathological examination results from the four false negatives and three false positives detected by the hepatotoxicity score have been investigated (FIGS. 5A-B and 5C). Among the four false negatives, one received metformin, a non-hepatotoxicin (900 mg/kg/day), two received low dose hepatotoxins, TNF-alpha at 0.01 mg/kg/day and tamoxifen at 5 mg/kg/day and one received high dose hepatotoxin, monocrotaline at 50 mg/kg/day treatment. Histopathology exam reported no observable liver abnormality in those four samples. This observation suggests that the specificity of the hepatotoxicity score is higher than that of the individual clinical chemistry measurements. Among the three false positive cases, all of them belong to high dose hepatotoxin-treated group. Specifically, the positives detected by the hepatotoxicity score, but not by any of the clinical chemistry indicators, include the estradiol glucuronide (10 mg/kg/day) and aspirin (150 mg/kg/day) treated groups. Cellular abnormality has been reported in both treatment groups. Among the three estradiol glucuronide treated rats, the other two also showed liver injury by ALT and ALP. The evidence from histopathology examination and clinical chemistry measurements for other members of these treated rats suggests that the hepatotoxicity score is sensitive enough to reveal mild degrees of liver injury that cannot be detected by any single clinical chemistry measurement. Hence, the hepatotoxicity score is a comprehensive indicator of the degree of liver damage with reliable specificity and sensitivity. It can be utilized to estimate the degree of liver damage in response to compounds.

### 5.4. Methods of Predicting Tissue Condition Based on Measured Levels of Cellular Constituent Markers

The invention provides methods for predicting tissue or organ condition based on measurements of a set of cellular constituent markers, e.g., measured expression levels of a set of marker genes. The methods of the invention involves using a machine learning algorithm to build a model for determining a composite clinical score of the tissue or organ, e.g., a hepatotoxicity score, using the expression levels of the set of markers. The invention also provides methods for reduction of variable dimension of response profiles, e.g., by transforming a profile into a feature space of reduced dimension using, e.g., a wavelet transformation. The invention also provides methods for selecting the set of cellular constituent markers, e.g., marker genes, whose expression levels can be used in determining the CCS. For example, the set of cellular constituent markers can include cellular constituents that are known to change in response a perturbation to the cells. The measurements of the cellular constituent markers can be transformed measurements obtained using an appropriate transformation, see, e.g., Weng, U.S. patent application Ser. No. 10/354,664, filed on Jan. 30, 2003, which is incorporated by reference herein in its entirety.

### 5.4.1. Compendium and Cellular Constituent Markers

In preferred embodiments, the present invention is practiced using a database or “compendium” of expression profiles or biological response profiles. The compendium used in the methods of the present invention may be a compendium of response profiles of a type of tissue or organ of a multicellular organism, e.g., an animal, in one or more individuals under different conditions, e.g., under treatment of different drugs, having different levels of a disease, etc. Biological variables associated with each response profile, including but are not limited to identity of the animal from whom the response profile is derived, variables characterizing the condition of the animal, e.g., identity and dosage of a drug used to treat the animal, the degree or level of a disease, etc.; one or more clinical measures and/or one or more composite clinical scores determined based on such clinical measures, e.g., clinical measures or composite clinical score as described in Sections 5.2 and 5.3, are also contained in the compendium. The compendium can comprise a plurality of expression profiles of the tissue or organ under a large number of different conditions, e.g., more than 50, 100, 1,000, or 10,000 profiles, each under a different condition.

The compendium can be constructed based on a plurality of different aspects of a tissue to be evaluated. Such a compendium can be used for, e.g., developing a model for evaluating if the tissue or organ is normal or is abnormal due to one of a plurality of diseases and/or disorders. For each different aspect of the tissue or organ, expression profiles corresponding to a plurality of different levels or degrees may also be included in the compendium. The compendium can also be constructed based on a particular aspect of the tissue to be evaluated. Such a compendium can be used for, e.g., developing a model for evaluating the level or degree of a disease or disorder of the tissue or organ. The invention thus provides methods for selecting response profiles according to the aspect or aspects of a tissue or organ for constructing a compendium. For example, to evaluate the general condition or status of a tissue or organ, i.e., to evaluate if the tissue or organ is normal or abnormal, a compendium may contain a large number of response profiles, each associated with one of a diverse collection of different conditions. In one embodiment, expression profiles associated with normal and a plurality of different types of abnormality in a tissue or organ are selected to construct a compendium. In a particular embodiment, expression profiles associated with normal and a plurality of different conditions of abnormal liver, such as necrosis, steatosis, DNA damage, cirrhosis, hypertrophy, phospholipidosis, and hepatic carcinoma, are selected to construct a liver status compendium. In another embodiment, to evaluate drug effect, e.g., toxicity, to liver, response profiles of liver cells of animals, e.g., rats, under different drugs are selected to construct a hepatotoxicity compendium. In a preferred embodiment, the hepatotoxicity compendium comprises a plurality of expression profiles representing the conditions of livers respectively under the treatments of at least 20, 50, 100, or 1,000 different hepatotoxins and/or dosages, and at least 5, 10, or 20 non-hepatotoxic compounds. The hepatotoxins preferably comprises of both chemicals and drugs and represent a plurality of different toxic mechanisms and lesions, e.g., necrosis, phospholipidosis, steatosis, cholestasis, and hypertrophy. In another preferred embodiment, the expression profiles in a hepatotoxicity compendium are measured after given period of treatment.

The compendium can also be constructed to cover different degrees or levels of a particular aspect of a tissue or organ, e.g., a particular type of abnormality or disease. In such cases, the compendium can comprise expression profiles associated with the tissue or organ having different degrees or levels of the type of abnormality or disease. In one embodiment, expression profiles associated with normal and a plurality of different levels of a particular abnormality in liver, e.g., different levels of necrosis, steatosis, DNA damage, cirrhosis, hypertrophy, phospholipidosis, or hepatic carcinoma, are used to construct a compendium. In another embodiment, to evaluate drug induced liver damage of a particular type, e.g., drug induced necrosis, phospholipidosis, steatosis, cholestasis, or hypertrophy, response profiles of liver cells of animals under different doses of one or more drugs which cause the type of liver damage are used to construct a hepatotoxicity compendium. In a preferred embodiment, the hepatotoxicity compendium comprises expression profiles each associated with a liver condition as a result of treatment of one of at least 10, 20, 50, or 100 different doses of a hepatotoxin. In another preferred embodiment, the expression profiles in a hepatotoxicity compendium are measured at a given time after the treatment.

In some embodiments, the profiles in a compendium can be divided into groups, each contains response profiles under some common values or ranges of values for a set of one or more biological variables. As one example, the profiles in a compendium can be divided into groups according to one or more clinical measures or a composite clinical score. In a preferred embodiment, each group contains response profiles each having a composite clinical score within a give range. As another example, the profiles in a compendium can be divided into groups based on the value of one or more variables characterizing the condition of the animal, e.g., identity and dosage of a drug used to treat the animal, the degree or level of a disease, etc.

In general, the more diverse the compendium with respect to the different perturbations, diseases, or disorders, etc., as the case may be, the more preferred is the compendium. For example, with respect to a compendium to be used to determine drug toxicity, it is preferred to use a compendium with different drugs such that toxicities via a diverse spectrum of different mechanisms of action, and/or different types of pathology, are represented. A model trained using such a compendium can be used to evaluate such diverse types of drug toxicities. By way of further example, a compendium of response profiles generated by use of drugs that only cause toxicity type A will be best for determining or predicting toxicity of compounds that cause toxicity type A. Furthermore, it is preferred that at least some response profiles be measured after a period of drug exposure, which period may, for example, involve repeated drug administration, but which period is shorter than the period of exposure needed for manifestation of tissue or organ damage as a result of the drug toxicity.

Such a compendium can be used for identifying cellular constituents markers and to construct models which can then be used to classify tissues or organs based on similarity of their cellular constituent profiles with a group of profiles of particular attributes.

As noted, in Section 5.1.2 above, the biological response to a perturbation m can be represented as the vector v^{(m)} whose individual elements vᵢ^{(m)} are the amplitude of the response of each cellular constituent i to the perturbation m (e.g., the logarithm of the ratio of the abundance or activity of cellular constituent i when the cell is subject to perturbation m to when the cell is not subject to perturbation m). Accordingly, the perturbation response profiles in a compendium of the present invention are most preferably obtained or measured under identical or at least substantially identical conditions that differ only by the particular perturbation of the response profile. In other words, the unperturbed or reference state of each perturbation response profile in the compendium is preferably identical for all of the perturbation response profiles. Likewise, the perturbed state of each perturbation response profile should differ from the unperturbed state by the specific perturbation of the perturbation response profile (e.g., the specific genetic mutation, the specific disease, the specific drug exposure, or the specific change in nutrient or other growth conditions).

For example, the perturbation response profiles are most preferably obtained for identical cell types. More specifically, the cells are preferably isogenic cells, or at least substantially isogenic cells, that are obtained from the same species of organism, and more preferably from the same tissue or same tissue type of that species of organism. The perturbation response profiles are also preferably obtained or measured from cells that are at the same stage of growth (i.e., cells that are in the same phase of the cell cycle). In embodiments in which the cells are cells from a multicellular organism such as a plant or an animal, the cells are preferably obtained from one or more individual organisms during the same developmental stage (e.g., cells from an embryonic organism or, alternatively, from an adult organism).

Further, the methods of the present invention can also employ a plurality of compendia, rather than only a single compendium, of perturbation response profiles. For example, it is possible to generate a plurality of “parallel” compendia encompassing a plurality of different conditions. Each of the compendia would then comprise perturbations response profiles for the same perturbations but under different baseline or unperturbed conditions. For example, the “parallel” compendia might encompass different nutrient conditions, different disease states, different stages of cell growth, different cell types (e.g., cells from different tissues of the same species of organism) or corresponding to different stages of development.

The cellular constituents in a profile in a compendium of the present invention can be organized or ordered according to “co-varying sets” (see, e.g., U.S. Pat. No. 6,203,987). Further, the response profiles of the compendium can also be ordered or “clustered” according to methods such as the methods described in U.S. Pat. No. 6,203,987 and PCT publication WO 00/35336.

In one embodiment of the invention, the overall expression patterns of the compendium can be determined using a suitable pattern recognition method, e.g., a two-dimensional cluster analysis (see, e.g., PCT publication WO 00/24936). Such two-dimensional clustering techniques can be used for confirming that the expression profiles show identifiable patterns correlating to the different conditions. Such two-dimensional clustering techniques can also be used for identifying sets of genes and experiments of particular interest. For example, the two-dimensional clustering techniques of this invention may be used to identify genes whose expression levels change significantly across the response profiles of different conditions in the compendium, e.g., genes whose expression levels change significantly across the response profiles in different sub-compendiums. The two-dimensional clustering techniques of this invention may also be used, e.g., to identify sets of cellular constituents and/or experiments that are associated with a particular biological pathway of interest. In one preferred embodiment of the invention, such sets of cellular constituents and/or experiments are used to determine consensus profiles for a particular biological response of interest. In other embodiments, identification of such sets of cellular constituents and/or experiments provide more precise indications of groupings cellular constituents, such as identification of genes involved in a particular biological pathway or response of interest.

Any clustering method can be used in the invention (see, e.g., U.S. Pat. No. 6,203,987 and PCT publication WO 00/35336). In one embodiment, a similarity between two profiles x(r) and x(s) is defined as

$$S = 1 - [ {\sum }_{i = 1}^{N} \frac{( {x}_{i} ( r ) - \overline{x} ( r ) )}{{σ}_{{x}_{i}} ( r )} \cdot \frac{( {x}_{i} ( s ) - \overline{x} ( s ) )}{{σ}_{{x}_{i}} ( s )} / \sqrt{{\sum }_{i = 1}^{N} {( \frac{{x}_{i} ( r ) - \overline{x} ( r )}{{σ}_{{x}_{i}} ( r )} )}^{2} \cdot {\sum }_{i = 1}^{N} {( \frac{( {x}_{i} ( s ) - \overline{x} ( s ) )}{{σ}_{{x}_{i}} ( s )} )}^{2}} ] \qquad ( 10 )$$

where x(r) and x(s) are two profiles with components of log ratio xᵢ(r) and xᵢ(s), σₓᵢ(r) and σₓᵢ(s) are the estimated errors associated with every measured ratios xᵢ(r) and xᵢ(s), respectively, and where i=1, . . . , N, N is the number of measurements in the profiles, e.g., transcriptional profiles, and where

$$\overline{x} ( j ) = {\sum }_{i = 1}^{N} \frac{{x}_{i} ( j )}{{σ}_{{x}_{i}}^{2} ( j )} / {\sum }_{i = 1}^{N} \frac{1}{{σ}_{{x}_{i}}^{2} ( j )} \qquad ( 11 )$$

where j is r or s, is the error-weighted arithmetic mean. To emphasize the importance of co-regulation in clustering rather than the amplitude of regulations, the correlation is utilized as a similarity metric. In another embodiment, the set of N cellular constituents are also clustered based on the similarities of their profiles from overall treatments in the compendium. The same similarity metric is used to define the distance, except that for each cellular constituent, e.g., each gene, the log ratios across all the treated samples were used to calculate the similarity metric.

In preferred embodiments, the measurements of cellular constituents in the response profiles are analyzed to identify significantly regulated cellular constituents, e.g., genes, in the compendium under a set of one or more biological variables. In one embodiment, cellular constituents whose measurements change more than a predetermined folds with p value smaller than a predetermined threshold in at least a predetermined number of profiles are identified as significantly regulated cellular constituents in the compendium. In one embodiment, cellular constituents whose measurements change more than 2, 3, 4 or 10 folds with p value <0.01, 0.001, or 0.0001 in at least 3, 5 or 10 profiles are identified as significantly regulated cellular constituents in the compendium. In preferred embodiments, cellular constituents which are not identified as significantly regulated in the compendium are discarded so as to reduce the number of measurements in each profile.

In a preferred embodiment, the statistical significance of the response of a gene in one or more profiles is also determined. In one embodiment, the measured response of a gene is transformed by a transformation as in Weng, U.S. patent application Ser. No. 10/349,364, filed on Jan. 22, 2003 and Weng, U.S. patent application Ser. No. 10/354,664, filed on Jan. 30, 2003, each of which is incorporated by reference herein in its entirety. The statistical significance of the response is then determined based on the transformed response. In one embodiment, the statistical significance is characterized by a p value, indicating the probability that the variation in the transformed response is due to random errors. In a preferred embodiment, genes whose responses have a fold change above a given threshold level with a p value less than a given threshold level are selected as significantly regulated genes.

In another embodiment, the statistical significance of the response of a gene is characterized by a percentile ranking (see, e.g., U.S. Pat. No. 6,351,712, which is incorporated herein by reference in its entirety). In one embodiment, if a gene of interest is present in the top 1% of up or down regulations in a profile, the percentile rank of the gene in the profile is expressed as a p value=0.01. The percentile rank of a gene in k profiles is given by

$$p = {\prod }_{i}^{k} {p}_{i} ,$$

where pᵢ is the p value of the gene in the ith profile. In one embodiment, those genes whose p value in one or more profiles is less than a threshold are identified. In a preferred embodiment, genes whose p value is less than 0.01 in at least 3 response profiles are identified as significantly regulated genes.

In another embodiment, a combination of an improved ANOVA method (Hughes et al., 2000, Cell 102: 109-26; Dai et al., 2002, Nucleic Acids Res 30: e86; Weng, U.S. patent application Ser. No. 10/349,364, filed on Jan. 22, 2003; Weng, U.S. patent application Ser. No. 10/354,664, filed on Jan. 30, 2003, each of which is incorporated by reference herein in its entirety) and fold changes are used to identify the significantly regulated cellular constituents. In a preferred embodiment, the clustering analysis is carried out with only the subset of significantly regulated cellular constituents.

It is further noted that the invention also contemplates “dynamic” databases or compendia of perturbation response profiles. In particular, the compendia of the invention can be continuously updated as additional modifications and perturbation experiments are performed so that the new perturbation response profiles are added to the database. In some embodiments of the dynamic database, the perturbation data and clinical measure data are stored in a series of relational tables in digital computer storage media (e.g., on one or more hard drives, CD-ROMs, floppy disks or DAT tapes to name a few). Preferably, the database is implemented in distributed system environments with client/server implementation, allowing multiuser and remote access. Access control and usage accounting are implemented in some embodiments of the database system. Relational database management systems and client/server environments are well documented in the art (see, for example, Nath, 1995, The Guide to SQL Server, 2nd Ed., Addison-Wesley Publishing Co.).

In a specific embodiment, the invention provides a rat liver compendium. The rat liver compendium is built with a set of compounds comprising the 59 compound listed in Table I. An rat liver toxicology oligonucleotide microarray containing approximately 25,000 probes were employed to build the compendium. In one embodiment, 267 global transcriptional profiles are included in the current compendium. In one embodiment, all profiles used to build the compendium come from rats receiving a 3-day treatment. Among the 59 compounds listed in Table I, 49 of them are known as liver toxicants. In one embodiment, twenty liver toxicants are administrated with both low range dose and high range dose, and twenty toxicants are administrated only with high range dose, whereas for the 10 compounds which are not previously observed or reported as having liver toxicity, both a low dose and high dose range have been employed. The detailed list of compounds and associated dose are summarized in Table I.

In one embodiment, utilizing a combination of an improved ANOVA method (Hughes et al., 2000, Cell 102: 109-26; Dai et al., 2002, Nucleic Acids Res 30: e86; Weng, U.S. patent application Ser. No. 10/349,364, filed on Jan. 22, 2003; Weng, U.S. patent application Ser. No. 10/354,664, filed on Jan. 30, 2003, each of which is incorporated by reference herein in its entirety) and fold changes. In the specific embodiment of liver toxicity, 2536 genes or reporting ESTs that change more than 3 folds with p value <0.01 in at least 3 profiles are identified as significantly regulated genes in the compendium. Two dimensional clustering using the similarity metric as described by Equations (10) and (11) are carried out with N=2,536. The set of 2,536 significantly regulated genes are also clustered based on the similarities of their profiles from overall treatments in the compendium.

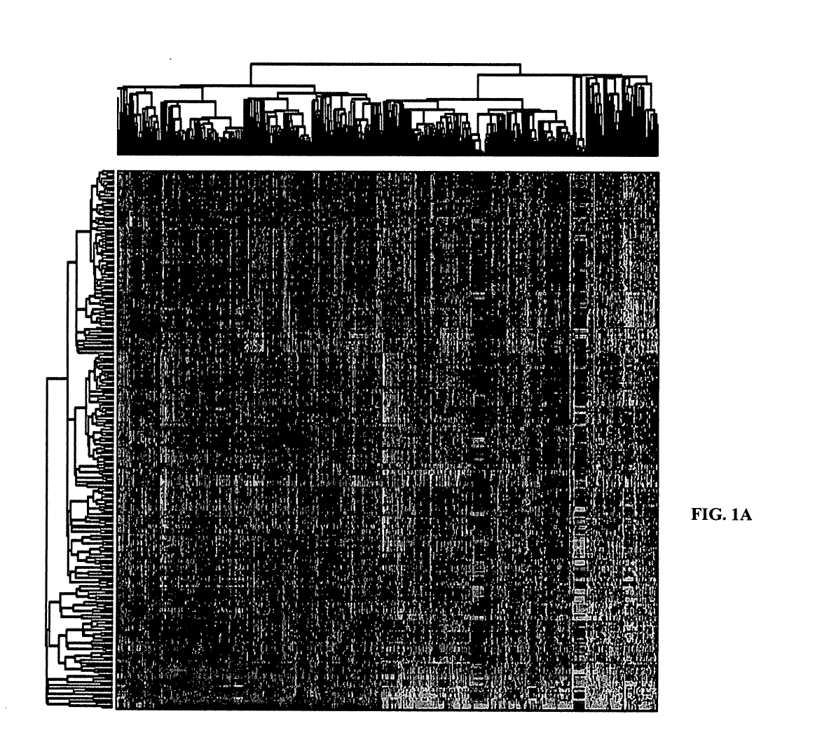

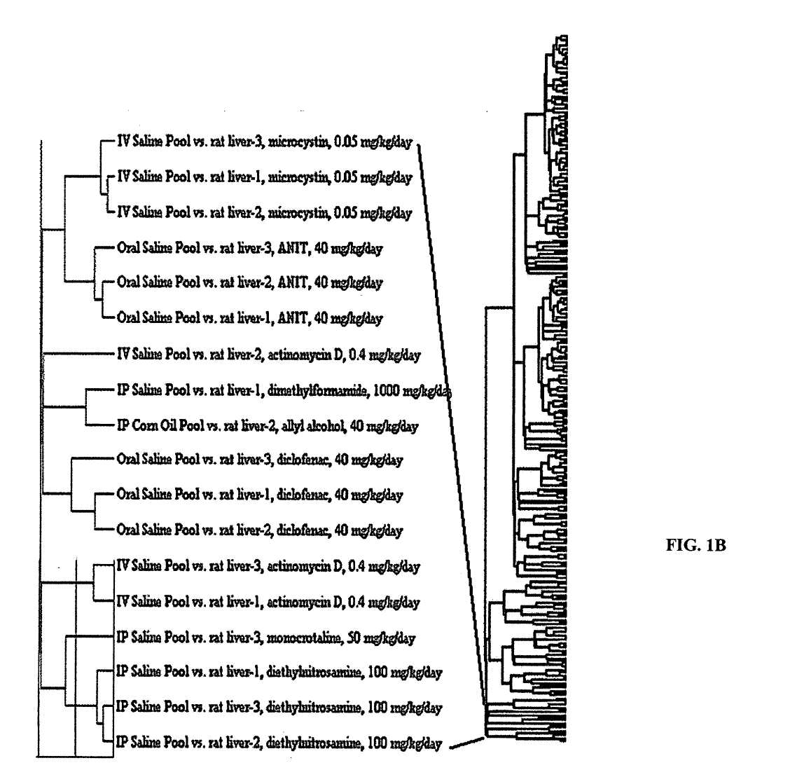

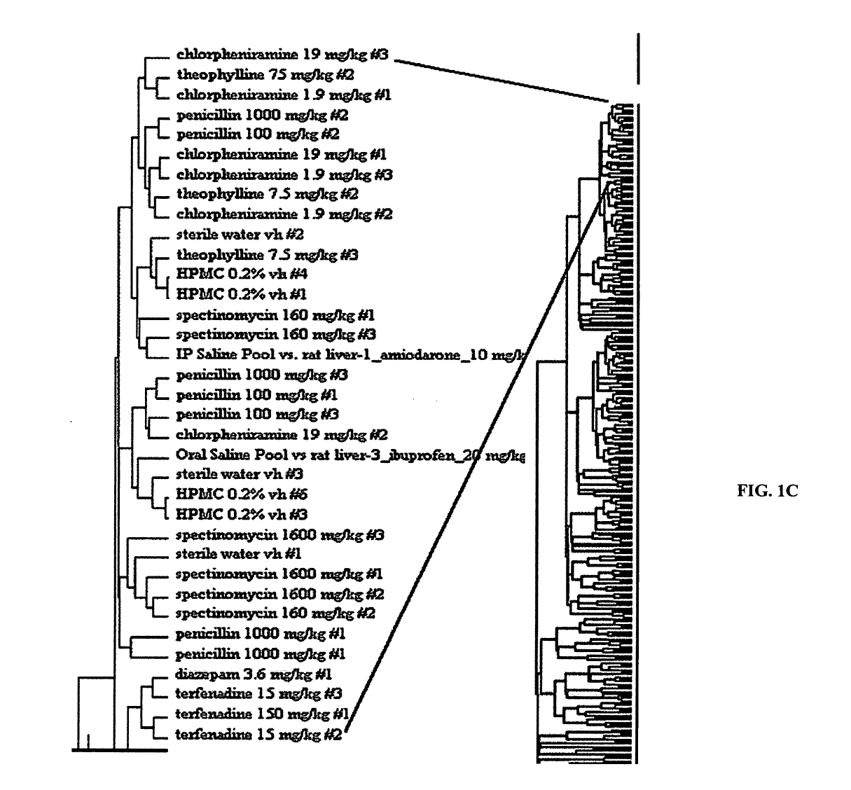

The unsupervised 2-dimensional hierarchical clustering demonstrates specific patterns among toxicants and non-hepatotoxins (FIG. 1A). In particular, distinctive expression patterns can be observed between non-hepatotoxins and toxicants. Genes highly regulated by toxicants did not overlap with genes regulated by non-hepatotoxins. Similarity clustering over the compound profile dimension further indicates the big distance between clusters of toxicants and clusters of non-hepatotoxins (FIG. 1A, B). On the other hand, a consistent expression pattern is observed within profiles from rat repeats that received treatments from the same compounds, toxicants or non-hepatotoxins (FIG. 1B, 1C). The observations suggest that transcriptional profiles in our rat compendium contain information for compound hepatotoxicity. The highly reproducible gene expression patterns can be used for compound hepatotoxicity prediction.

In some embodiments, cellular constituents, e.g., genes, which are significantly regulated by one or more common biological variables are selected as marker cellular constituents. In one embodiment, an ANOVA analysis is carried out to different groups of response profiles in the compendium, e.g., different groups based on the value of one or more variables characterizing the condition of the animal, e.g., identity and dosage of a drug used to treat the animal, the degree or level of a disease, etc. In a preferred embodiment, the marker cellular constituents are selected using the training data set. In another preferred embodiment, the selected cellular constituents are validated using the validating data set. Preferably, error-weighted measurements are used in to determine the marker cellular constituents. In one embodiment, the error-weighted measurements are error-weighted log ratios according to Eq. (12)

$${Xdev}_{i} = \frac{log {x}_{i}}{{σ}_{log {x}_{i}}} \qquad ( 12 )$$

where Xdevᵢ is the error, i.e., σ_{log x}_{ᵢ}, weighted log ratio of the ith measurement, xᵢ; i=1, 2, . . . , N; and N is the number of measurements in the profile. In a preferred embodiment, the marker genes are selected with an ANOVA p-value of a least <0.001, 0.0001, 0.000001, or 0.00000001. A skilled person in the art will be able to determine the desirable p-value based on, e.g., the number of profiles used, the biological variables the marker genes are intended to represent, and so on.

In a specific embodiment of liver damage compendium with 267 profiles, genes significantly regulated in the hepatotoxic treated group are selected as marker genes. In one embodiment, 238 genes are selected by one way ANOVA between the hepatotoxic compounds treated and non-hepatotoxic compounds treated liver in the training data set using error-weighted log ratios as described by Eq. 12 with a p-value <0.0000001. The 238 marker genes are listed in Table II. The 238 genes are therefore genes whose changes are associated with liver toxicity, but not confounding with specific pharmacological effects. The invention therefore also provides a method for determining cellular response of a liver to a compound by measuring the expression levels of the 238 markers genes.

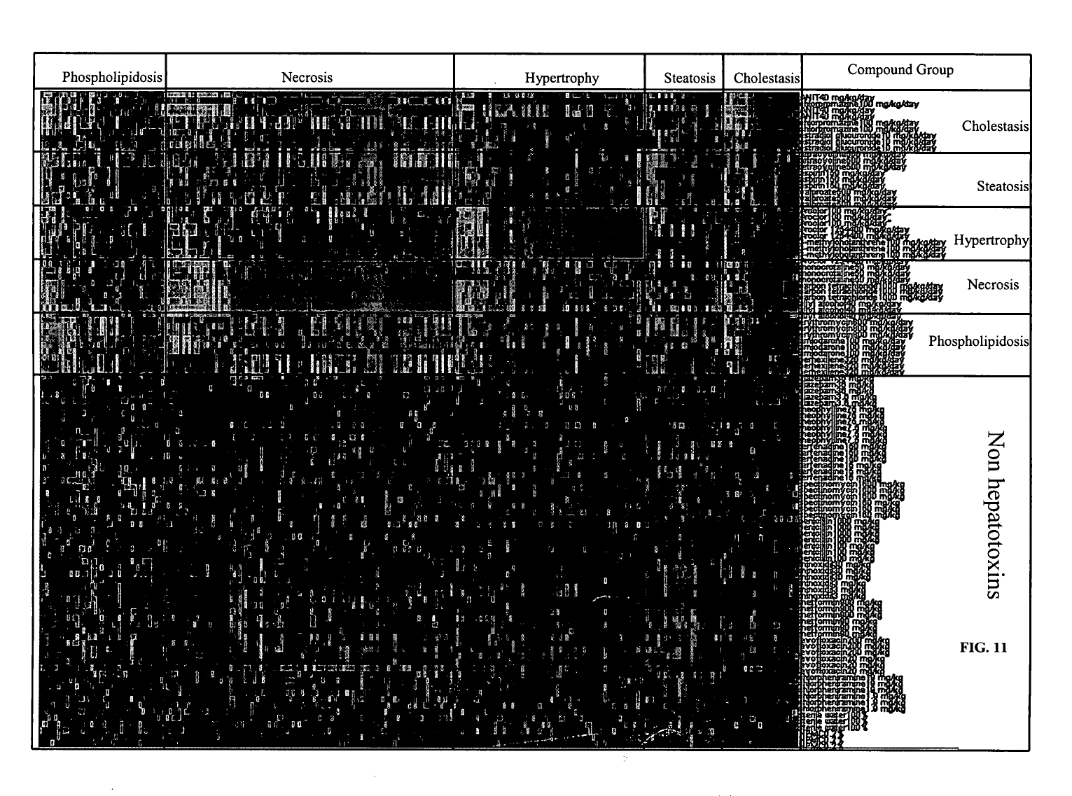

In another embodiment, expression pattern corresponding to necrosis, steatosis, cholestasis, hypertrophy and phospholipidosis, respectively, are also detected. A example of such expression patterns are shown in FIG. 11.

### 5.4.2. Methods of Predicting Tissue Condition

The response profiles in a suitable compendium or compendia, e.g., as described in Section 5.4.1, can be used to establish a model estimator, e.g., a neural network model, for predicting a status or condition or a tissue or organ using its response profile. Preferably, the response profiles comprise only meausrements of cellular constituent markers, e.g., cellular constituent markers selected by the method as described in Section 5.4.1. The response profiles can each comprise measurements of at least 20, 50, 100, 150, 200, 250, 300, 400, or 500 cellular constituent markers. The model estimator can then be used to predict the status of a tissue from its cellular constituent profile.

In one embodiment, a subset of the response profiles in the compendium is selected as training data set. All or a portion of the remaining response profiles in the compendium can be used as a validating data set for testing the prediction power of the model. In a preferred embodiment, the response profiles in the training set are randomly selected. Preferably, the training set consists of at least 20, 50, 80, 90, or 95 percent of the response profiles in the compendium. In the specific embodiment of liver damage compendium consisting of 267 transcriptional profiles, 212 (80% of 267) profiles with their associated clinical chemistry measurements are randomly selected into the training data set and the remaining 54 (20% of 267) profiles with their associated clinical chemistry measurements are used as a validating data set. An exemplary procedure for ab initio prediction of hepatotoxicity based on transcriptional profiles is illustrated in FIG. 2.

In some embodiments of the invention, the variable dimension of cellular constituents is reduced, for example, when the variable dimension in a profile is larger than a desired number. The desired variable dimension depends in part on the number of profiles in the training data set. Preferably the variable dimension is such that the model obtained from the profiles in the training data set is not over-fitted. In preferred embodiments, the desired variable dimension is about 50%, 20%, 10%, or 5% of the number of profiles in the training data set. Various methods known in the art can be used to reduce the variable dimension. Preferably, the profiles with reduced variable dimension retains main regulation information across treatment groups for each individual cellular constituent.

The reduction of variable dimension can be achieved by transforming the measurement data in a profile using a suitable data transformation. The transformation transforms a profile which is in a pattern space of high dimensionality to a feature space of an appropriate reduced dimensionality. In one embodiment, a profile of measurements, e.g., the Xdev's, for significantly regulated cellular constituents is transformed by a suitable transformation into a “profile of features” such that the main regulation information across treatment groups for each individual cellular constituent is retained, but the variable dimension is reduced. In preferred embodiments, the transformation is selected such that the variable dimension is reduced to about 50%, 20%, 10%, or 5% of the number of profiles in the training data set. The “profile of features”, i.e., the data in the feature space, is used to classify the original profile comprising measurements of cellular constituents.

In a preferred embodiment, a wavelet transformation, e.g., with Daubechies wavelet function at a suitable level, is used to transform the profiles. The wavelet transformation is widely applied in image processing industry and other procedure. A detail description of the transformation procedure can be found in Matlab Wavelet tool box user's guide. A description of the mathematical formulas can be found in the book “Introduction to Wavelets” (Charles K. Chui 1992, Academic Press, Inc.). Methods of using wavelet transformation to reduce the dimension of a pattern space to a feature space is described in U.S. Pat. No. 5,867,118, which is incorporated herein by reference in its entirety. Each profile is treated as a pattern in a pattern space and the coefficients between the ratios and a suitable level of a set of wavelet functions, e.g., Daubechies wavelet functions, are calculated. The coefficients, which indicates how similar the trend of the ratio from individual profiles to the curve of the wavelet function, e.g., the Daubechies function as defined mathematically in the following Eqs14 and 15, are used to represent the regulation features in a profile. For each profile y, a set of coefficients P(y) are obtained by

$$P ( y ) = {\sum }_{l = 0}^{K - 1} {C}_{l}^{K - 1 + l} \cdot {y}^{l} \qquad ( 13 )$$

where Cᵢ^{K-1+l} is the binomial coefficient, K is the level of the transformation.
And

$$\begin{matrix}{\vert {m}_{0} ( ω ) \vert}^{2} = {( {cos}^{2} ( \frac{ω}{2} ) )}^{K} P ( {sin}^{2} ( \frac{ω}{2} ) ) \\ where & ( 14 ) \\ {m}_{0} ( ω ) = \frac{1}{\sqrt{2}} {\sum }_{l = 0}^{2 K - 1} {h}_{l} {ⅇ}^{- ⅈ l ω} & ( 15 )\end{matrix}$$

In a preferred embodiment, K is selected such that the variable dimension is reduced to 50%, 25%, 10%, or 5% of the original dimension. In a preferred embodiment, K is selected such that the variable dimension is reduced to no more than 50%, 20%, 10%, or 5% of the sample size, i.e., the number of profiles.

In another embodiment, the variable dimension is reduced using principal component analysis (see, e.g., Butte, 2002, Nat Rev Drug Discov 1: 951-60).

In the specific embodiment of liver damage compendium consisting of 267 transcriptional profiles. The ratios of all 238 marker gene in each profile is treated as data in time domain and the coefficients between the ratios and the 5^{th} approximation of the Wavelet Daubechies function describe curve are calculated. Daubechies Wavelets at level 5 is used to transform the 238 marker genes' profiles in both the training and validating data set to 31 transformed variables.

In other embodiments, the variable dimension can be reduced by selecting a subset of the significantly regulated genes. In one embodiment, the subset of genes are selected based on co-variation, e.g., selecting one or more than one genes from each co-varying geneset. Genesets can be determined according to any method known in the art (see, e.g., U.S. Pat. No. 6,203,987 and PCT publications WO99/58720 and WO 00/35336, each of which is incorporated herein by reference in its entirety).

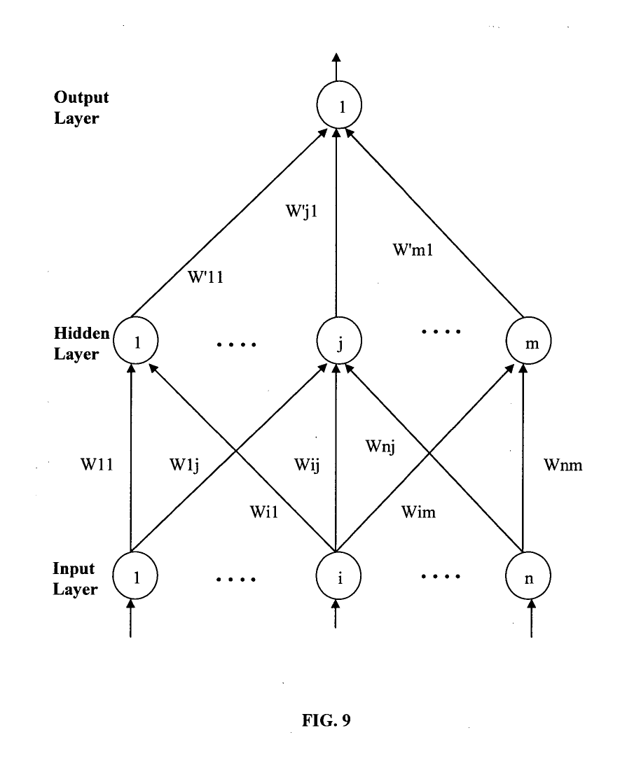

In one embodiment, a neural network estimator is trained for prediction of the condition of the tissue or organ from the response profile. Any suitable neural network architecture can be employed in the present invention. Preferably, the neural network is a multilayer neural network. FIG. 9 is an illustrative schematic of a multilayer neural network. A detailed description of the mathematical method and implementation method can be found in the Bishop's (1995) Neural networks for pattern recognition (Oxford, Clarendon) and Nabney's (2001) Netlab: Algorithms for pattern recognition (London, Springer). Preferably, a multi-layer perceptron (MLP) is employed as the architecture of the neural network. In a preferred embodiment, a three layer neural network with an input and an output layers and one hidden layer in between the input and the output layer is employed. The optimal number of nodes in the hidden layer is determined by cross-validation based on the training data set.

The neural network is trained using all or a portion of the data in the training data set or the transformed data, e.g., data characterizing features in a profile, and one or more associated biological or clinical variables, including composite clinical scores. In preferred embodiment, the transformed data are used to train the neural network to establish an estimator of a CCS for animal i
CCS(i)=f(z_{1,i}, z_{2,i}, . . . , z_{n,i}) (16)
where {Z_{k,i}} are the data in the training data set or the transformed data, k=1, . . . , n, n is the number of input variable.

The training of the neural network can be carried out by standard method known in the art. For example, in an embodiment employing a three layer neural network architecture of a two-layer feed-forward network and one hidden layer is first initialized using randomly selected weights for the hidden units (or nods). The weights for the hidden units are determined from the training data set via gradient search. Specifically, the derivative between the expected output, e.g., a given CCS value, and the calculated output associated with the set of weights for the hidden units is calculated. Then another set of weights is selected and the associated derivative is calculated. If the derivative from the latter set of weight is smaller than the former set, the latter set is retained. The iteration continues until the derivative is smaller than a predetermined threshed or until a preset number of iterations, e.g., 1,000 iterations, have been carried out. The set of weights and the associated derivative for the architecture are taken as the optimal structure. In one embodiment, the optimal number of hidden nods are determined by determining and comparing a plurality of neural network architectures each having a different number of hidden nods, and selecting the architecture with the smallest derivative. For example, the optimal structure of an architecture with 1 hidden nod is first determined. The architecture is then changed by increasing the hidden unit by 1 and the optimal structure for an architecture with 2 hidden units is determined. The process iterates until the number of hidden units equals the number of input variables. The architecture, as represented by the number of hidden units and the weights, with the smallest derivative is selected as the optimal architecture for the training data set. This architecture is retained as the trained model estimator for predicting the condition or status of a tissue or organ. The sensitivity and specificity of the model estimator can be evaluated by calculating the derivative of the optimal structure determined from the previous step with validating data set.

Preferably, the trained model estimator is examined by the independent validating data set. In one embodiment, prediction error is estimated by the average of the deviation between the expected value and the predicted value based on the profiles in the validating data set. In another embodiment, to determine whether the error associated with the trained model is significantly different from random error, Monte Carlo simulations are conducted and the estimated error from random distribution is determined. The number of Monte Carlo simulations sufficient to determine the error distribution can be readily determined by one skilled in the art. Often, at least 500 to about 5000 simulations are performed. The estimated error is then compared with the error associated with the model. If the estimated error is higher than the error associated with the model, e.g., with a p-value <0.05, the model is accepted.

In another preferred embodiment, the specificity and sensitivity of the trained model estimator are further evaluated using data in the validating data set to determined the rates of false positives and false negatives. In one embodiment, this is achieved by comparing the number of predicted positives detected by the predicted CCS's of the profiles in the validating data set and the number of actual positives as determined by actually measured clinical measures to determine the rates of false positives and false negatives. In one embodiment, the predicted positives are identified by comparing the positives detected by the predicted CCS's of the profiles in the validating data set with the pre-established CCS threshold.

In the specific embodiment of a liver damage compendium consisting of 267 transcriptional profiles, an optimal model neural network structure is determined by a cross-validating sampling approach, described by way of example as follows. A set of 212 expression profiles is selected randomly from total 267 profiles. The ratios of 238 marker genes in each profile among the 212 randomly selected profiles are transformed into 31 variables using the wavelet transformation described supra. The hepatotoxicity score and the 31 transformed variables are utilized as the training data set to estimate the relationship between the hepatotoxicity score and the transformed expression profiles. In particular, 80% of the 212 training data set profiles are randomly chosen and utilized as a training set and the rest of 20% of 212 profiles are used to determine the estimated error associated with certain neural network structure. A neural network structure with one hidden layer of 15 units is determined to have the lowest estimated error rate as estimated from the 20% profiles in the training data set.

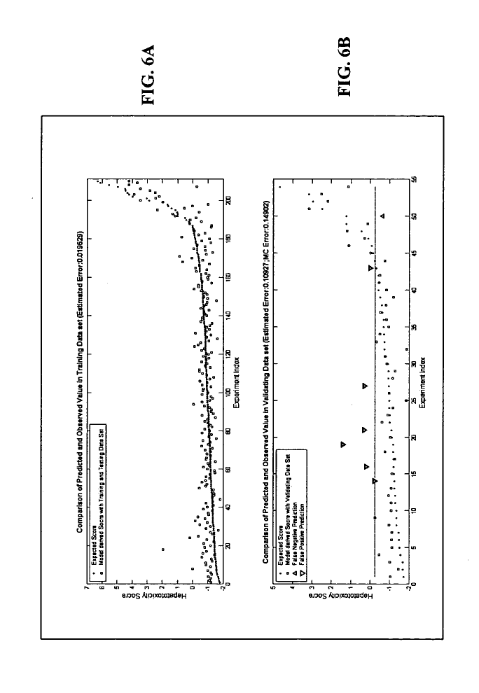

In the specific embodiment, the 31 transformed variables from the 238 reporter genes are utilized as independent variables, i.e., input variables for the neural network. The hepatotoxicity scores associated with individual profiles are used as the dependent variable, i.e., the outcome of the neural network. The multi-layer perceptron (MLP) is employed as the architecture of the neural network. An example of prediction from this trained neural network is illustrated (FIG. 6A). At the top panel, profiles are arranged according to their associated hepatotoxicity score, shown as the unfilled squares. The predicted hepatotoxicity scores from the trained model with the obtained model estimator are shown as circular dots. Prediction error is estimated by the average of the deviation between the expected value and the predicted value of the hepatotoxicity score for the 212 profiles. The estimated error for prediction from the training set is 0.08.

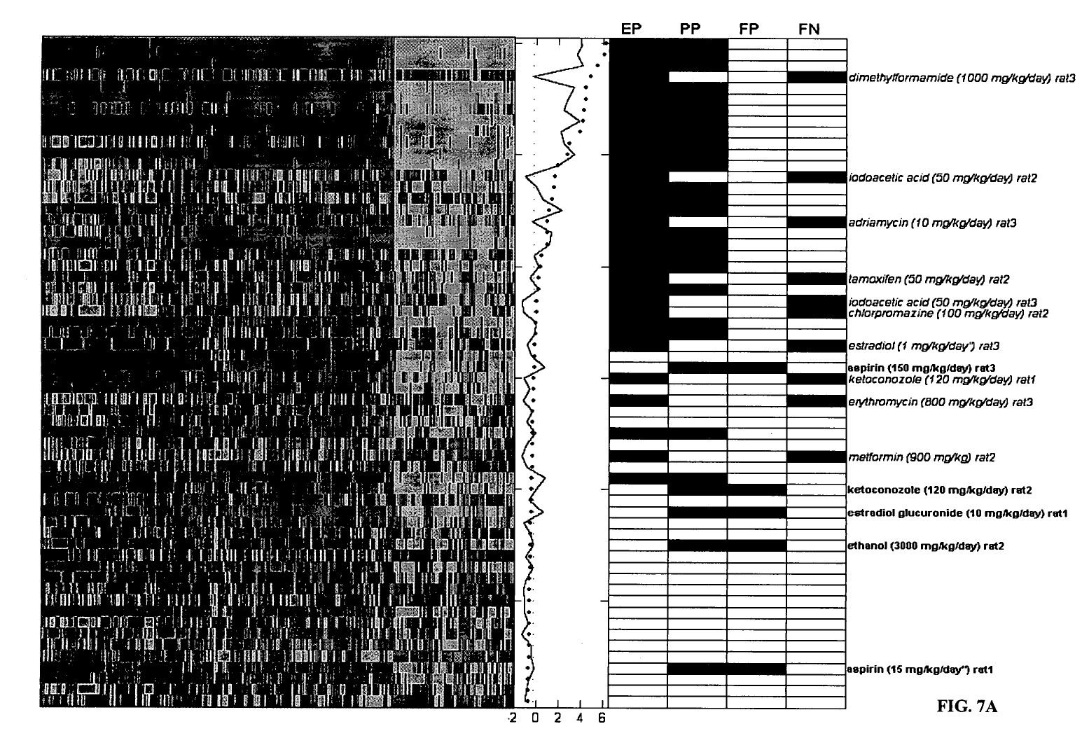

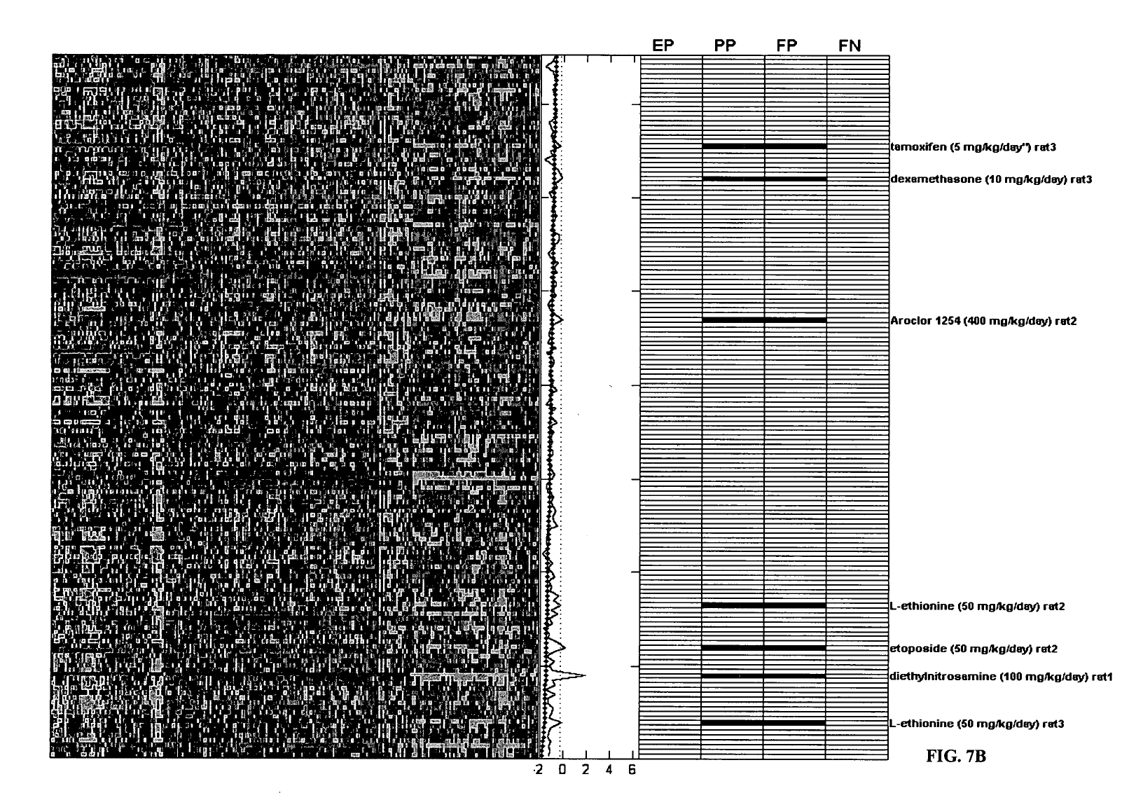

The specificity and sensitivity of the trained model in the training data set can be further examined with the pre-established liver hepatotoxicity score threshold (FIGS. 7A-B). Utilizing the positives (indicated as EP) detected by the combination of five clinical chemistry measurements as a substitute of the gold standard, we determined the number of false positives (FP) and false negatives (FN) by comparing the positives obtained from prediction (PP) with the EP. Among the 212 profiles, the model reports 12 false positives and 10 false negatives with 89.6% of prediction accuracy.

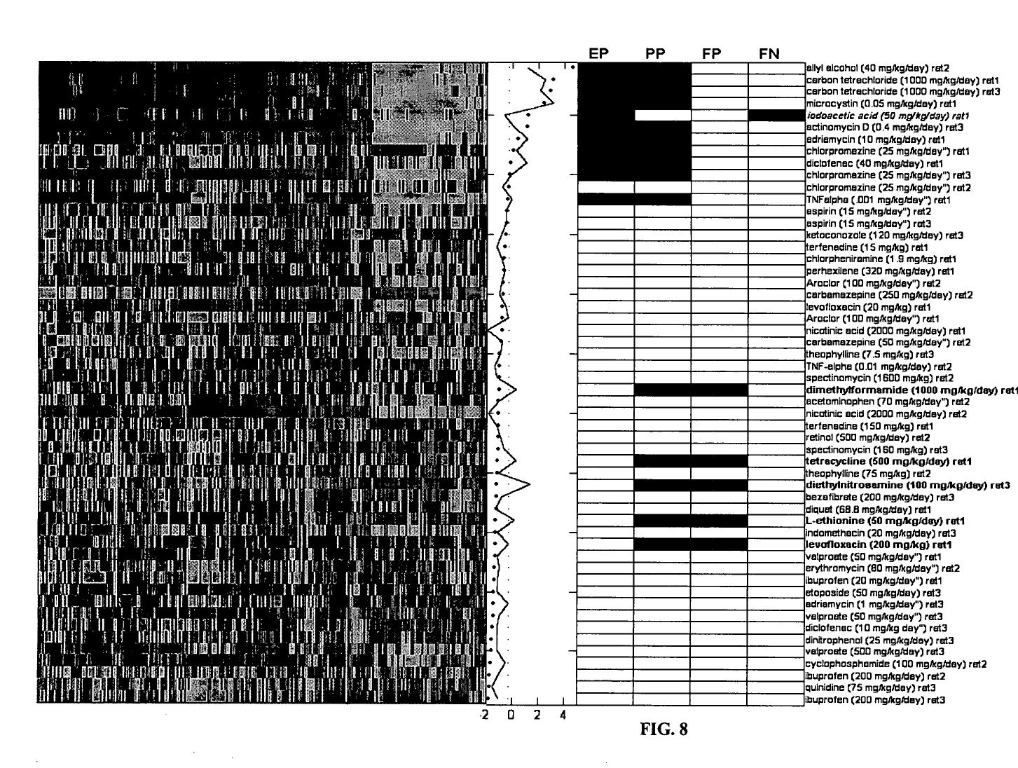

The accuracy and generality of the trained model can be examined by an independent data set with 54 expression profiles (FIG. 6B and FIG. 8). At the bottom panel of FIG. 6, profiles are arranged according to their associated hepatotoxicity scores, shown as square dots. The predicted hepatotoxicity scores were predicted from the previously trained model with data from the validating data set are shown as circular dots. Prediction error is estimated by the average of the deviation between the expected value and the predicted value based on the 54 profiles. The estimated error for prediction from the validating data set is 0.632.

To determine whether the error associated with the trained model is significantly different from random error, 5000 times of Monte Carlo simulation have been conducted and the estimated error from random distribution is 1.177, significantly higher from the error associated with the trained model (p value <0.05).

The specificity and sensitivity of the trained model in the validating data set can be confirmed with the pre-established liver hepatotoxicity score threshold (FIG. 8). Utilizing the positives (indicated as EP) detected by the combination of five clinical chemistry measurements of those 54 profiles as gold standard, the number of FP and the number of FN can be determined by comparing the positive obtained from PP with the EP. Among the 54 profiles, the model reported 5 false positives and 1 false negative with a 88% of prediction accuracy.

To further evaluate the accuracy of the model, the predictions of the model were compared with the pathological observations in the 5 false positives and 1 false negatives from the validating data set. The five false positives detected by our trained model include profiles from No.1 rat receiving dimethlformamide (1000 mg/kg/day), No.1 rat receiving tetracycline (500 mg/kg/day), No. 3 rat receiving diethylnitrosamine (100 mg/kg/day), No.1 rat receiving L-ethionine(50 mg/kg/day) and levofloxain (200 mg/kg/day). Although among them, levofloxacin is a non-hepatotoxin, noticeable pathological changes were discovered in the rest of the four compound-treated groups. Further optimization of reporter genes and transformation may help to eliminate the mistakenly classified levofloxacin and the false negative iodoacetic acid profile.

It will be apparent to one skilled in the art that other supervised machine learning algorithms, such as Bayesian network and supporting vector machine, can also be used to determine a model estimator of a clinical measure based on profile of cellular constituents, e.g., the association between the composite clinical score, e.g., the hepatotoxicity score, and the transcriptional profiles.

In another embodiment, the selection of marker genes is further optimized, e.g., iteratively with the model estimator and/or the associated transformation. Such optimization further improves the accuracy of the prediction model.

The invention provides method of determining the condition or status of a tissue or organ comprising determining a composite clinical score based on a profile of one or more cellular constituent markers using a model estimator. The model estimator can be determined by as described above. Preferably, the profile comprises more than 5, 10, 100 or 200 markers. In a preferred embodiment, the cellular constituent markers are gene markers. In one embodiment, the profile is measured using cells obtained from the tissue or organ of an animal. In anther embodiment, the profile is measured using in vitro cells of the tissue or organ.

The invention also provides a model estimator as described in this section for determining the condition or status or the tissue or organ based on a profile of one or more cellular constituent markers. The model estimator is preferably in the form of a computer program. The model estimator can be used to estimate a composite clinical score from the profile of one or more cellular constituent markers.

### 5.5. Implementation Systems and Methods

The analytical methods of the present invention can preferably be implemented using a computer system, such as the computer system described in this section, according to the following programs and methods. Such a computer system can also preferably store and manipulate measured signals obtained in various experiments that can be used by a computer system implemented with the analytical methods of this invention. Accordingly, such computer systems are also considered part of the present invention.

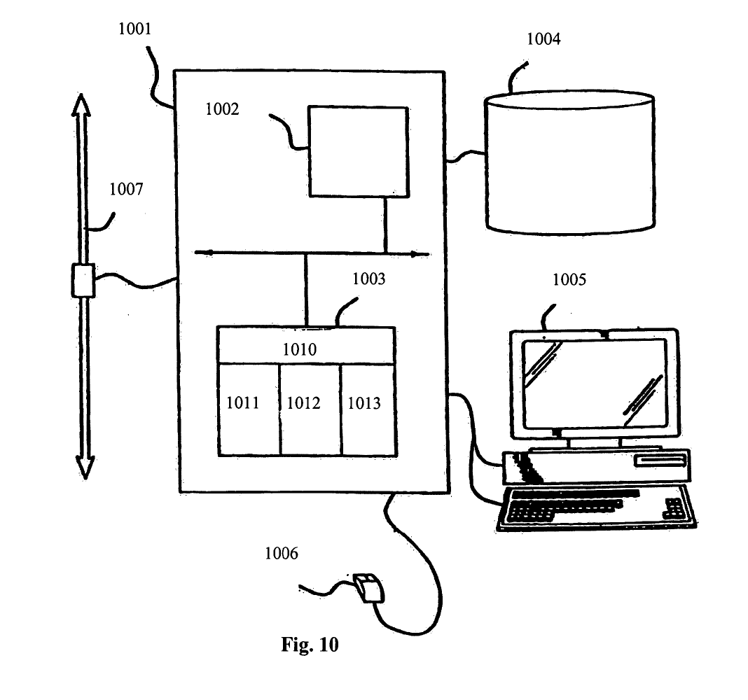

An exemplary computer system suitable from implementing the analytic methods of this invention is illustrated in FIG. 10. Computer system 1001 is illustrated here as comprising internal components and as being linked to external components. The internal components of this computer system include one or more processor elements 1002 interconnected with a main memory 1003. For example, computer system 1001 can be an Intel Pentium IV®-based processor of 2 GHZ or greater clock rate and with 256 MB or more main memory. In a preferred embodiment, computer system 1001 is a cluster of a plurality of computers comprising a head “node” and eight sibling “nodes,” with each node having a central processing unit (“CPU”). In addition, the cluster also comprises at least 128 MB of random access memory (“RAM”) on the head node and at least 256 MB of RAM on each of the eight sibling nodes. Therefore, the computer systems of the present invention are not limited to those consisting of a single memory unit or a single processor unit.

The external components can include a mass storage 1004. This mass storage can be one or more hard disks that are typically packaged together with the processor and memory. Such hard disk are typically of 10 GB or greater storage capacity and more preferably have at least 40 GB of storage capacity. For example, in a preferred embodiment, described above, wherein a computer system of the invention comprises several nodes, each node can have its own hard drive. The head node preferably has a hard drive with at least 10 GB of storage capacity whereas each sibling node preferably has a hard drive with at least 40 GB of storage capacity. A computer system of the invention can further comprise other mass storage units including, for example, one or more floppy drives, one more CD-ROM drives, one or more DVD drives or one or more DAT drives.

Other external components typically include a user interface device 1005, which is most typically a monitor and a keyboard together with a graphical input device 1006 such as a “mouse.” The computer system is also typically linked to a network link 1007 which can be, e.g., part of a local area network (“LAN”) to other, local computer systems and/or part of a wide area network (“WAN”), such as the Internet, that is connected to other, remote computer systems. For example, in the preferred embodiment, discussed above, wherein the computer system comprises a plurality of nodes, each node is preferably connected to a network, preferably an NFS network, so that the nodes of the computer system communicate with each other and, optionally, with other computer systems by means of the network and can thereby share data and processing tasks with one another.

Loaded into memory during operation of such a computer system are several software components that are also shown schematically in FIG. 10. The software components comprise both software components that are standard in the art and components that are special to the present invention. These software components are typically stored on mass storage such as the hard drive 1004, but can be stored on other computer readable media as well including, for example, one or more floppy disks, one or more CD-ROMs, one or more DVDs or one or more DATs. Software component 1010 represents an operating system which is responsible for managing the computer system and its network interconnections. The operating system can be, for example, of the Microsoft Windows™ family such as Windows 95, Window 98, Windows NT, Windows 2000 or Windows XP. Alternatively, the operating software can be a Macintosh operating system, a UNIX operating system or a LINUX operating system. Software components 1011 comprises common languages and functions that are preferably present in the system to assist programs implementing methods specific to the present invention. Languages that can be used to program the analytic methods of the invention include, for example, C and C++, FORTRAN, PERL, HTML, JAVA, and any of the UNIX or LINUX shell command languages such as C shell script language. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

Software component 1012 comprises any analytic methods of the present invention described supra, preferably programmed in a procedural language or symbolic package. For example, software component 1012 preferably includes programs that cause the processor to implement steps of accepting a plurality of measured signals and storing the measured signals in the memory. For example, the computer system can accept measured signals that are manually entered by a user (e.g., by means of the user interface). More preferably, however, the programs cause the computer system to retrieve measured signals from a database. Such a database can be stored on a mass storage (e.g., a hard drive) or other computer readable medium and loaded into the memory of the computer, or the compendium can be accessed by the computer system by means of the network 1007.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

### 5.6. Methods for Determining Biological State and Biological Response

In the present invention, cellular constituent profiles can comprise measurements of a plurality of cellular constituents in a sample of a tissue or organ or responses of a cell sample of a tissue or organ to a perturbation. The cellular constituent profiles can be measured from cell samples subject to different conditions, e.g., under different perturbations. The cell sample can be from any tissue or organ from any organism, e.g., eukaryote, mammal, primate, human, non-human animal such as a dog, cat, horse, cow, mouse, rat, Drosophila, C. elegans, etc., plant such as rice, wheat, bean, tobacco, etc., and fungi. The cell sample can be from a diseased or healthy tissue or organ of an organism, or an organism predisposed to disease. The cell sample can be of a particular tissue type or development stage or subjected to a particular perturbation (stimulus). This section and its subsections provides some exemplary methods for obtaining cellular constituent profiles of cell samples. One of skill in the art would appreciate that this invention is not limited to the following specific methods for measuring the expression profiles and responses of a biological system.

### 5.6.1. Transcript Assays Using Microarrays

In the methods of the invention, the expression state or the transcriptional state of a tissue or organ may be determined by monitoring expression profiles. For example, polynucleotide probe arrays may be used for simultaneous determination of the expression levels of a plurality of genes and methods for designing and making such polynucleotide probe arrays.

The expression level of a nucleotide sequence in a gene can be measured by any high throughput techniques. However measured, the result is either the absolute or relative amounts of transcripts or response data, including but not limited to values representing abundance ratios.

Preferably, measurement of the expression profile is made by hybridization to transcript arrays, which are described in this subsection.

In a preferred embodiment, the present invention makes use of “transcript arrays” or “profiling arrays”. Transcript arrays can be employed for analyzing the expression profile in a cell sample and especially for measuring the expression profile of a cell sample of a particular tissue type or developmental state or exposed to a drug of interest or to perturbations to a biological pathway of interest. In another embodiment, the cell sample can be from a patient, e.g., a diseased cell sample, and preferably can be compared to a healthy cell sample.

In one embodiment, an expression profile is obtained by hybridizing detectably labeled polynucleotides representing the nucleotide sequences in mRNA transcripts present in a cell (e.g., fluorescently labeled cDNA synthesized from total cell mRNA) to a microarray. A microarray is an array of positionally-addressable binding (e.g., hybridization) sites on a support for representing many of the nucleotide sequences in the genome of a cell or organism, preferably most or almost all of the genes. Each of such binding sites consists of polynucleotide probes bound to the predetermined region on the support. Microarrays can be made in a number of ways, of which several are described herein below. However produced, microarrays share certain characteristics. The arrays are reproducible, allowing multiple copies of a given array to be produced and easily compared with each other. Preferably, the microarrays are made from materials that are stable under binding (e.g., nucleic acid hybridization) conditions. The microarrays are preferably small, e.g., between about 1 cm² and 25 cm², preferably about 1 to 3 cm². However, both larger and smaller arrays are also contemplated and may be preferable, e.g., for simultaneously evaluating a very large number of different probes.

Preferably, a given binding site or unique set of binding sites in the microarray will specifically bind (e.g., hybridize) to a nucleotide sequence in a single gene from a cell or organism (e.g., to exon of a specific mRNA or a specific cDNA derived therefrom).

The microarrays used in the methods and compositions of the present invention include one or more test probes, each of which has a polynucleotide sequence that is complementary to a subsequence of RNA or DNA to be detected. Each probe preferably has a different nucleic acid sequence, and the position of each probe on the solid surface of the array is preferably known. Indeed, the microarrays are preferably addressable arrays, more preferably positionally addressable arrays. More specifically, each probe of the array is preferably located at a known, predetermined position on the solid support such that the identity (i.e., the sequence) of each probe can be determined from its position on the array (i.e., on the support or surface). In some embodiments of the invention, the arrays are ordered arrays.

Preferably, the density of probes on a microarray or a set of microarrays is about 100 different (i.e., non-identical) probes per 1 cm² or higher. More preferably, a microarray used in the methods of the invention will have at least 550 probes per 1 cm², at least 1,000 probes per 1 cm², at least 1,500 probes per 1 cm² or at least 2,000 probes per 1 cm². In a particularly preferred embodiment, the microarray is a high density array, preferably having a density of at least about 2,500 different probes per 1 cm². The microarrays used in the invention therefore preferably contain at least 2,500, at least 5,000, at least 10,000, at least 15,000, at least 20,000, at least 25,000, at least 50,000 or at least 55,000 different (i.e., non-identical) probes.

In one embodiment, the microarray is an array (i.e., a matrix) in which each position represents a discrete binding site for a nucleotide sequence of a transcript encoded by a gene (e.g., for an exon of an mRNA or a cDNA derived therefrom). The collection of binding sites on a microarray contains sets of binding sites for a plurality of genes. For example, in various embodiments, the microarrays of the invention can comprise binding sites for products encoded by fewer than 50% of the genes in the genome of an organism. Alternatively, the microarrays of the invention can have binding sites for the products encoded by at least 50%, at least 75%, at least 85%, at least 90%, at least 95%, at least 99% or 100% of the genes in the genome of an organism. In other embodiments, the microarrays of the invention can having binding sites for products encoded by fewer than 50%, by at least 50%, by at least 75%, by at least 85%, by at least 90%, by at least 95%, by at least 99% or by 100% of the genes expressed by a cell of an organism. The binding site can be a DNA or DNA analog to which a particular RNA can specifically hybridize. The DNA or DNA analog can be, e.g., a synthetic oligomer or a gene fragment, e.g. corresponding to an exon.

In some embodiments of the present invention, a gene or an exon in a gene is represented in the profiling arrays by a set of binding sites comprising probes with different polynucleotides that are complementary to different coding sequence segments of the gene or an exon of the gene. Such polynucleotides are preferably of the length of 15 to 200 bases, more preferably of the length of 20 to 100 bases, most preferably 40-60 bases. It will be understood that each probe sequence may also comprise linker sequences in addition to the sequence that is complementary to its target sequence. As used herein, a linker sequence refers to a sequence between the sequence that is complementary to its target sequence and the surface of support. For example, in preferred embodiments the profiling arrays of the invention comprise one probe specific to each target gene or exon. However, if desired, the profiling arrays may contain at least 2, 5, 10, 100, 1000 probes specific to some target genes or exons. For example, the array may contain probes tiled across the sequence of the longest mRNA isoform of a gene at single base steps.

It will be appreciated that when cDNA complementary to the RNA of a cell is made and hybridized to a microarray under suitable hybridization conditions, the level of hybridization to the site in the array corresponding to an exon of any particular gene will reflect the prevalence in the cell of mRNA or mRNAs containing the exon transcribed from that gene. For example, when detectably labeled (e.g., with a fluorophore) cDNA complementary to the total cellular mRNA is hybridized to a microarray, the site on the array corresponding to an exon of a gene (i.e., capable of specifically binding the product or products of the gene expressing) that is not transcribed or is removed during RNA splicing in the cell will have little or no signal (e.g., fluorescent signal), and an exon of a gene for which the encoded mRNA expressing the exon is prevalent will have a relatively strong signal. The relative abundance of different mRNAs produced from the same gene by alternative splicing is then determined by the signal strength pattern across the whole set of exons monitored for the gene.

In preferred embodiments, cDNAs from cell samples from two different conditions are hybridized to the binding sites of the microarray using a two-color protocol. In the case of drug responses one cell sample is exposed to a drug and another cell sample of the same type is not exposed to the drug. In the case of pathway responses one cell is exposed to a pathway perturbation and another cell of the same type is not exposed to the pathway perturbation. The cDNA derived from each of the two cell types are differently labeled (e.g., with Cy3 and Cy5) so that they can be distinguished. In one embodiment, for example, cDNA from a cell treated with a drug (or exposed to a pathway perturbation) is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular exon detected.

In the example described above, the cDNA from the drug-treated (or pathway perturbed) cell will fluoresce green when the fluorophore is stimulated and the cDNA from the untreated cell will fluoresce red. As a result, when the drug treatment has no effect, either directly or indirectly, on the transcription and/or post-transcriptional splicing of a particular gene in a cell, the exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the drug-exposed cell is treated with a drug that, directly or indirectly, change the transcription and/or post-transcriptional splicing of a particular gene in the cell, the exon expression pattern as represented by ratio of green to red fluorescence for each exon binding site will change. When the drug increases the prevalence of an mRNA, the ratios for each exon expressed in the mRNA will increase, whereas when the drug decreases the prevalence of an mRNA, the ratio for each exons expressed in the mRNA will decrease.

The use of a two-color fluorescence labeling and detection scheme to define alterations in gene expression has been described in connection with detection of mRNAs, e.g., in Shena et al., 1995, Quantitative monitoring of gene expression patterns with a complementary DNA microarray, Science 270: 467-470, which is incorporated by reference in its entirety for all purposes. The scheme is equally applicable to labeling and detection of exons. An advantage of using cDNA labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular exon in, e.g., a drug-treated or pathway-perturbed cell and an untreated cell. Furthermore, labeling with more than two colors is also contemplated in the present invention. In some embodiments of the invention, at least 5, 10, 20, or 100 dyes of different colors can be used for labeling. Such labeling permits simultaneous hybridizing of the distinguishably labeled cDNA populations to the same array, and thus measuring, and optionally comparing the expression levels of, mRNA molecules derived from more than two samples. Dyes that can be used include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41, cyamine dyes, including but are not limited to Cy3, Cy3.5 and Cy5; BODIPY dyes including but are not limited to BODIPY-FL, BODIPY-TR, BODIPY-TMR, BODIPY-630/650, and BODIPY-650/670; and ALEXA dyes, including but are not limited to ALEXA-488, ALEXA-532, ALEXA-546, ALEXA-568, and ALEXA-594; as well as other fluorescent dyes which will be known to those who are skilled in the art.

In some embodiments of the invention, hybridization data are measured at a plurality of different hybridization times so that the evolution of hybridization levels to equilibrium can be determined. In such embodiments, hybridization levels are most preferably measured at hybridization times spanning the range from 0 to in excess of what is required for sampling of the bound polynucleotides (i.e., the probe or probes) by the labeled polynucleotides so that the mixture is close to or substantially reached equilibrium, and duplexes are at concentrations dependent on affinity and abundance rather than diffusion. However, the hybridization times are preferably short enough that irreversible binding interactions between the labeled polynucleotide and the probes and/or the surface do not occur, or are at least limited. For example, in embodiments wherein polynucleotide arrays are used to probe a complex mixture of fragmented polynucleotides, typical hybridization times may be approximately 0-72 hours. Appropriate hybridization times for other embodiments will depend on the particular polynucleotide sequences and probes used, and may be determined by those skilled in the art (see, e.g., Sambrook et al., Eds., 1989, Molecular Cloning: A Laboratory Manual, 2nd ed., Vol. 1-3, Cold Spring Harbor Laboratory, Cold Spring Harbor, N.Y.).

In one embodiment, hybridization levels at different hybridization times are measured separately on different, identical microarrays. For each such measurement, at hybridization time when hybridization level is measured, the microarray is washed briefly, preferably in room temperature in an aqueous solution of high to moderate salt concentration (e.g., 0.5 to 3 M salt concentration) under conditions which retain all bound or hybridized polynucleotides while removing all unbound polynucleotides. The detectable label on the remaining, hybridized polynucleotide molecules on each probe is then measured by a method which is appropriate to the particular labeling method used. The resulted hybridization levels are then combined to form a hybridization curve. In another embodiment, hybridization levels are measured in real time using a single microarray. In this embodiment, the microarray is allowed to hybridize to the sample without interruption and the microarray is interrogated at each hybridization time in a non-invasive manner. In still another embodiment, one can use one array, hybridize for a short time, wash and measure the hybridization level, put back to the same sample, hybridize for another period of time, wash and measure again to get the hybridization time curve.

Preferably, at least two hybridization levels at two different hybridization times are measured, a first one at a hybridization time that is close to the time scale of cross-hybridization equilibrium and a second one measured at a hybridization time that is longer than the first one. The time scale of cross-hybridization equilibrium depends, inter alia, on sample composition and probe sequence and may be determined by one skilled in the art. In preferred embodiments, the first hybridization level is measured at between 1 to 10 hours, whereas the second hybridization time is measured at about 2, 4, 6, 10, 12, 16, 18, 48 or 72 times as long as the first hybridization time.

### 5.6.2. Preparing Probes for Microarrays

As noted above, the “probe” to which a particular polynucleotide molecule, such an exon, specifically hybridizes according to the invention is a complementary polynucleotide sequence. Preferably one or more probes are selected for each target exon. For example, when a minimum number of probes are to be used for the detection of an exon, the probes normally comprise nucleotide sequences greater than about 40 bases in length. Alternatively, when a large set of redundant probes is to be used for an exon, the probes normally comprise nucleotide sequences of about 40-60 bases. The probes can also comprise sequences complementary to full length exons. The lengths of exons can range from less than 50 bases to more than 200 bases. Therefore, when a probe length longer than exon is to be used, it is preferable to augment the exon sequence with adjacent constitutively spliced exon sequences such that the probe sequence is complementary to the continuous mRNA fragment that contains the target exon. This will allow comparable hybridization stringency among the probes of an exon profiling array. It will be understood that each probe sequence may also comprise linker sequences in addition to the sequence that is complementary to its target sequence.

The probes may comprise DNA or DNA “mimics” (e.g., derivatives and analogues) corresponding to a portion of each exon of each gene in an organism's genome. In one embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (P CR) amplification of exon segments from genomic DNA, cDNA (e.g., by RT-PCR), or cloned sequences. PCR primers are preferably chosen based on known sequence of the exons or cDNA that result in amplification of unique fragments (i.e., fragments that do not share more than 10 bases of contiguous identical sequence with any other fragment on the microarray). Computer programs that are well known in the art are useful in the design of primers with the required specificity and optimal amplification properties, such as Oligo version 5.0 (National Biosciences). Typically each probe on the microarray will be between 20 bases and 600 bases, and usually between 30 and 200 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., 1986, Nucleic Acid Res. 14: 5399-5407; McBride et al., 1983, Tetrahedron Lett. 24: 246-248). Synthetic sequences are typically between about 15 and about 600 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363: 566-568; U.S. Pat. No. 5,539,083).

In alternative embodiments, the hybridization sites (i.e., the probes) are made from plasmid or phage clones of genes, cDNAs (e.g., expressed sequence tags), or inserts therefrom (Nguyen et al., 1995, Genomics 29: 207-209).

### 5.6.3. Attaching Probes to the Solid Surface

Preformed polynucleotide probes can be deposited on a support to form the array. Alternatively, polynucleotide probes can be synthesized directly on the support to form the array. The probes are attached to a solid support or surface, which may be made, e.g., from glass, plastic (e.g., polypropylene, nylon), polyacrylamide, nitrocellulose, gel, or other porous or nonporous material.

A preferred method for attaching the nucleic acids to a surface is by printing on glass plates, as is described generally by Schena et al, 1995, Science 270: 467-470. This method is especially useful for preparing microarrays of cDNA (See also, DeRisi et al, 1996, Nature Genetics 14: 457-460; Shalon et al., 1996, Genome Res. 6: 639-645; and Schena et al., 1995, Proc. Natl. Acad. Sci. U.S.A. 93: 10539-11286).

A second preferred method for making microarrays is by making high-density polynucleotide arrays. Techniques are known for producing arrays containing thousands of oligonucleotides complementary to defined sequences, at defined locations on a surface using photolithographic techniques for synthesis in situ (see, Fodor et al., 1991, Science 251: 767-773; Pease et al., 1994, Proc. Natl. Acad. Sci. U.S.A. 91: 5022-5026; Lockhart et al., 1996, Nature Biotechnology 14: 1675; U.S. Pat. Nos. 5,578,832; 5,556,752; and 5,510,270) or other methods for rapid synthesis and deposition of defined oligonucleotides (Blanchard et al., Biosensors & Bioelectronics 11: 687-690). When these methods are used, oligonucleotides (e.g., 60-mers) of known sequence are synthesized directly on a surface such as a derivatized glass slide. The array produced can be redundant, with several polynucleotide molecules per exon.

Other methods for making microarrays, e.g., by masking (Maskos and Southern, 1992, Nucl. Acids. Res. 20: 1679-1684), may also be used. In principle, and as noted supra, any type of array, for example, dot blots on a nylon hybridization membrane (see Sambrook et al., supra) could be used. However, as will be recognized by those skilled in the art, very small arrays will frequently be preferred because hybridization volumes will be smaller.

In a particularly preferred embodiment, microarrays of the invention are manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioelectronics 11: 687-690; Blanchard, 1998, in Synthetic DNA Arrays in Genetic Engineering, Vol. 20, J. K. Setlow, Ed., Plenum Press, New York at pages 111-123; and U.S. Pat. No. 6,028,189 to Blanchard. Specifically, the polynucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Polynucleotide probes are normally attached to the surface covalently at the 3′ end of the polynucleotide. Alternatively, polynucleotide probes can be attached to the surface covalently at the 5′ end of the polynucleotide (see for example, Blanchard, 1998, in Synthetic DNA Arrays in Genetic Engineering, Vol. 20, J. K. Setlow, Ed., Plenum Press, New York at pages 111-123).

### 5.6.4. Target Polynucleotide Molecules

Target polynucleotides which may be analyzed by the methods and compositions of the invention include RNA molecules such as, but by no means limited to messenger RNA (mRNA) molecules, ribosomal RNA (rRNA) molecules, cRNA molecules (i.e., RNA molecules prepared from cDNA molecules that are transcribed in vivo) and fragments thereof. Target polynucleotides which may also be analyzed by the methods and compositions of the present invention include, but are not limited to DNA molecules such as genomic DNA molecules, cDNA molecules, and fragments thereof including oligonucleotides, ESTs, STSs, etc.

The target polynucleotides may be from any source. For example, the target polynucleotide molecules may be naturally occurring nucleic acid molecules such as genomic or extragenomic DNA molecules isolated from an organism, or RNA molecules, such as mRNA molecules, isolated from an organism. Alternatively, the polynucleotide molecules may be synthesized, including, e.g., nucleic acid molecules synthesized enzymatically in vivo or in vitro, such as cDNA molecules, or polynucleotide molecules synthesized by PCR, RNA molecules synthesized by in vitro transcription, etc. The sample of target polynucleotides can comprise, e.g., molecules of DNA, RNA, or copolymers of DNA and RNA. In preferred embodiments, the target polynucleotides of the invention will correspond to particular genes or to particular gene transcripts (e.g., to particular mRNA sequences expressed in cells or to particular cDNA sequences derived from such mRNA sequences). However, in many embodiments, particularly those embodiments wherein the polynucleotide molecules are derived from mammalian cells, the target polynucleotides may correspond to particular fragments of a gene transcript. For example, the target polynucleotides may correspond to different exons of the same gene, e.g., so that different splice variants of that gene may be detected and/or analyzed.

In preferred embodiments, the target polynucleotides to be analyzed are prepared in vitro from nucleic acids extracted from cells. For example, in one embodiment, RNA is extracted from cells (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation and an oligo dT purification (Chirgwin et al., 1979, Biochemistry 18: 5294-5299). In another embodiment, RNA is extracted from cells using guanidinium thiocyanate lysis followed by purification on RNeasy columns (Qiagen). cDNA is then synthesized from the purified mRNA using, e.g., oligo-dT or random primers. In preferred embodiments, the target polynucleotides are cRNA prepared from purified messenger RNA extracted from cells. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997; see also, U.S. Pat. No. 6,271,002 and PCT publication WO 02/44399. Both oligo-dT primers (U.S. Pat. Nos. 5,545,522 and 6,132,997) or random primers (U.S. Provisional Patent Application Ser. No. 60/253,641, filed on Nov. 28, 2000, by Ziman et al.) that contain an RNA polymerase promoter or complement thereof can be used. Preferably, the target polynucleotides are short and/or fragmented polynucleotide molecules which are representative of the original nucleic acid population of the cell.

The target polynucleotides to be analyzed by the methods and compositions of the invention are preferably detectably labeled. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled.

Preferably, the detectable label is a fluorescent label, e.g., by incorporation of nucleotide analogs. Other labels suitable for use in the present invention include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Preferred radioactive isotopes include ³²P, ³⁵S, ¹⁴C, ¹⁵N and ¹²⁵I. Fluorescent molecules suitable for the present invention include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41. Fluroescent molecules that are suitable for the invention further include: cyamine dyes, including by not limited to Cy3, Cy3.5 and Cy5; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold. Alternatively, in less preferred embodiments the target polynucleotides may be labeled by specifically complexing a first group to the polynucleotide. A second group, covalently linked to an indicator molecules and which has an affinity for the first group, can be used to indirectly detect the target polynucleotide. In such an embodiment, compounds suitable for use as a first group include, but are not limited to, biotin and iminobiotin. Compounds suitable for use as a second group include, but are not limited to, avidin and streptavidin.

### 5.6.5. Hybridization to Microarrays

As described supra, nucleic acid hybridization and wash conditions are chosen so that the polynucleotide molecules to be analyzed by the invention (referred to herein as the “target polynucleotide molecules) specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93: 10614). Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press, San Diego, Calif.

Particularly preferred hybridization conditions for use with the screening and/or signaling chips of the present invention include hybridization at a temperature at or near the mean melting temperature of the probes (e.g., within 5° C., more preferably within 2° C.) in 1 M NaCl, 50 mM MES buffer (pH 6.5), 0.5% sodium Sarcosine and 30% formamide.

### 5.6.6. Signal Detection and Data Analysis

It will be appreciated that when target sequences, e.g., cDNA or cRNA, complementary to the RNA of a cell is made and hybridized to a microarray under suitable hybridization conditions, the level of hybridization to the site in the array corresponding to an exon of any particular gene will reflect the prevalence in the cell of mRNA or mRNAs containing the exon transcribed from that gene. For example, when detectably labeled (e.g., with a fluorophore) cDNA complementary to the total cellular mRNA is hybridized to a microarray, the site on the array corresponding to an exon of a gene (i.e., capable of specifically binding the product or products of the gene expressing) that is not transcribed or is removed during RNA splicing in the cell will have little or no signal (e.g., fluorescent signal), and an exon of a gene for which the encoded mRNA expressing the exon is prevalent will have a relatively strong signal. The relative abundance of different mRNAs produced by from the same gene by alternative splicing is then determined by the signal strength pattern across the whole set of exons monitored for the gene.

In preferred embodiments, target sequences, e.g., cDNAs or cRNAs, from two different cells are hybridized to the binding sites of the microarray. In the case of drug responses one cell sample is exposed to a drug and another cell sample of the same type is not exposed to the drug. In the case of pathway responses one cell is exposed to a pathway perturbation and another cell of the same type is not exposed to the pathway perturbation. The cDNA or cRNA derived from each of the two cell types are differently labeled so that they can be distinguished. In one embodiment, for example, cDNA from a cell treated with a drug (or exposed to a pathway perturbation) is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular exon detected.

In the example described above, the cDNA from the drug-treated (or pathway perturbed) cell will fluoresce green when the fluorophore is stimulated and the cDNA from the untreated cell will fluoresce red. As a result, when the drug treatment has no effect, either directly or indirectly, on the transcription and/or post-transcriptional splicing of a particular gene in a cell, the exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the drug-exposed cell is treated with a drug that, directly or indirectly, changes the transcription and/or post-transcriptional splicing of a particular gene in the cell, the exon expression pattern as represented by ratio of green to red fluorescence for each exon binding site will change. When the drug increases the prevalence of an mRNA, the ratios for each exon expressed in the mRNA will increase, whereas when the drug decreases the prevalence of an mRNA, the ratio for each exons expressed in the mRNA will decrease.

The use of a two-color fluorescence labeling and detection scheme to define alterations in gene expression has been described in connection with detection of mRNAs, e.g., in Shena et al., 1995, Quantitative monitoring of gene expression patterns with a complementary DNA microarray, Science 270: 467-470, which is incorporated by reference in its entirety for all purposes. The scheme is equally applicable to labeling and detection of exons. An advantage of using target sequences, e.g., cDNAs or cRNAs, labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular exon in, e.g., a drug-treated or pathway-perturbed cell and an untreated cell.

In other preferred embodiments, single-channel detection methods, e.g., using one-color fluorescence labeling, are used (see U.S. provisional patent application Ser. No. 60/227,966, filed on Aug. 25, 2000). In this embodiment, arrays comprising reverse-complement (RC) probes are designed and produced. Because a reverse complement of a DNA sequence has sequence complexity that is equivalent to the corresponding forward-strand (FS) probe that is complementary to a target sequence with respect to a variety of measures (e.g., measures such as GC content and GC trend are invariant under the reverse complement), a RC probe is used to as a control probe for determination of level of non-specific cross hybridization to the corresponding FS probe. The significance of the FS probe intensity of a target sequence is determined by comparing the raw intensity measurement for the FS probe and the corresponding raw intensity measurement for the RC probe in conjunction with the respective measurement errors. In a preferred embodiment, an exon is called present if the intensity difference between the FS probe and the corresponding RC probe is significant. More preferably, an exon is called present if the FS probe intensity is also significantly above background level. Single-channel detection methods can be used in conjunction with multi-color labeling. In one embodiment, a plurality of different samples, each labeled with a different color, is hybridized to an array. Differences between FS and RC probes for each color are used to determine the level of hybridization of the corresponding sample.

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Res. 6: 639-645). In a preferred embodiment, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6: 639-645. Alternatively, the fiber-optic bundle described by Ferguson et al., 1996, Nature Biotech. 14: 1681-1684, may be used to monitor mRNA abundance levels at a large number of sites simultaneously.

Signals are recorded and, in a preferred embodiment, analyzed by computer, e.g., using a 12 bit analog to digital board. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event.

According to the method of the invention, the relative abundance of an mRNA and/or an exon expressed in an mRNA in two cells or cell lines is scored as perturbed (i.e., the abundance is different in the two sources of mRNA tested) or as not perturbed (i.e., the relative abundance is the same). As used herein, a difference between the two sources of RNA of at least a factor of about 25% (i.e., RNA is 25% more abundant in one source than in the other source), more usually about 50%, even more often by a factor of about 2 (i.e., twice as abundant), 3 (three times as abundant), or 5 (five times as abundant) is scored as a perturbation. Present detection methods allow reliable detection of difference of an order of about 3-fold to about 5-fold, but more sensitive methods are expected to be developed.

It is, however, also advantageous to determine the magnitude of the relative difference in abundances for an mRNA and/or an exon expressed in an mRNA in two cells or in two cell lines. This can be carried out, as noted above, by calculating the ratio of the emission of the two fluorophores used for differential labeling, or by analogous methods that will be readily apparent to those of skill in the art.

### 5.6.7. Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93: 659-663). Other methods statistically sample cDNA pools, such as by sequencing sufficient bases (e.g., 20-50 bases) in each of multiple cDNAs to identify each cDNA, or by sequencing short tags (e.g., 9-10 bases) that are generated at known positions relative to a defined mRNA end (see, e.g., Velculescu, 1995, Science 270: 484-487).

### 5.7. Measurement of Other Aspects of the Biological State

In various embodiments of the present invention, aspects of the biological state other than the transcriptional state, such as the translational state, the activity state, or mixed aspects can be measured to produce the measured signals to be analyzed according to the invention. Thus, in such embodiments, gene expression data may include translational state measurements or even protein expression measurements. In fact, in some embodiments, rather than using gene expression interaction maps based on gene expression, protein expression interaction maps based on protein expression maps are used. Details of embodiments in which aspects of the biological state other than the transcriptional state are described in this section.

### 5.7.1. Embodiments Based on Translational State Measurements

Measurement of the translational state may be performed according to several methods. For example, whole genome monitoring of protein (i.e., the “proteome,” Goffeau et al., 1996, Science 274: 546-567; Aebersold et al., 1999, Nature Biotechnology 10: 994-999) can be carried out by constructing a microarray in which binding sites comprise immobilized, preferably monoclonal, antibodies specific to a plurality of protein species encoded by the cell genome (see, e.g., Zhu et al., 2001, Science 293: 2101-2105; MacBeath et al., 2000, Science 289: 1760-63; de Wildt et al., 2000, Nature Biotechnology 18: 989-994). Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated and measured by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Natl. Acad. Sci. USA 93: 1440-1445; Sagliocco et al., 1996, Yeast 12: 1519-1533; Lander, 1996, Science 274: 536-539; and Beaumont et al., Life Science News 7, 2001, Amersham Pharmacia Biotech. The resulting electropherograms can be analyzed by numerous techniques, including mass spectrometric techniques, Western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

### 5.7.2. Embodiments Based on Other Aspects of the Biological State

Even though methods of this invention are illustrated by embodiments involving gene expression, the methods of the invention are applicable to any cellular constituent that can be monitored. In particular, where activities of proteins can be measured, embodiments of this invention can use such measurements. Activity measurements can be performed by any functional, biochemical, or physical means appropriate to the particular activity being characterized. Where the activity involves a chemical transformation, the cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

### 5.8. Measurement of Drug Response Data

Drug responses are obtained for use in the instant invention by measuring the gene expression state changed by drug exposure. The biological response described on the exon level can also be measured by exon profiling methods. The measured response data include values representing gene expression level values or gene expression level ratios for a plurality of genes.

To measure drug response data, cell can be exposed to graded levels of the drug or drug candidate of interest. When the cells are grown in vitro, the compound is usually added to their nutrient medium. The drug is added in a graded amount that depends on the particular characteristics of the drug, but usually will be between about 1 ng/ml and 100 mg/ml. In some cases a drug will be solubilized in a solvent such as DMSO.

The exon expression profiles of cells exposed to the drug and of cells not exposed to the drug are measured according to the methods described in the previous section. Preferably, gene transcript arrays are used to find the genes with altered gene expression profiles due to exposure to the drug.

It is preferable for measurements of drug responses, in the case of two-colored differential hybridization described above, to measure with reversed labeling. Also, it is preferable that the levels of drug exposure used provide sufficient resolution of rapidly changing regions of the drug response, e.g., by using approximately ten levels of drug exposure.

### 5.9. Methods for Probing Biological States

One aspect of the invention provides methods for the analysis of biological state. The methods of this invention are also useful for the analysis of responses of a cell sample to perturbations designed to probe cellular state. Preferred perturbations are those that cause a change in the amount of alternative splicing that occurs in one or more RNA transcripts. This section provides some illustrative methods for probing gene expression states and protein abundances and acitivities. See PCT publication WO 00/24936 for more detailed descriptions of these method.

Methods for targeted perturbation of cells are widely known and applied in the art. For example, such methods include use of titratable expression systems, use of transfection or viral transduction systems, direct modifications to RNA abundances or activities, direct modifications of protein abundances, direct modification of protein activities including use of drugs (or chemical moieties in general), and post-transcriptional gene silencing (PTGS) or RNA interference (RNAi).

In mammalian cells, several means of titrating expression of genes are available (Spencer, 1996, Trends Genet. 12: 181-187). For example, the Tet system is widely used, both in its original form, the “forward” system, in which addition of doxycycline represses transcription, and in the newer “reverse” system, in which doxycycline addition stimulates transcription (Gossen et al., 1995, Proc. Natl. Acad. Sci. USA 89: 5547-5551; Hoffmann et al., 1997, Nucl. Acids. Res. 25: 1078-1079; Hofmann et al., 1996, Proc. Natl. Acad. Sci. USA 83: 5185-5190; Paulus et al., 1996, Journal of Virology 70: 62-67). Another commonly used controllable promoter system in mammalian cells is the ecdysone-inducible system developed by Evans and colleagues (No et al., 1996, Proc. Nat. Acad. Sci. USA 93: 3346-3351), where expression is controlled by the level of muristerone added to the cultured cells. Finally, expression can be modulated using the “chemical-induced dimerization” (CID) system developed by Schreiber, Crabtree, and colleagues (Belshaw et al., 1996, Proc. Nat. Acad. Sci. USA 93: 4604-4607; Spencer, 1996, Trends Genet. 12: 181-187) and similar systems in yeast. In this system, the gene of interest is put under the control of the CID-responsive promoter, and transfected into cells expressing two different hybrid proteins, one comprised of a DNA-binding domain fused to FKBP12, which binds FK506. The other hybrid protein contains a transcriptional activation domain also fused to FKBP12. The CID inducing molecule is FK1012, a homodimeric version of FK506 that is able to bind simultaneously both the DNA binding and transcriptional activating hybrid proteins. In the graded presence of FK1012, graded transcription of the controlled gene is activated.

Transfection or viral transduction of target genes can introduce controllable perturbations in biological gene expression states in mammalian cells. Preferably, transfection or transduction of a target gene can be used with cells that do not naturally express the target gene of interest. Such non-expressing cells can be derived from a tissue not normally expressing the target gene or the target gene can be specifically mutated in the cell. The target gene of interest can be cloned into one of many mammalian expression plasmids, for example, the pcDNA3.1+/− system (Invitrogen, Inc.) or retroviral vectors, and introduced into the non-expressing host cells. Transfected or transduced cells expressing the target gene may be isolated by selection for a drug resistance marker encoded by the expression vector. The level of gene transcription is monotonically related to the transfection dosage. In this way, the effects of varying levels of the target gene may be investigated. Other methods of modifying RNA abundances and activities and thus gene abundances include ribozymes, antisense species, and RNA aptamers (Good et al., 1997, Gene Therapy 4: 45-54). Controllable application or exposure of a cell to these entities permits controllable perturbation of RNA abundances.

Ribozymes are RNAs which are capable of catalyzing RNA cleavage reactions. (Cech, 1987, Science 236: 1532-1539; PCT International Publication WO 90/11364, published Oct. 4, 1990; Sarver et al., 1990, Science 247: 1222-1225). “Hairpin” and “hammerhead” RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA. (Haseloff et al., 1988, Nature 334: 585-591; Koizumi et al., 1988, FEBS Lett., 228: 228-230; Koizumi et al., 1988, FEBS Lett., 239: 285-288). Ribozyme methods involve exposing a cell to, inducing expression in a cell, etc. of such small RNA ribozyme molecules. (Grassi and Marini, 1996, Annals of Medicine 28: 499-510; Gibson, 1996, Cancer and Metastasis Reviews 15: 287-299).

In another embodiment, activity of a target RNA (preferable mRNA) species, specifically its rate of translation, can be controllably inhibited by the controllable application of antisense nucleic acids. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

In still another embodiment, RNA aptamers can be introduced into or expressed in a cell. RNA aptamers are specific RNA ligands for proteins, such as for Tat and Rev RNA (Good et al., 1997, Gene Therapy 4: 45-54) that can specifically inhibit their translation.

Post-transcriptional gene silencing (PTGS) or RNA interference (RNAi) can also be used to modify RNA abundances (Guo et al., 1995, Cell 81: 611-620; Fire et al., 1998, Nature 391: 806-811). In RNAi, dsRNAs are injected into cells to specifically block expression of its homologous gene. In particular, in RNAi, both the sense strand and the anti-sense strand can inactivate the corresponding gene. It is suggested that the dsRNAs are cut by nuclease into 21-23 nucleotide fragments. These fragments hybridize to the homologous region of their corresponding mRNAs to form double-stranded segments which are degraded by nuclease (Grant, 1999, Cell 96: 303-306; Tabara et al., 1999, Cell 99: 123-132; Zamore et al., 2000, Cell 101: 25-33; Bass, 2000, Cell 101: 235-238; Petcherski et al., 2000, Nature 405: 364-368; Elbashir et al., Nature 411: 494-498; Paddison et al., Proc. Natl. Acad. Sci. USA 99: 1443-1448; Technical Bulletins at the web site http://www.dharmacon.con/tech/tech03.html, accessed Oct. 16, 2001;). Therefore, in one embodiment, one or more dsRNAs having sequences homologous to the sequences of one or more mRNAs whose abundances are to be modified are transfected into a cell or tissue sample. Any standard method for introducing nucleic acids into cells can be used.

Methods of modifying protein abundances include, inter alia, those altering protein degradation rates and those using antibodies (which bind to proteins affecting abundances of activities of native target protein species). Increasing (or decreasing) the degradation rates of a protein species decreases (or increases) the abundance of that species. Methods for controllably increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature (e.g., 37° C.) and which is hidden to prevent rapid degradation at a lower temperature (e.g., 23° C.) (Dohmen et. al, 1994, Science 263: 1273-1276). Such an exemplary degron is Arg-DHFR^{ts}, a variant of murine dihydrofolate reductase in which the N-terminal Val is replaced by Arg and the Pro at position 66 is replaced with Leu. According to this method, for example, a gene for a target protein, P, is replaced by standard gene targeting methods known in the art (Lodish et al., 1995, Molecular Biology of the Cell, W.H. Freeman and Co., New York, especially chap 8) with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFR^{ts} are not exposed, ubiquitination of the fusion protein does not occur, degradation is slow, and active target protein levels are high. At higher temperatures (in the absence of methotrexate), lysines internal to Arg-DHFR^{ts} are exposed, ubiquitination of the fusion protein occurs, degradation is rapid, and active target protein levels are low. Heat activation of degradation is controllably blocked by exposure methotrexate. This method is adaptable to other N-terminal degrees which are responsive to other inducing factors, such as drugs and temperature changes.

Methods of directly modifying protein activities include, inter alia, dominant negative mutations, specific drugs or chemical moieties generally, and also the use of antibodies.

### 6. EXAMPLE

In the following example an embodiment of the invention in which methods based on gene expression profiles for prediction of drug induced liver damage are described. The example is presented by way of illustration of the present invention, and is not intended to limit the present invention in any way.

I. Material and Experimental Methods

Animal and Sample Preparation

SD rats were treated by 49 hepatotoxic compounds and 10 non-hepatotoxins (Table I) as described previously (see, e.g., Warring et al., 2003, Environ. Health Perspectives 111: 1-8). After 3-day treatment, rat liver samples were collected for RNA extraction and histo-pathology examination. Sera from the same rats were subjected to clinical chemistry monitoring. Table I also summarizes the clinical chemistry information for the 267 rat samples used in this study.

Amplification, Labeling, and Hybridization

Detailed procedures for RNA preparation, amplification, labeling and hybridization are described in, e.g., Hughes et al., 2000, Cell 102: 109-126; Dai et al., 2002, Nucleic Acids Res 30: e86; Roberts et al., 2000, Science 287: 873-880; Waring et al., 2001, Toxicol Appl Pharmacol 175: 28-42; Hughes et al., 2001, Nat. Biotech. 19, 342-347. In brief, total RNA samples were extracted after DNAse treatment. Five micrograms of total RNA from each sample was amplified into cRNA by an in vitro transcription procedure with oligo-dT primer. cRNA was labeled with Cy3 or Cy5 dyes using a two-step process with allylamine-derivatized nucleotides and N-hydroxy succinimide esters of Cy3 or Cy5 (CyDye, Amersham Pharmacia Biotech). The labeled cRNAs were fragmented to an average size of 50-100 nt before hybridization. For each amplified RNA sample, hybridizations were done in duplicate with fluor reversals. After hybridization, slides were washed and scanned using a confocal laser scanner (Agilent Technologies). Fluorescence intensities of the scanned images were quantified, normalized and balanced.

Pooling of Samples

The reference cRNA pool was formed by pooling equal amounts of cRNAs from vehicle treated control samples.

Rat Liver 25K Toxicology Microarray

An in-house custom designed 25K liver toxicology chip was utilized for building the rat liver compendium. Approximate 25,000 probes were selected from ˜50,000 probes based on experimental screening (Warring et al., 2003, Environ. Health Perspectives 111: 1-8). The majority of oligonucleotide probes (˜18,000) selected for the microarray were derived based on a combination of significant differential regulation (P-value <0.2 in any experiment), favorable hybridization kinetics (lower probabilities of cross-hybridization) and biological interest. A smaller proportion of probes (˜6,000) exhibiting lower (but significant) signal intensity in screening experiments were also included.

II. Analytic Method and Result

This methodological study introduces a novel analytic approach for ab initio prediction of hepatotoxicity based on transcriptional profiling. The method comprises two parts. In the first part, a hepatotoxicity score was established to measure the degree of hepatotoxicity. In the second part, a machine learning algorithm and wavelet transformation were employed to build a model for hepatotoxicity estimation. Although the example herein focuses on hepatotoxicity, the approach can be applied to prediction of other types of conditions of a tissue or organ based on transcriptional or other cellular constituent profiles.

Rat Liver Compendium

A rat liver compendium (comprising a database of transcriptional profiles after drug administration) was built with 59 compounds (Table 1). The rat liver toxicology oligonucleotide microarray containing approximately 25,000 probes (Warring et al., 2003, Environ. Health Perspectives 111: 1-8) were employed to build the compendium. Two hundred sixty-seven global transcriptional profiles are included in the current compendium. All profiles in this example come from rats receiving a 3-day treatment. Among the 59 compounds, 49 of them are known liver toxicants. Twenty liver toxicants were administrated with both a low range dose and a high range dose. Twenty toxicants were administrated only with a high range dose. For 10 compounds without any previously observed or reported liver toxicity, both a low dose and high dose range were employed. A detailed list of compounds and associated doses is set forth in Table I.

Overall expression patterns of the liver compendium further confirmed the high reproducibility and sensitivity of the data. Utilizing the combination of error model driven statistics for single chip measurement (Hughes et al., 2000, Cell 102: 109-26; Dai et al., 2002, Nucleic Acids Res 30: e86; Weng, U.S. patent application Ser. No. 10/349,364, filed on Jan. 22, 2003; Weng, U.S. patent application Ser. No. 10/354,664, filed on Jan. 30, 2003, each of which is incorporated by reference herein in its entirety) and the fold changes, there were 2536 genes or reporting ESTs that changed more than 3 fold with p value <0.01 in at least 3 profiles. These were identified as significantly regulated genes in the compendium. Two-dimensional hierarchical clustering was utilized to examine the general expression pattern in the rat liver compendium. The similarity between two profiles x(r) and x(s) was defined as

$$S = 1 - [ {\sum }_{i = 1}^{N} \frac{( {x}_{i} ( r ) - \overline{x} ( r ) )}{{σ}_{{x}_{i}} ( r )} \cdot \frac{( {x}_{i} ( s ) - \overline{x} ( s ) )}{{σ}_{{x}_{i}} ( s )} / \sqrt{{\sum }_{i = 1}^{N} {( \frac{{x}_{i} ( r ) - \overline{x} ( r )}{{σ}_{{x}_{i}} ( r )} )}^{2} \cdot {\sum }_{i = 1}^{N} {( \frac{( {x}_{i} ( s ) - \overline{x} ( s ) )}{{σ}_{{x}_{i}} ( s )} )}^{2}} ]$$

where x(r) and x(s) are two profiles with components of log ratio xᵢ(r) and xᵢ(s); σₓᵢ(r) and σₓᵢ(s) are the estimated errors associated with measured ratios xᵢ(r) and xᵢ(s), respectively; and where i=1, . . . , N; N=2,536, which is the number of measurements in the profiles, e.g., transcriptional profiles, and where

$$\overline{x} ( j ) = {\sum }_{i = 1}^{N} \frac{{x}_{i} ( j )}{{σ}_{{x}_{i}}^{2} ( j )} / {\sum }_{i = 1}^{N} \frac{1}{{σ}_{{x}_{i}}^{2} ( j )}$$

where j=r or s, is the error-weighted arithmetic mean. To emphasize the importance of co-regulation in clustering rather than the amplitude of regulations, the correlation was utilized as a similarity metric. In addition, the set of 2,536 significantly regulated genes were also clustered based on the similarities of their profiles from overall treatments in the compendium. The same similarity metric was used to define the distance, except that for each gene, 267 log ratios across all the treated samples were used to calculate the similarity metric.

The unsupervised 2-dimensional hierarchical clustering demonstrated specific patterns among toxicants and non-hepatotoxins (FIG. 1A). In particular, distinctive expression patterns can be observed between non-hepatotoxins and toxicants. Genes highly regulated by toxicants did not overlap with genes regulated by non-hepatotoxins. Similarity clustering over the compound profile dimension further indicated the large distance between clusters of toxicants and clusters of non-hepatotoxins (FIG. 1A, B). On the other hand, a consistent expression pattern was observed within profiles from rat repeats that received treatments from the same compounds, toxicants or non-hepatotoxins (FIG. 1B, 1C). The observations suggest that transcriptional profiles in the rat compendium contain information for compound hepatotoxicity. The highly reproducible gene expression patterns can be used for compound hepatotoxicity prediction.

Ab Initio Prediction of Hepatotoxicity Based on Transcriptional Profiles

To unveil the hepatotoxicity information embedded in the transcriptional profile, a method was developed to estimate the degree of hepatotoxicity of each individual compound. A set of 212 (80% of 267) profiles with their associated clinical chemistry measurements was selected as a training data set and the remaining 54 (20% of 267) profiles with their associated clinical chemistry measurements were used as a validating data set. An exemplary procedure for ab initio prediction of hepatotoxicity based on transcriptional profiles is illustrated in FIG. 2.

1) Formulation of the Hepatotoxicity Score as a Continual Index for Severity of Hepatotoxicity

A continuous measurement is needed to describe the severity of liver damage for prediction of drug hepatotoxicity. Traditionally, a variety of clinical chemistry measurements (see, e.g., Fogy, 1999, Clinical Chemistry: Principles, Procedures, Correlations, Lippincott Wiliams & Wilkines) and histopathological classification (Zimmerman, 1999, Hepatotoxicity: The adverse effects of drugs and other chemicals on the Liver, Lippincott Williams & Wilkins) have been applied to evaluate liver damage in many different aspects. For example, alanine aminotransferase (ALT) and aspartate aminotransferase (AST) in plasma are used as indicators for hepatocellular injury with a certain degree of specificity. Direct and total bilirubin (Tbil.) measurements in plasma are often utilized to monitor cholestasis. In addition, the histopathological approach provides qualitative evaluation of the liver injury at a cellular level. For example, several types of cellular change, such as necrosis, hypertrophy, steatosis and cholestasis, have been observed from drug induced liver damage. However, due to the complexity of liver injury, the severity of liver damage cannot be sufficiently described by any single one of these indicators.

To reflect the degree of liver damage despite the complicated cellular mechanism of liver injury, a method to combine several traditional clinical measurements into a continual index, the hepatotoxicity score, was developed. Utilizing five clinical chemistry indicators, specifically, ALT, AST, Tbil, alkaline phosphatase (ALP) and cholesterol (Chol), the degree of liver injury resulting from numerous aspects of cellular damage was measured.

I) Formulating a Hepatotoxicity Score Based on Five Clinical Chemistry Measurements

Challenges to formulate a continual measurement that is correlated to the severity of liver damage were the following:
a. The different scales and dynamic range for individual indicators
b. The complicated aspects of individual types of liver damage
c. The lack of a ‘gold standard’ for liver injury.

An approach to overcome the above challenges was developed. First, to meaningfully integrate values of individual clinical chemistry measurements having different scales, the absolute value of each of the individual clinical chemistry measurements was first converted into a distance between the normal value and the actual measured value. It is defined as:
D_{i,j}=(x_{i,j}−μ_{i,0})/σ_{i,0}
In this equation, D_{i,j} is the ith converted clinical chemistry measurement associated with jth profile; i=1,2, . . . 5; j=1, . . . , N; and N=267. The x_{i,j} is the original value of the ith clinical chemistry measurement associated with the jth profile, i=1,2, . . . 5; j=1, . . . , N; and N=267. μ_{i,0} is the average of the ith clinical chemistry measurement in control experiments. σ_{i,0} is the standard deviation of the ith clinical chemistry measurement from a normal control group.

Due to large differences in dynamic ranges among the five converted clinical chemistry measurements, D_{i,j} was further sigmoidally normalized with a different range of linear transformation region according to the following equation

$${D}_{i , j}^{′} = \frac{1 - {ⅇ}^{- {α}_{i , j}}}{1 + {ⅇ}^{- {α}_{i , j}}}$$

$$where$$

$${α}_{i , j} = \frac{{D}_{i , j} - {\overline{D}}_{i}}{{c}_{i} \cdot Std ( {\overline{D}}_{i} )}$$

where D_{i,j} is the ith converted clinical measure of the jth animal, \overline{D}ᵢ is the average of measurements of the ith clinical measure measured from animals in a normal or control group, and std(\overline{D}ᵢ) is the standard derivation of \overline{D}ᵢ, cᵢ is a constant associated with the ith clinical measure, and i=1, 2, . . . , 5, and j=1, 2, . . . , 267. The sigmoidal transformation converts data non-linearly into the range of {−1˜1}. This transformation may retain outliers (abnormal values indicating severe liver damage) without compressing the most commonly occurring values close to the threshold level in treated groups.

With the consideration of different dynamic ranges among those five clinical chemistry indicators and previous knowledge about the sensitivity and specificity in measuring liver injury, the range of linear transformation was adjusted for individual clinical chemistry measurements. For ALT, the most sensitive indicator of liver cell damage, values within 3× standard deviations, instead of 1×, of the average were mapped to the most linear region of the sigmoid. Increasing the range for linear transformation prevents more sub-threshold values from being compressed.

Second, indicators reflecting different aspects of liver damage were employed to construct the hepatotoxicity score. Previous knowledge about the sensitivity and specificity of individual clinical chemistry indicators was integrated into the new indicator. In particular, measurements with higher sensitivity and specificity were given a higher weight when they were integrated into the hepatotoxicity score (HS). Specifically, the hepatotoxicity score was defined as:

$$HS = {D}_{Tbil}^{′} ( if Tbil is abnormal ) + 0.5 {D}_{ALP}^{′} + 3 {D}_{ALT}^{′} + 1.5 {D}_{AST}^{′} + 0.3 {D}_{Chol}^{′} ( if both Chol and least one other clinical measure are abnormal )$$

where contribution from Tbil is zero if Tbil is normal, and the contribution from Chol is zero unless both Chol and at least one of the other clinical measures are abnormal.

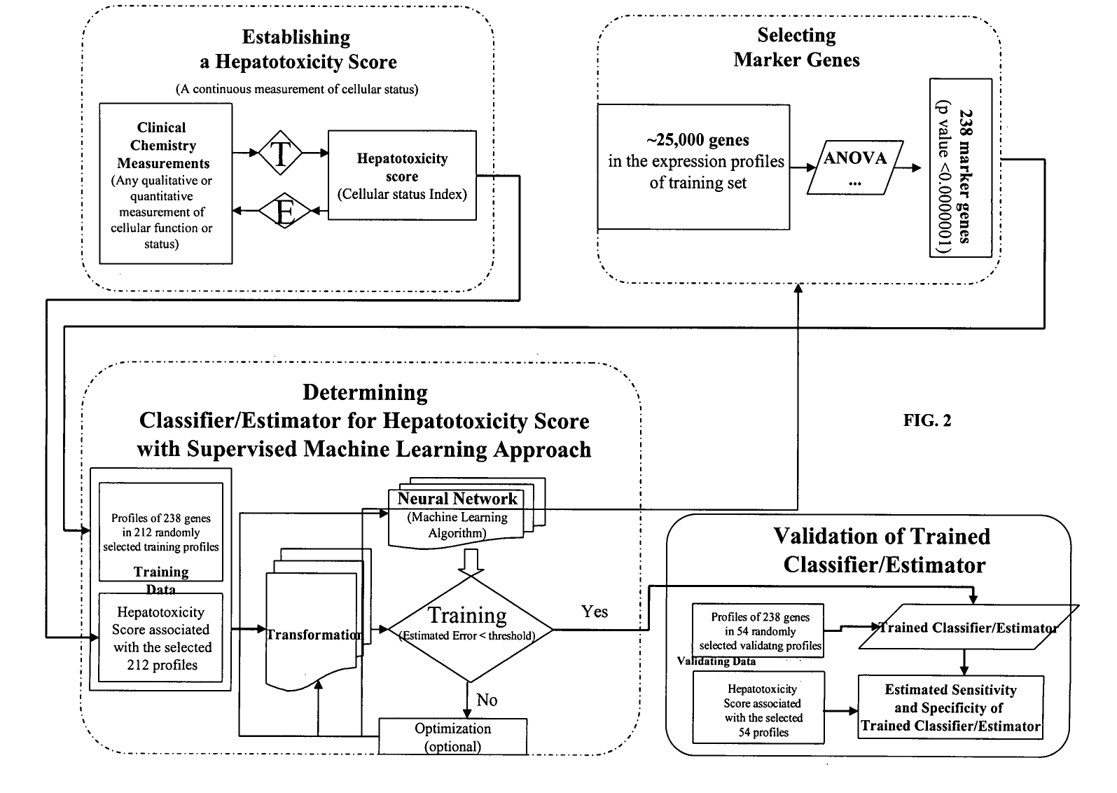

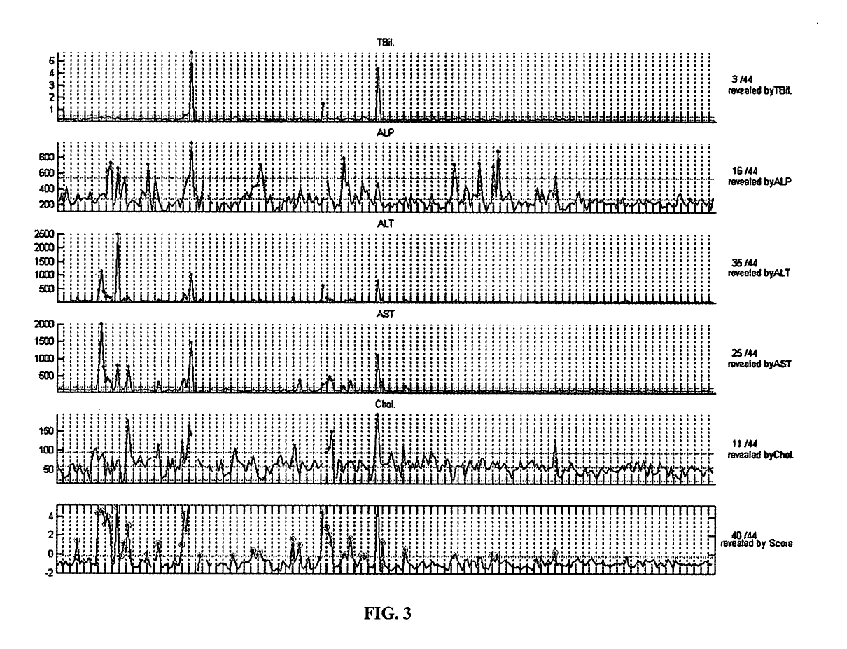

Finally, to evaluate the constructed hepatotoxicity score, the number of false positives and false negatives detected by the hepatotoxicity score was compared with results from each individual clinical chemistry measurement. Liver injury was identified by the individual clinical chemistry measurement if its level was outside +/− two standard deviations of the averaged level of normal animals. Using five clinical chemistry measurements, AST, ALT, ALP, Tbil. and Chol, 44 injured livers from 267 treated rats were detected. Among all these clinical chemistry measures, AST indicated 25, ALT indicated 35, ALP indicated 16, Tbil indicates 3 and Chol indicated 11 liver injuries (FIG. 3). The results from all individual clinical measures are pooled together to form a single ‘gold standard.’ An animal is said to be positive of liver damage if at least one of the individual clinical measure is positive. The results from the HS is then compared with such a gold standard. A false positive by the HS indicates that a positive is identified by the HS but not the gold standard, whereas a false negative by the HS indicates that a negative is identified by the HS but identified as a positive by the gold standard.

ii) Calibrating Threshold for the Hepatotoxicity Score

To have a direct comparison between the traditionally employed clinical chemistry measurements and the established hepatotoxicity score, a threshold indicating liver abnormality based on hepatotoxicity score is used. More importantly, such a threshold can be used for classification of hepatotoxins from unknown compounds, as well.

Utilizing the 44 detected liver injuries as substitute for the ‘gold standard’ in this example, a liver abnormality threshold for hepatotoxicity score was determined by minimizing the false negatives and the sum of false positives and false negatives (FIG. 4). −0.25 was selected as the liver abnormality threshold for the hepatotoxicity score with 4 false negatives and 3 false positives.

iii) Comparing the Sensitivity and Accuracy of the Hepatotoxicity Score to Individual Clinical Chemistry Measurements and Histopathological Data

Comparing the liver damage revealed by the hepatotoxicity score and the liver damage indicated by individual clinical chemistry measures, it can be seen that 90.9% positives were detected with the hepatotoxicity score, whereas only 56.8% positives were detected with AST, 79.5% positives with ALT, 36% positives with ALP, 6.8% positives with Tbil and 25% with Chol. (FIG. 5A-5C). The increasing sensitivity of the hepatotoxicity score suggests the power of combining individual clinical chemistry measures because each individual measure, such as ALT and Tbil, is most sensitive to only a certain aspect of liver damage. For example, in the high dose experiment group, liver damage was only reported by ALT in the No.2 rat that received perhexilene (320 mg/kg/day). Another case of liver abnormality was only detected by AST in the No. 1 rat received ethanol (3000 mg/kg/day) treatment. Such abnormality would not be detected with either AST or ALT alone. However, as a combination of all five clinical chemistry measures, the hepatotoxicity score is more sensitive in detecting liver abnormality.

To further evaluate the accuracy of the hepatotoxicity score, the histopathological examination results from the four false negatives and three false positives detected by the hepatotoxicity score were investigated (FIG. 5A-B and 5C). Among the four false negatives, one received metformin, a non-hepatotoxicin (900 mg/kg/day), two received low dose hepatotoxins, TNF-alpha at 0.01 mg/kg/day and tamoxifen at 5 mg/kg/day, and one received a high dose hepatotoxin, monocrotaline at 50 mg/kg/day treatment. Histopathology examination reported no observable liver abnormality in those four samples. This observation suggests that the specificity of the hepatotoxicity score was higher than that of the individual clinical chemistry measurements. Among the three false positive cases, all of them belonged to the high dose hepatotoxin-treated group. Specifically, the positives detected by the hepatotoxicity score, but not by any of the clinical chemistry measures, include the estradiol glucuronide (10 mg/kg/day) and aspirin (150 mg/kg/day) treated groups. Cellular abnormality was reported in both treatment groups. Among the three estradiol glucuronide treated rats, the other two showed liver injury by ALT and ALP, as well. The evidence from histopathology examination and clinical chemistry measurements for other members of these treated rats suggest that the hepatotoxicity score is sensitive enough to reveal a mild degree of liver injury that cannot be detected by any single clinical chemistry measurement. Hence, the hepatotoxicity score was a comprehensive indicator of the degree of liver damage with reliable specificity and sensitivity. It can be utilized to estimate the degree of liver damage in response to compounds.

2) Selection of Marker Genes for Ab Initio Prediction of Hepatotoxicity Score

Marker genes for ab initio prediction of hepatotoxicity score were selected from the training data set of 212 profiles by use of an ANOVA approach. Those 238 genes significantly regulated between the non-hepatotoxin and the hepatotoxin treated groups were chosen by their error weighted log ratio (p value <0.0000001). The error weighted log ratio is defined as

$${Xdev}_{i} = \frac{log {x}_{i}}{{σ}_{log {x}_{i}}}$$

where Xdevᵢ is the error, i.e., σ_{log x}_{ᵢ}, weighted log ratio of the ith measurement, xᵢ; i=1, 2, . . . , N; and N is the number of measurements in the profile. The 238 marker genes are listed in Table II.

To reduce the number of dimensions while to keep the expression regulation information, the Xdev for each significantly regulated gene was transformed by a wavelet transformation with Daubechies wavelet function at a level of 5. The transformation retained the main regulation information across treatment groups for each individual gene, but reduced the variable dimension to 31. With a reduced parameter dimension, there was less chance that the model obtained from the current 212 profiles in the training data set was over-fitted.

3) Establishment of a Model for Ab Initio Prediction of Hepatotoxicity Score Based on Selected Marker Genes Using Neural Networks (NN)

A model for hepatotoxicity score prediction was obtained from the transformed training data set with 212 expression profiles and their associated hepatotoxicity scores by an artificial neural network (Bishop,1995, Neural networks for pattern recognition (Oxford, Clarendon); and Nabney, 2001, Netlab: Algorithms for pattern recognition (London, Springer)). The 31 transformed variables from the 238 reporter genes were utilized as independent variables, i.e., as input for the neural network. The hepatotoxicity scores associated with individual profiles were treated as a dependent variable, i.e., the output of the neural network. The multi-layer perceptron (MLP) was employed as the architecture of the neural network. The optimal model structure was determined by a cross-validating sampling approach. In particular, 80% of the 212 profiles were randomly chosen and utilized as a training set and the rest of 20% of 212 profiles were used to determine the estimated error associated with certain neural network structure. A neural network structure with one hidden layer of 15 units demonstrated the lowest estimated error rate. An example of prediction from this trained neural network is illustrated (FIG. 6A). At the top panel, profiles are arranged according to their associated hepatotoxicity score, demonstrated by the filed dots. The predicted hepatotoxicity scores obtained from the trained model with an optimal structure are shown as unfilled squares. Prediction error is estimated by the average of the deviation between the expected value and the predicted value of the hepatotoxicity from the 212 profiles. The estimated error for prediction from the training set was 0.08.

The specificity and sensitivity of the trained model in the training data set were further examined with the pre-established liver hepatotoxicity score threshold (FIGS. 7A-B). Utilizing the positives (indicated as EP) detected by the combination of five clinical chemistry measurements as a substitute for the gold standard, the numbers of false positives (FP) and false negatives (FN) were determined by comparing the positives obtained from prediction (PP) with the EP. Among the 212 profiles, the model reported 12 false positives and 10 false negatives with 89.6% of prediction accuracy.

3) Validation of Trained NN Model with an Independent Data Set

The accuracy and generality of the trained model was examined by an independent data set with 54 expression profiles (FIG. 6B and FIG. 8). At the bottom panel of FIG. 6, profiles are arranged according to their associated hepatotoxicity score, demonstrated by the filled dots. Shown as unfilled squares, the hepatotoxicity scores were predicted from the previously trained model with data from the validating data set. Prediction error was estimated by the average of the deviation between the expected value and the predicted value based on the 54 profiles. The estimated error for prediction from the validating data set was 0.632.

To determine whether the error associated with the trained model was significantly different from random error, 5000 times of Monte Carlo simulation were conducted and the estimated error from random distribution was 1.177, significantly higher than the error associated with the trained model (p value <0.05).

The specificity and sensitivity of the trained model in the validating data set were further confirmed with the pre-established liver hepatotoxicity score threshold (FIG. 8). Utilizing the positives (indicated as EP) detected by the combination of five clinical chemistry measurements of those 54 profiles as a gold standard, the number of FP and the number of FN were determined by comparing the positives obtained from PP with the EP. Among the 54 profiles, the model reported 5 false positives and 1 false negative with 88% of prediction accuracy.

To further evaluate the accuracy of the model, pathological observations in the 5 false positives and 1 false negatives from the validating data set were also carried out. The five false positives detected by the trained model include profiles from No. 1 rat receiving dimethlformamide (1000 mg/kg/day), No.1 rat receiving tetracycline (500 mg/kg/day), No,3 rat receiving diethylnitrosamine (100 mg/kg/day), No.1 rat receiving L-ethionine(50 mg/kg/day) and levofloxain (200 mg/kg/day). Although among them, levofloxacin is a non-hepatotoxin, noticeable pathological changes were discovered in the rest of the four compound-treated groups. Further optimization of reporter genes and transformation may help to eliminate the mistakenly classified levofloxacin and the false negative iodoacetic acid profile.

In summary, a model for ab initio prediction of hepatotoxicity based on 238 marker genes was established by 212 transcriptional profiles and validated with 54 independent transcriptional profiles. The comparable accuracy revealed by both the training data set and validating data set indicates high generality for the trained prediction model in this example. This suggests a good coverage of the toxicity represented by the training data set and the adequate sample size in the training data set. Hence, the gene set and the associated model derived from our method can be utilized to predict hepatotoxicity of unknown compounds with specified accuracy.

III. Discussion

The analytic approach developed in this example for the ab initio prediction of hepatotoxicity based on transcriptional profile has important utilities and implications in, among others, drug discovery and basic biological research. In the toxicogenomics field, it is the first method that enables drug toxicity to be ab initio predicted in a quantitative manner. Unlike any other analytical approaches applied in the field of toxicogenomics, the accuracy and generality of the prediction model based on transcriptional profiles can be ascertained.

Utility and Implication of 238 Hepatotoxicity Score Marker Genes from Rat Liver Toxic Compendium and the Associated Estimation Model

The generality and accuracy of the trained model indicates that the model and the associated 238 marker genes can be applied to hepatotoxicity prediction for drug screening. If a rat compendium comprising more profiles covering results from more drugs and/or doses is used, the sensitivity and specificity may be further improved.

More importantly, the set of markers and the trained model can be used to evaluate the currently used in vitro cell culture system for hepatotoxicity prediction. It has been debated for a long time whether hepatocyte cultures can represent the degree of hepatotoxicity revealed in rat liver. Associated with this debate is which cell culture system best reflects the response of liver to hepatotoxin treatment. Based on the 238 marker genes and the trained model, whether any cultured hepatocytes or cell lines can represent a similar degree of toxic response induced by those compounds in liver is determined. In addition, for those cell cultures which mimic the degree of toxic response induced by anchor compounds in rat liver, the in vitro concentration of those compounds at which those compounds exhibit equivalent toxicity in rat liver is determined. Thus, a correlation between the in vitro and in vivo concentration is determined.

The method described in this example is not limited to toxicity prediction. A number of modifications can be applied for specific implementations. For example, there are a variety of different implementations for selecting and optimizing marker genes, transforming original data and establishing a model or classifier. Although the artificial neural network algorithm is utilized in this example, other modeling methods can also be used. Other supervised machine learning algorithms, such as Bayesian network and supporting vector machine, can also be applied on modeling the association between the hepatotoxicity score and the transcriptional profiles. An exhaustive optimization of the marker genes and associated transformation is also applied. Such an optimization further improves the accuracy of the prediction model.

Utility and Implication for Our Approach to Build Index for Cellular Function and Cellular Status

The approach of this example to combine multiple measurements for hepatotoxicities into one continual index for liver toxicity was used for ab initio predicting the degree of liver toxicity based on transcriptional profiles. In this example, five clinical chemistry measurements were utilized to build the hepatotoxicity score. When even more traditional clinical measures for hepatotoxicity are incorporated into the hepatotoxicity score, the sensitivity of the integrated measurement to the complicated toxic response in liver may be further enhanced. Moreover, the measurements that can be used to build the index are not limited to quantitative clinical measures. Qualitative clinical measures, such as categorical indicators like grade of pathological abnormalities, can also be combined into the composite clinical score. The approach can also be used to establish new clinical scores to measure other function and/or status of cells. For example, the composite clinical score can be used to monitor the degree of tumorigenesis, or the potential for cancer cells to survive certain chemotherapy. Such scores can be used to quantitatively characterize cellular changes associated with multiple aspects of complicated cellular events and to uncover such complicated changes as liver damage or tumorigenesis based on transcriptional profiles or proteomics data.

TABLE I
Compounds and doses associated with the liver toxicity compendium data. For each
compound and each dosage, up to three rats were tested. Different rats tested with each
compound/dosage are identified by a Rat ID. Clinical chemistry measurements for the 267
profiles are also listed.

| | | | Rats | | | | | |
|---|---|---|---|---|---|---|---|---|
| Compound | Dose | | ID | TBil | ALP | ALT | AST | Chol. |
| 3-methylcholanthrene | 100 | mg/kg/day | 1 | 0.300 | 216.000 | 39.000 | 104.000 | 101.000 |
| 3-methylcholanthrene | 100 | mg/kg/day | 2 | 0.300 | 234.000 | 27.000 | 131.000 | 106.000 |
| 3-methylcholanthrene | 100 | mg/kg/day | 3 | 0.300 | 192.000 | 32.000 | 122.000 | 80.000 |
| acetominophen | 70 | mg/kg/day | 1 | 0.100 | 164.000 | 36.000 | 87.000 | 65.000 |
| acetominophen | 70 | mg/kg/day | 2 | 0.100 | 223.000 | 37.000 | 107.000 | 50.000 |
| acetominophen | 70 | mg/kg/day | 3 | 0.100 | 177.000 | 31.000 | 74.000 | 53.000 |
| acetominophen | 700 | mg/kg/day | 1 | 0.200 | 193.000 | 36.000 | 89.000 | 78.000 |
| acetominophen | 700 | mg/kg/day | 2 | 0.200 | 342.000 | 40.000 | 86.000 | 73.000 |
| acetominophen | 700 | mg/kg/day | 3 | 0.200 | 177.000 | 29.000 | 97.000 | 74.000 |
| actinomycin D | 0.4 | mg/kg/day | 1 | 0.200 | 284.000 | 100.000 | 485.000 | 108.000 |
| actinomycin D | 0.4 | mg/kg/day | 2 | 0.300 | 309.000 | 71.000 | 324.000 | 121.000 |
| actinomycin D | 0.4 | mg/kg/day | 3 | 0.300 | 272.000 | 75.000 | 376.000 | 147.000 |
| adriamycin | 1 | mg/kg/day | 1 | 0.200 | 413.000 | 36.000 | 80.000 | 53.000 |
| adriamycin | 1 | mg/kg/day | 2 | 0.200 | 387.000 | 38.000 | 66.000 | 58.000 |
| adriamycin | 1 | mg/kg/day | 3 | 0.200 | 296.000 | 27.000 | 66.000 | 69.000 |
| adriamycin | 10 | mg/kg/day | 1 | 0.200 | 156.000 | 67.000 | 285.000 | 118.000 |
| adriamycin | 10 | mg/kg/day | 2 | 0.200 | 220.000 | 139.000 | 749.000 | 175.000 |
| adriamycin | 10 | mg/kg/day | 3 | 0.300 | 240.000 | 78.000 | 319.000 | 112.000 |
| aflatoxin B | 0.5 | mg/kg/day | 1 | 0.200 | 297.000 | 81.000 | 206.000 | 64.000 |
| aflatoxin B | 0.5 | mg/kg/day | 2 | 0.200 | 166.000 | 48.000 | 132.000 | 75.000 |
| aflatoxin B | 0.5 | mg/kg/day | 3 | 0.200 | 217.000 | 59.000 | 156.000 | 63.000 |
| allopurinol | 100 | mg/kg/day | 1 | 0.200 | 199.000 | 33.000 | 86.000 | 60.000 |
| allopurinol | 100 | mg/kg/day | 2 | 0.200 | 210.000 | 42.000 | 100.000 | 68.000 |
| allopurinol | 100 | mg/kg/day | 3 | 0.200 | 265.000 | 34.000 | 83.000 | 58.000 |
| allyl alcohol | 40 | mg/kg/day | 1 | 0.300 | 300.000 | 425.000 | 660.000 | 71.000 |
| allyl alcohol | 40 | mg/kg/day | 2 | 0.400 | 326.000 | 1130.000 | 2010.000 | 85.000 |
| allyl alcohol | 40 | mg/kg/day | 3 | 0.300 | 344.000 | 380.000 | 750.000 | 92.000 |
| amiodarone | 10 | mg/kg/day | 1 | 0.300 | 308.000 | 43.000 | 117.000 | 63.000 |
| amiodarone | 10 | mg/kg/day | 2 | 0.300 | 221.000 | 46.000 | 140.000 | 54.000 |
| amiodarone | 10 | mg/kg/day | 2 | 0.300 | 221.000 | 46.000 | 140.000 | 54.000 |
| amiodarone | 10 | mg/kg/day | 3 | 0.300 | 287.000 | 39.000 | 128.000 | 64.000 |
| amiodarone | 100 | mg/kg/day | 1 | 0.200 | 319.000 | 33.000 | 122.000 | 87.000 |
| amiodarone | 100 | mg/kg/day | 2 | 0.200 | 178.000 | 30.000 | 104.000 | 72.000 |
| amiodarone | 100 | mg/kg/day | 3 | 0.200 | 222.000 | 27.000 | 108.000 | 62.000 |
| ANIT | 40 | mg/kg/day | 1 | 0.800 | 561.000 | 430.000 | 700.000 | 162.000 |
| ANIT | 40 | mg/kg/day | 2 | 5.700 | 991.000 | 1000.000 | 1460.000 | 141.000 |
| ANIT | 40 | mg/kg/day | 3 | 4.400 | 485.000 | 800.000 | 1100.000 | 194.000 |
| Aroclor | 100 | mg/kg/day | 1 | 0.200 | 309.000 | 46.000 | 109.000 | 62.000 |
| Aroclor | 100 | mg/kg/day | 2 | 0.500 | 186.000 | 45.000 | 108.000 | 61.000 |
| Aroclor | 100 | mg/kg/day | 3 | 0.200 | 269.000 | 30.000 | 101.000 | 58.000 |
| Aroclor 1254 | 400 | mg/kg/day | 1 | 0.300 | 251.000 | 29.000 | 111.000 | 70.000 |
| Aroclor 1254 | 400 | mg/kg/day | 2 | 0.200 | 206.000 | 39.000 | 135.000 | 77.000 |
| Aroclor 1254 | 400 | mg/kg/day | 3 | 0.300 | 213.000 | 42.000 | 106.000 | 76.000 |
| Arsenic | 20 | mg/kg/day | 1 | 0.200 | 224.000 | 25.000 | 65.000 | 52.000 |
| Arsenic | 20 | mg/kg/day | 2 | 0.200 | 253.000 | 24.000 | 83.000 | 71.000 |
| Arsenic | 20 | mg/kg/day | 3 | 0.200 | 314.000 | 20.000 | 83.000 | 72.000 |
| Aspirin | 15 | mg/kg/day | 1 | 0.300 | 351.000 | 51.000 | 77.000 | 57.000 |
| Aspirin | 15 | mg/kg/day | 2 | 0.300 | 428.000 | 49.000 | 109.000 | 68.000 |
| Aspirin | 15 | mg/kg/day | 3 | 0.300 | 383.000 | 54.000 | 96.000 | 59.000 |
| Aspirin | 150 | mg/kg/day | 1 | 0.500 | 494.000 | 52.000 | 89.000 | 69.000 |
| Aspirin | 150 | mg/kg/day | 2 | 0.400 | 350.000 | 46.000 | 94.000 | 61.000 |
| Aspirin | 150 | mg/kg/day | 3 | 0.300 | 395.000 | 62.000 | 109.000 | 53.000 |
| bezafibrate | 20 | mg/kg/day | 1 | 0.200 | 147.000 | 50.000 | 130.000 | 33.000 |
| bezafibrate | 20 | mg/kg/day | 2 | 0.200 | 246.000 | 32.000 | 111.000 | 31.000 |
| bezafibrate | 20 | mg/kg/day | 3 | 0.400 | 156.000 | 33.000 | 101.000 | 38.000 |
| bezafibrate | 200 | mg/kg/day | 1 | 0.200 | 216.000 | 32.000 | 156.000 | 38.000 |
| bezafibrate | 200 | mg/kg/day | 2 | 0.200 | 212.000 | 35.000 | 134.000 | 33.000 |
| bezafibrate | 200 | mg/kg/day | 3 | 0.400 | 206.000 | 29.000 | 96.000 | 31.000 |
| carbamazepine | 250 | mg/kg/day | 1 | 0.200 | 251.000 | 49.000 | 95.000 | 38.000 |
| carbamazepine | 250 | mg/kg/day | 2 | 0.200 | 220.000 | 51.000 | 128.000 | 59.000 |
| carbamazepine | 250 | mg/kg/day | 3 | 0.200 | 329.000 | 48.000 | 102.000 | 39.000 |
| carbamazepine | 50 | mg/kg/day | 1 | 0.200 | 167.000 | 34.000 | 80.000 | 76.000 |
| carbamazepine | 50 | mg/kg/day | 2 | 0.200 | 370.000 | 39.000 | 104.000 | 49.000 |
| carbamazepine | 50 | mg/kg/day | 3 | 0.200 | 225.000 | 42.000 | 90.000 | 62.000 |
| carbon tetrachloride | 1000 | mg/kg/day | 1 | 0.300 | 273.000 | 217.000 | 328.000 | 26.000 |
| carbon tetrachloride | 1000 | mg/kg/day | 2 | 0.500 | 632.000 | 198.000 | 420.000 | 69.000 |
| carbon tetrachloride | 1000 | mg/kg/day | 3 | 0.300 | 728.000 | 152.000 | 343.000 | 38.000 |
| chlorpheniramine | 1.9 | mg/kg | 1 | 0.100 | 268.000 | 47.000 | 125.000 | 49.000 |
| chlorpheniramine | 1.9 | mg/kg | 2 | 0.100 | 232.000 | 56.000 | 166.000 | 50.000 |
| chlorpheniramine | 1.9 | mg/kg | 3 | 0.100 | 282.000 | 42.000 | 105.000 | 40.000 |
| chlorpheniramine | 19 | mg/kg | 1 | 0.100 | 255.000 | 39.000 | 83.000 | 27.000 |
| chlorpheniramine | 19 | mg/kg | 2 | 0.100 | 238.000 | 38.000 | 111.000 | 48.000 |
| chlorpheniramine | 19 | mg/kg | 3 | 0.100 | 271.000 | 35.000 | 111.000 | 57.000 |
| chlorpromazine | 100 | mg/kg/day | 1 | 0.400 | 352.000 | 38.000 | 131.000 | 122.000 |
| chlorpromazine | 100 | mg/kg/day | 2 | 0.300 | 710.000 | 56.000 | 121.000 | 73.000 |
| chlorpromazine | 100 | mg/kg/day | 3 | 0.200 | 381.000 | 42.000 | 106.000 | 80.000 |
| chlorpromazine | 25 | mg/kg/day | 1 | 0.400 | 683.000 | 57.000 | 108.000 | 75.000 |
| chlorpromazine | 25 | mg/kg/day | 2 | 0.200 | 377.000 | 58.000 | 127.000 | 66.000 |
| chlorpromazine | 25 | mg/kg/day | 3 | 0.200 | 884.000 | 53.000 | 106.000 | 73.000 |
| cycloheximide | 1 | mg/kg/day | 1 | 0.200 | 133.000 | 47.000 | 65.000 | 26.000 |
| cycloheximide | 1 | mg/kg/day | 2 | 0.200 | 119.000 | 40.000 | 63.000 | 33.000 |
| cycloheximide | 1 | mg/kg/day | 3 | 0.200 | 128.000 | 50.000 | 65.000 | 33.000 |
| cyclophosphamide | 100 | mg/kg/day | 1 | 0.200 | 173.000 | 20.000 | 61.000 | 98.000 |
| cyclophosphamide | 100 | mg/kg/day | 2 | 0.200 | 188.000 | 25.000 | 75.000 | 75.000 |
| cyclophosphamide | 100 | mg/kg/day | 3 | 0.300 | 202.000 | 24.000 | 85.000 | 111.000 |
| dexamethasone | 1 | mg/kg/day | 1 | 0.300 | 335.000 | 63.000 | 98.000 | 81.000 |
| dexamethasone | 1 | mg/kg/day | 2 | 0.400 | 558.000 | 60.000 | 64.000 | 123.000 |
| dexamethasone | 1 | mg/kg/day | 3 | 0.200 | 302.000 | 56.000 | 87.000 | 69.000 |
| dexamethasone | 10 | mg/kg/day | 1 | 0.300 | 437.000 | 78.000 | 132.000 | 87.000 |
| dexamethasone | 10 | mg/kg/day | 2 | 0.300 | 311.000 | 145.000 | 125.000 | 93.000 |
| dexamethasone | 10 | mg/kg/day | 3 | 0.200 | 157.000 | 60.000 | 67.000 | 116.000 |
| diazepam | 3.6 | mg/kg | 1 | 0.100 | 242.000 | 40.000 | 110.000 | 37.000 |
| diazepam | 3.6 | mg/kg | 3 | 0.100 | 199.000 | 56.000 | 103.000 | 53.000 |
| diazepam | 36 | mg/kg | 1 | 0.100 | 255.000 | 39.000 | 120.000 | 55.000 |
| diazepam | 36 | mg/kg | 2 | 0.100 | 142.000 | 45.000 | 82.000 | 57.000 |
| diazepam | 36 | mg/kg | 3 | 0.100 | 304.000 | 39.000 | 87.000 | 44.000 |
| diclofenac | 10 | mg/kg day | 3 | 0.200 | 227.000 | 26.000 | 76.000 | 53.000 |
| diclofenac | 10 | mg/kg/day | 1 | 0.100 | 249.000 | 32.000 | 75.000 | 56.000 |
| diclofenac | 10 | mg/kg/day | 2 | 0.200 | 257.000 | 21.000 | 70.000 | 80.000 |
| diclofenac | 40 | mg/kg/day | 1 | 0.200 | 796.000 | 38.000 | 209.000 | 43.000 |
| diclofenac | 40 | mg/kg/day | 2 | 0.400 | 421.000 | 62.000 | 379.000 | 37.000 |
| diclofenac | 40 | mg/kg/day | 3 | 0.100 | 137.000 | 22.000 | 48.000 | 76.000 |
| diethylnitrosamine | 100 | mg/kg/day | 1 | 0.200 | 194.000 | 28.000 | 84.000 | 53.000 |
| diethylnitrosamine | 100 | mg/kg/day | 2 | 0.200 | 163.000 | 34.000 | 132.000 | 52.000 |
| diethylnitrosamine | 100 | mg/kg/day | 3 | 0.200 | 214.000 | 33.000 | 113.000 | 67.000 |
| diethylstilbestrol | 25 | mg/kg/day | 1 | 0.200 | 416.000 | 15.000 | 56.000 | NaN |
| diethylstilbestrol | 25 | mg/kg/day | 2 | 0.200 | 310.000 | 27.000 | 96.000 | NaN |
| diethylstilbestrol | 25 | mg/kg/day | 3 | 0.200 | 531.000 | 16.000 | 56.000 | NaN |
| dimethylformamide | 1000 | mg/kg/day | 1 | 0.300 | 256.000 | 40.000 | 100.000 | 84.000 |
| dimethylformamide | 1000 | mg/kg/day | 2 | 0.200 | 261.000 | 42.000 | 123.000 | 74.000 |
| dimethylformamide | 1000 | mg/kg/day | 3 | 0.400 | 666.000 | 2500.000 | 790.000 | 74.000 |
| dinitrophenol | 25 | mg/kg/day | 1 | 0.100 | 215.000 | 22.000 | 72.000 | 54.000 |
| dinitrophenol | 25 | mg/kg/day | 2 | 0.200 | 309.000 | 28.000 | 70.000 | 72.000 |
| dinitrophenol | 25 | mg/kg/day | 3 | 0.200 | 214.000 | 27.000 | 69.000 | 64.000 |
| Diquat | 68.8 | mg/kg/day | 1 | 0.200 | 129.000 | 42.000 | 94.000 | 49.000 |
| Diquat | 68.8 | mg/kg/day | 2 | 0.300 | 111.000 | 32.000 | 80.000 | 81.000 |
| DNP | 5 | mg/kg/day | 1 | 0.200 | 218.000 | 24.000 | 62.000 | 73.000 |
| DNP | 5 | mg/kg/day | 2 | 0.200 | 167.000 | 39.000 | 68.000 | 58.000 |
| DNP | 5 | mg/kg/day | 3 | 0.200 | 230.000 | 27.000 | 77.000 | 54.000 |
| erythromycin | 80 | mg/kg/day | 1 | 0.300 | 143.000 | 35.000 | 116.000 | 48.000 |
| erythromycin | 80 | mg/kg/day | 2 | 0.300 | 118.000 | 33.000 | 111.000 | 51.000 |
| erythromycin | 80 | mg/kg/day | 3 | 0.200 | 136.000 | 43.000 | 101.000 | 54.000 |
| erythromycin | 800 | mg/kg/day | 1 | 0.400 | 236.000 | 46.000 | 117.000 | 87.000 |
| erythromycin | 800 | mg/kg/day | 2 | 0.500 | 233.000 | 42.000 | 109.000 | 105.000 |
| erythromycin | 800 | mg/kg/day | 3 | 0.300 | 223.000 | 70.000 | 123.000 | 83.000 |
| estradiol | 1 | mg/kg/day | 1 | 0.200 | 332.000 | 52.000 | 70.000 | 51.000 |
| estradiol | 1 | mg/kg/day | 2 | 0.200 | 315.000 | 46.000 | 75.000 | 52.000 |
| estradiol | 1 | mg/kg/day | 3 | 0.300 | 542.000 | 64.000 | 70.000 | 60.000 |
| estradiol glucuronide | 10 | mg/kg/day | 1 | 0.300 | 360.000 | 54.000 | 104.000 | 28.000 |
| estradiol glucuronide | 10 | mg/kg/day | 2 | 0.200 | 416.000 | 61.000 | 106.000 | 17.000 |
| estradiol glucuronide | 10 | mg/kg/day | 3 | 0.200 | 541.000 | 102.000 | 159.000 | 25.000 |
| Ethanol | 3000 | mg/kg/day | 1 | 0.300 | 440.000 | 64.000 | 109.000 | 65.000 |
| Ethanol | 3000 | mg/kg/day | 2 | 0.300 | 487.000 | 49.000 | 81.000 | 68.000 |
| etoposide | 50 | mg/kg/day | 1 | 0.200 | 280.000 | 35.000 | 108.000 | 78.000 |
| etoposide | 50 | mg/kg/day | 2 | 0.200 | 256.000 | 27.000 | 72.000 | 79.000 |
| etoposide | 50 | mg/kg/day | 3 | 0.200 | 259.000 | 27.000 | 84.000 | 62.000 |
| HPMC | 0.2% | | 1 | 0.100 | 280.000 | 39.000 | 147.000 | 50.000 |
| HPMC | 0.2% | | 2 | 0.100 | 371.000 | 50.000 | 126.000 | 39.000 |
| HPMC | 0.2% | | 3 | 0.100 | 224.000 | 44.000 | 107.000 | 37.000 |
| ibuprofen | 20 | mg/kg/day | 1 | 0.200 | 234.000 | 32.000 | 83.000 | 60.000 |
| ibuprofen | 20 | mg/kg/day | 2 | 0.200 | 193.000 | 24.000 | 82.000 | 77.000 |
| ibuprofen | 20 | mg/kg/day | 3 | 0.200 | 234.000 | 31.000 | 92.000 | 70.000 |
| ibuprofen | 200 | mg/kg/day | 1 | 0.200 | 229.000 | 23.000 | 88.000 | 70.000 |
| ibuprofen | 200 | mg/kg/day | 2 | 0.200 | 114.000 | 26.000 | 71.000 | 83.000 |
| ibuprofen | 200 | mg/kg/day | 3 | 0.200 | 145.000 | 23.000 | 64.000 | 89.000 |
| indomethacin | 20 | mg/kg/day | 1 | 0.200 | 218.000 | 44.000 | 95.000 | 49.000 |
| indomethacin | 20 | mg/kg/day | 2 | 0.200 | 215.000 | 28.000 | 83.000 | 66.000 |
| indomethacin | 20 | mg/kg/day | 3 | 0.200 | 171.000 | 36.000 | 104.000 | 65.000 |
| iodoacetic acid | 50 | mg/kg/day | 1 | 0.300 | 186.000 | 100.000 | 327.000 | 63.000 |
| iodoacetic acid | 50 | mg/kg/day | 2 | 0.300 | 219.000 | 115.000 | 343.000 | 73.000 |
| iodoacetic acid | 50 | mg/kg/day | 3 | 0.200 | 222.000 | 78.000 | 142.000 | 70.000 |
| ketoconozole | 120 | mg/kg/day | 1 | 0.300 | 272.000 | 74.000 | 84.000 | 68.000 |
| ketoconozole | 120 | mg/kg/day | 2 | 0.200 | 481.000 | 51.000 | 99.000 | 54.000 |
| ketoconozole | 120 | mg/kg/day | 3 | 0.200 | 440.000 | 54.000 | 85.000 | 58.000 |
| L-ethionine | 50 | mg/kg/day | 1 | 0.200 | 274.000 | 34.000 | 77.000 | 56.000 |
| L-ethionine | 50 | mg/kg/day | 2 | 0.200 | 261.000 | 28.000 | 82.000 | 25.000 |
| L-ethionine | 50 | mg/kg/day | 3 | 0.200 | 212.000 | 22.000 | 84.000 | 32.000 |
| levofloxacin | 20 | mg/kg | 1 | 0.100 | 236.000 | 46.000 | 116.000 | 50.000 |
| levofloxacin | 20 | mg/kg | 2 | 0.100 | 209.000 | 32.000 | 109.000 | 44.000 |
| levofloxacin | 20 | mg/kg | 3 | 0.100 | 222.000 | 40.000 | 80.000 | 53.000 |
| levofloxacin | 200 | mg/kg | 1 | 0.100 | 194.000 | 34.000 | 72.000 | 58.000 |
| levofloxacin | 200 | mg/kg | 2 | 0.100 | 236.000 | 35.000 | 100.000 | 49.000 |
| levofloxacin | 200 | mg/kg | 3 | 0.100 | 166.000 | 44.000 | 89.000 | 49.000 |
| metformin | 90 | mg/kg | 1 | 0.100 | 275.000 | 40.000 | 100.000 | 45.000 |
| metformin | 90 | mg/kg | 2 | 0.100 | 222.000 | 32.000 | 117.000 | 54.000 |
| metformin | 90 | mg/kg | 3 | 0.100 | 299.000 | 41.000 | 93.000 | 63.000 |
| metformin | 900 | mg/kg | 1 | 0.100 | 164.000 | 36.000 | 118.000 | 50.000 |
| metformin | 900 | mg/kg | 2 | 0.100 | 229.000 | 65.000 | 110.000 | 67.000 |
| metformin | 900 | mg/kg | 3 | 0.100 | 187.000 | 34.000 | 85.000 | 71.000 |
| methapyrilene | 50 | mg/kg/day | 1 | 0.200 | 211.000 | 36.000 | 154.000 | 46.000 |
| methapyrilene | 50 | mg/kg/day | 2 | 0.200 | 253.000 | 36.000 | 94.000 | 52.000 |
| methapyrilene | 50 | mg/kg/day | 3 | 0.200 | 343.000 | 57.000 | 141.000 | 50.000 |
| methotrexate | 250 | mg/kg/day | 1 | 0.200 | 141.000 | 24.000 | 70.000 | 57.000 |
| methotrexate | 250 | mg/kg/day | 2 | 0.200 | 126.000 | 16.000 | 85.000 | 65.000 |
| methotrexate | 250 | mg/kg/day | 3 | 0.200 | 167.000 | 25.000 | 91.000 | 64.000 |
| microcystin | 0.05 | mg/kg/day | 1 | 0.600 | 518.000 | 140.000 | 204.000 | 84.000 |
| microcystin | 0.05 | mg/kg/day | 2 | 0.600 | 427.000 | 258.000 | 384.000 | 67.000 |
| microcystin | 0.05 | mg/kg/day | 3 | 0.400 | 514.000 | 144.000 | 296.000 | 95.000 |
| minoxidil | 3 | mg/kg | 1 | 0.100 | 189.000 | 35.000 | 100.000 | 50.000 |
| minoxidil | 3 | mg/kg | 2 | 0.100 | 206.000 | 28.000 | 83.000 | 48.000 |
| minoxidil | 3 | mg/kg | 3 | 0.100 | 237.000 | 40.000 | 92.000 | 73.000 |
| minoxidil | 30 | mg/kg | 1 | 0.100 | 294.000 | 36.000 | 142.000 | 68.000 |
| minoxidil | 30 | mg/kg | 2 | 0.100 | 240.000 | 53.000 | 86.000 | 50.000 |
| minoxidil | 30 | mg/kg | 3 | 0.100 | 206.000 | 34.000 | 77.000 | 76.000 |
| monocrotaline | 50 | mg/kg/day | 1 | 0.200 | 194.000 | 44.000 | 123.000 | 54.000 |
| monocrotaline | 50 | mg/kg/day | 2 | 0.200 | 211.000 | 64.000 | 135.000 | 61.000 |
| monocrotaline | 50 | mg/kg/day | 3 | 1.500 | 298.000 | 610.000 | 270.000 | 69.000 |
| nicotinic acid | 2000 | mg/kg/day | 1 | 0.200 | 285.000 | 45.000 | 125.000 | 40.000 |
| nicotinic acid | 2000 | mg/kg/day | 2 | 0.200 | 278.000 | 38.000 | 105.000 | 40.000 |
| nicotinic acid | 2000 | mg/kg/day | 3 | 0.200 | 265.000 | 43.000 | 104.000 | 54.000 |
| oligomycin | 1 | mg/kg/day | 1 | 0.200 | 361.000 | 44.000 | 72.000 | 47.000 |
| oligomycin | 1 | mg/kg/day | 2 | 0.200 | 289.000 | 46.000 | 72.000 | 72.000 |
| oligomycin | 1 | mg/kg/day | 3 | 0.200 | 397.000 | 58.000 | 75.000 | 59.000 |
| penicillin | 100 | mg/kg | 1 | 0.100 | 290.000 | 51.000 | 127.000 | 49.000 |
| penicillin | 100 | mg/kg | 2 | 0.100 | 257.000 | 41.000 | 144.000 | 37.000 |
| penicillin | 100 | mg/kg | 3 | 0.100 | 247.000 | 44.000 | 117.000 | 69.000 |
| penicillin | 1000 | mg/kg | 1 | 0.100 | 215.000 | 52.000 | 134.000 | 41.000 |
| penicillin | 1000 | mg/kg | 1 | 0.100 | 215.000 | 52.000 | 134.000 | 41.000 |
| penicillin | 1000 | mg/kg | 2 | 0.100 | 247.000 | 25.000 | 80.000 | 43.000 |
| penicillin | 1000 | mg/kg | 3 | 0.100 | 253.000 | 42.000 | 90.000 | 73.000 |
| perhexilene | 320 | mg/kg/day | 1 | 0.100 | 248.000 | 51.000 | 103.000 | 45.000 |
| perhexilene | 320 | mg/kg/day | 2 | 0.100 | 340.000 | 129.000 | 116.000 | 44.000 |
| perhexilene | 320 | mg/kg/day | 3 | 0.100 | 245.000 | 31.000 | 98.000 | 66.000 |
| phenytoin | 45 | mg/kg/day | 1 | 0.200 | 199.000 | 26.000 | 65.000 | 77.000 |
| phenytoin | 45 | mg/kg/day | 2 | 0.200 | 214.000 | 19.000 | 81.000 | 71.000 |
| phenytoin | 450 | mg/kg/day | 1 | 0.200 | 255.000 | 49.000 | 130.000 | 61.000 |
| phenytoin | 450 | mg/kg/day | 2 | 0.100 | 233.000 | 38.000 | 71.000 | 73.000 |
| phenytoin | 450 | mg/kg/day | 3 | 0.100 | 312.000 | 24.000 | 65.000 | 66.000 |
| quinidine | 75 | mg/kg/day | 1 | 0.200 | 335.000 | 44.000 | 100.000 | 55.000 |
| quinidine | 75 | mg/kg/day | 2 | 0.200 | 271.000 | 30.000 | 75.000 | 45.000 |
| quinidine | 75 | mg/kg/day | 3 | 0.200 | 225.000 | 19.000 | 60.000 | 69.000 |
| Retinol | 500 | mg/kg/day | 1 | 0.200 | 259.000 | 27.000 | 88.000 | 67.000 |
| Retinol | 500 | mg/kg/day | 2 | 0.200 | 241.000 | 31.000 | 157.000 | 50.000 |
| Retinol | 500 | mg/kg/day | 3 | 0.200 | 348.000 | 37.000 | 112.000 | 47.000 |
| spectinomycin | 160 | mg/kg | 1 | 0.100 | 174.000 | 52.000 | 83.000 | 60.000 |
| spectinomycin | 160 | mg/kg | 2 | 0.100 | 181.000 | 36.000 | 74.000 | 59.000 |
| spectinomycin | 160 | mg/kg | 3 | 0.100 | 177.000 | 46.000 | 70.000 | 54.000 |
| spectinomycin | 1600 | mg/kg | 1 | 0.100 | 246.000 | 60.000 | 98.000 | 36.000 |
| spectinomycin | 1600 | mg/kg | 2 | 0.100 | 189.000 | 44.000 | 95.000 | 62.000 |
| spectinomycin | 1600 | mg/kg | 3 | 0.100 | 176.000 | 30.000 | 86.000 | 26.000 |
| sterile water | 100% | | 1 | 0.100 | 296.000 | 43.000 | 102.000 | 41.000 |
| sterile water | 100% | | 2 | 0.100 | 132.000 | 43.000 | 93.000 | 58.000 |
| sterile water | 100% | | 3 | 0.100 | 184.000 | 43.000 | 90.000 | 38.000 |
| tamoxifen | 5 | mg/kg/day | 1 | 0.300 | 370.000 | 50.000 | 113.000 | 27.000 |
| tamoxifen | 5 | mg/kg/day | 2 | 0.300 | 712.000 | 50.000 | 85.000 | 27.000 |
| tamoxifen | 5 | mg/kg/day | 3 | 0.200 | 458.000 | 37.000 | 81.000 | 30.000 |
| tamoxifen | 50 | mg/kg/day | 1 | 0.100 | 557.000 | 59.000 | 89.000 | 28.000 |
| tamoxifen | 50 | mg/kg/day | 2 | 0.300 | 700.000 | 68.000 | 107.000 | 29.000 |
| tamoxifen | 50 | mg/kg/day | 3 | 0.300 | 493.000 | 44.000 | 88.000 | 23.000 |
| terfenadine | 15 | mg/kg | 1 | 0.100 | 191.000 | 59.000 | 108.000 | 42.000 |
| terfenadine | 15 | mg/kg | 2 | 0.100 | 337.000 | 37.000 | 82.000 | 31.000 |
| terfenadine | 15 | mg/kg | 3 | 0.100 | 343.000 | 34.000 | 94.000 | 29.000 |
| terfenadine | 150 | mg/kg | 1 | 0.100 | 251.000 | 38.000 | 88.000 | 59.000 |
| terfenadine | 150 | mg/kg | 2 | 0.100 | 218.000 | 41.000 | 95.000 | 36.000 |
| terfenadine | 150 | mg/kg | 3 | 0.100 | 184.000 | 40.000 | 74.000 | 48.000 |
| tetracycline | 500 | mg/kg/day | 1 | 0.200 | 259.000 | 38.000 | 90.000 | 63.000 |
| tetracycline | 500 | mg/kg/day | 2 | 0.200 | 196.000 | 32.000 | 99.000 | 48.000 |
| tetracycline | 500 | mg/kg/day | 3 | 0.200 | 221.000 | 32.000 | 86.000 | 66.000 |
| theophylline | 7.5 | mg/kg | 1 | 0.100 | 298.000 | 38.000 | 99.000 | 58.000 |
| theophylline | 7.5 | mg/kg | 2 | 0.100 | 226.000 | 32.000 | 105.000 | 63.000 |
| theophylline | 7.5 | mg/kg | 3 | 0.100 | 278.000 | 38.000 | 92.000 | 53.000 |
| theophylline | 75 | mg/kg | 1 | 0.100 | 209.000 | 43.000 | 90.000 | 56.000 |
| theophylline | 75 | mg/kg | 2 | 0.100 | 273.000 | 31.000 | 87.000 | 57.000 |
| theophylline | 75 | mg/kg | 3 | 0.100 | 267.000 | 39.000 | 89.000 | 48.000 |
| TNFalpha | .001 | mg/kg/day | 1 | 0.200 | 728.000 | 53.000 | 70.000 | 50.000 |
| TNFalpha | .001 | mg/kg/day | 2 | 0.100 | 255.000 | 48.000 | 59.000 | 60.000 |
| TNFalpha | .001 | mg/kg/day | 3 | 0.200 | 312.000 | 55.000 | 70.000 | 60.000 |
| TNF-alpha | 0.01 | mg/kg/day | 1 | 0.100 | 319.000 | 52.000 | 56.000 | 57.000 |
| TNF-alpha | 0.01 | mg/kg/day | 2 | 0.100 | 304.000 | 41.000 | 61.000 | 65.000 |
| TNF-alpha | 0.01 | mg/kg/day | 3 | 0.200 | 299.000 | 47.000 | 58.000 | 57.000 |
| trovafloxacin | 150 | mg/kg/day | 1 | 0.200 | 211.000 | 35.000 | 109.000 | 49.000 |
| trovafloxacin | 150 | mg/kg/day | 2 | 0.200 | 257.000 | 34.000 | 108.000 | 41.000 |
| trovafloxacin | 150 | mg/kg/day | 3 | 0.200 | 241.000 | 24.000 | 104.000 | 52.000 |
| trovafloxacin | 400 | mg/kg/day | 1 | 0.200 | 218.000 | 23.000 | 111.000 | 25.000 |
| trovafloxacin | 400 | mg/kg/day | 2 | 0.200 | 429.000 | 35.000 | 116.000 | 33.000 |
| trovafloxacin | 400 | mg/kg/day | 3 | 0.200 | 260.000 | 33.000 | 114.000 | 32.000 |
| valproate | 50 | mg/kg/day | 1 | 0.300 | 171.000 | 32.000 | 109.000 | 66.000 |
| valproate | 50 | mg/kg/day | 2 | 0.200 | 178.000 | 35.000 | 114.000 | 51.000 |
| valproate | 50 | mg/kg/day | 3 | 0.200 | 113.000 | 29.000 | 130.000 | 56.000 |
| valproate | 500 | mg/kg/day | 1 | 0.200 | 131.000 | 26.000 | 105.000 | 43.000 |
| valproate | 500 | mg/kg/day | 2 | 0.200 | 131.000 | 29.000 | 103.000 | 32.000 |
| valproate | 500 | mg/kg/day | 3 | 0.200 | 157.000 | 20.000 | 122.000 | 42.000 |
| verapamil | 40 | mg/kg/day | 1 | 0.200 | 224.000 | 27.000 | 77.000 | 80.000 |
| verapamil | 40 | mg/kg/day | 2 | 0.100 | 261.000 | 29.000 | 69.000 | 56.000 |
| verapamil | 40 | mg/kg/day | 3 | 0.200 | 221.000 | 34.000 | 73.000 | 71.000 |
| verapamil | 400 | mg/kg/day | 1 | 0.300 | 374.000 | 45.000 | 108.000 | 68.000 |
| verapamil | 400 | mg/kg/day | 2 | 0.200 | 289.000 | 40.000 | 117.000 | 56.000 |

TABLE II
238 reporters for hepatotoxicity score prediction

| | | Accession | | | |
|---|---|---|---|---|---|
| | | Number | | | |
| Reporter | | (NCBI or | | | |
| ID | Gene | Incyte) | Reporter Anotation | Probe Sequence | SEQ ID NO |
| 1 | AI060108 | AI060108 | ESTs | AGTTTGTGATAAAGTCAGAACATGGGACTC | SEQ ID NO: 1 |
| | | | | CATGTACCAATATTGATAGAAGTTTAAGAC | |
| 2 | Gucy1b2 | NM_012770 | guanylate cyclase 1, | GCTGTGATCACGAGAAAAAGTGATCCTATG | SEQ ID NO: 2 |
| | | | soluble, beta 2 | GGATCCATTTCCTGTATTCCATGGCAGCAA | |
| 3 | AI059934 | AI059934 | ESTs, Moderately similar | AGGCTGGTGATGGTGATATTAGAGCGGAAT | SEQ ID NO: 3 |
| | | | to CAFB_MOUSE Chromatin | AAACACTCGTGAATTGGTTCACCTAAAAAA | |
| | | | assembly factor 1 subunit | | |
| | | | B (CAF-1 subunit B) | | |
| | | | (Chromatin assembly factor | | |
| | | | I p60 subunit) (CAF-I 60 | | |
| | | | kDa subunit) (CAF-lp60) | | |
| | | | [M. musculus] | | |
| 4 | AA956638 | AA956638 | ESTs | GTAACTTTACCAAAGTGTGCAGGTGTATCT | SEQ ID NO: 4 |
| | | | | CGTAGAAACTGAACATTTACTGCAGGCAGA | |
| 5 | AI007877 | AI007877 | ESTs | AACAGTAAATTTGGGGTAAATTGTTGAAAC | SEQ ID NO: 5 |
| | | | | TTGTTGGTGATGAACTTAAGCCTCTATTGG | |
| 6 | AA944284 | AA944284 | ESTs | GTCACACGATGTCATCTGAAAACTGTTATC | SEQ ID NO: 6 |
| | | | | AGGCTTTGAAAATGTATACTAAAGAATCAC | |
| 7 | AI011115 | AI011115 | ESTs | GTTCACCCATGTAAACATTCATAGAAGACA | SEQ ID NO: 7 |
| | | | | TTTCCTCATGTGTTGTTGACTCTTTAAACA | |
| 8 | AA851055 | AA851055 | ESTs, Highly similar to | CGGTGCTTTATTACAAATGAGAACTTGAGA | SEQ ID NO: 8 |
| | | | DONS_MOUSE Downstream of | TTACCTGTAGCAATTATTCCATTGAGACTA | |
| | | | son gene protein (Protein | | |
| | | | 3SG) [M. musculus] | | |
| 9 | AI175466 | AI175466 | ESTs, Weakly similar to | CTTGTCACTGCTGTATATAACTCCCATCTG | SEQ ID NO: 9 |
| | | | RASH_RAT TRANSFORMING | TCAGTATAATAAATATCACTGCCAACAAAA | |
| | | | PROTEIN P21/H-RAS-1 | | |
| | | | (C-H-RAS) [R. norvegicus] | | |
| 10 | AI044178 | AI044178 | UI-R-C1-jy-d-06-0-UI.s1 UI- | ATTGATGGTTCTTTGATTCAAGTCAGGTGT | SEQ ID NO: 10 |
| | | | R-C1 Rattus norvegicus cDNA | AGTGAGTATGCTTTCATGTCGTCATTATGT | |
| | | | clone UI-R-C1-jy-d-06-0-UI | | |
| | | | 3′, mRNA sequence. | | |
| 11 | AA956328 | AA956328 | ESTs, Weakly similar to four | CAATGTTACCAGATGGATACTCTCTTGTGC | SEQ ID NO: 11 |
| | | | and a half LIM domains 2 | TACCTCTGAGCTTGCTATAGACACAAAGGC | |
| | | | [Rattus norvegicus] | | |
| | | | [R. norvegicus] | | |
| 12 | AI231396 | AI231396 | ESTs, Moderately similar to | CATAGCAGTCACCAAATCTAGTAAATCCAT | SEQ ID NO: 12 |
| | | | RIKEN cDNA 2610528M18Rik | AGAAAGAGAGAGATATTTCCTAAAACCATG | |
| | | | gene [Mus musculus] | | |
| | | | [M. musculus] | | |
| 13 | PAG608 | NM_022548 | p53-activated gene 608 | CACCGGATTCAAACCAATCAACTGTGAATT | SEQ ID NO: 13 |
| | | | | GTGAAACTGAAGTTGTTCTTTCGGTTTTTA | |
| 14 | Ggtb2 | AA997163 | Glycoprotein-4-beta- | CAAGTTTTGATTATGCTATAGGTTGATTTT | SEQ ID NO: 14 |
| | | | galactosyltransferase 2 | TGTGTTAATCCAAATTGGAATAGCTATTGA | |
| 15 | AA944493 | AA944493 | ESTs | AATCCAACCAACAAAACTGGAAAGCCCAAC | SEQ ID NO: 15 |
| | | | | ATGTCATAAAGTTTCTCCTCCACCTCCGTG | |
| 16 | AI058942 | AI058942 | ESTs | AGGCAACACATGGTTCAATTCTGTCACTGT | SEQ ID NO: 16 |
| | | | | AACTGGACAACCAGAAGATTTGTCAAGTCC | |
| 17 | Brca2 | NM_031542 | breast cancer 2 | TACAGATGCTCTACCTGGTAACTCTTTAGG | SEQ ID NO: 17 |
| | | | | ATTAGACTTGATCACCACAAGAATTATTTT | |
| 18 | AA963073 | AA963073 | ESTs, Moderately similar to | CACATTTGAAACAGTGGATGATAGCTGGAA | SEQ ID NO: 18 |
| | | | A44439 protein kinase (EC | TGTTGAAGATCTCTGTAAATAAAGCTTTAC | |
| | | | 2.7.1.-) esk splice form 1- | | |
| | | | mouse [M. musculus] | | |
| 19 | AA851867 | AA851867 | Rattus norvegicus clone | TTTGTAATTTAAGGTCTGTTCACAGCTGTA | SEQ ID NO: 19 |
| | | | RP31-202M22 strain Brown | ATTATTATTTTCTACAATAAATGGCACGTG | |
| | | | Norway, complete sequence. | | |
| 20 | PEA-15 | AJ243949 | Rattus sp. mRNA for astro- | AATTTAGAGAAAGTTGAAGGTAGGAGCTTT | SEQ ID NO: 20 |
| | | | cytic phosphoprotein | TAGGATGTGGGAGCAAAACTTCACAGGGAA | |
| | | | (PEA-15 gene). | | |
| 21 | AA893796 | AA893796 | ESTs, Highly similar to | GTGAATACTTGAAAAATGTACAAATCTTTC | SEQ ID NO: 21 |
| | | | RIKEN cDNA 1110038L14 [Mus | ATCCATACCTGTGCATGAGCTGTATTCTTC | |
| | | | musculus] [M. musculus] | | |
| 22 | AA955647 | AA955647 | ESTs | GACGGTGGTGATCTGAGAGTGAAGTTGGAC | SEQ ID NO: 22 |
| | | | | CTTCTGTAAACATGTGGAGTTTGGTTTCTT | |
| 23 | AA926258 | AA926258 | ESTs, Highly similar to | TGCCATTGTTCAAGTGGATATCAATGTTCT | SEQ ID NO: 23 |
| | | | inner centromere protein | CTGTCAGGTATTCCCAGACCTCTTTGGAGG | |
| | | | [Mus musculus] | | |
| | | | [M. musculus] | | |
| 24 | Bax | AF235993 | bcl2-associated X protein | GAATTGGAGATGAACTGGACAACAACATGG | SEQ ID NO: 24 |
| | | | | AGCTGCAGAGGATGATTGCTGATGTGGATA | |
| 25 | AA850509 | AA850509 | ESTs | AACTGAGAAAAGGATTGCAGATAAGAAGAT | SEQ ID NO: 25 |
| | | | | AAAGACTTCTGTGATAGCGATCAGATGTTG | |
| 26 | AI231537 | AI231537 | ESTs, Highly similar to | ACATCCTGAAGCTGTCTTATCTATGGTAGA | SEQ ID NO: 26 |
| | | | extra spindle poles like 1 | AGCTGTGTGACTACCTCCAAACTTGGTTTT | |
| | | | (S. cerevisiae); extra | | |
| | | | spindle poles, S. | | |
| | | | cerevisiae, homolog of | | |
| | | | [Homo sapiens] [H. sapiens] | | |
| 27 | AI144754 | AI144754 | ESTs | GGGAGCGCTTCGATCCGTTATATGTTCTGT | SEQ ID NO: 27 |
| | | | | GTTTAAAGAAGAAAACCTATTTATTAATGA | |
| 28 | Ngfrap1 | BI281721 | nerve growth factor | ACTTATAATTGGAAATTGCCTTCCCACTCA | SEQ ID NO: 28 |
| | | | receptor (TNFRSF16) | GTGTGAGTTTCTGTCAACAGTACAGTGTTG | |
| | | | associated protein 1 | | |
| 29 | AI178465 | AI178465 | ESTs, Weakly similar to Pax | GCCTGATGACTACTTGGTGACTGATTCCTG | SEQ ID NO: 29 |
| | | | transcription activation | AACAAGAGAAGAATTTTAGCTTCAAGCCTT | |
| | | | domain interacting protein | | |
| | | | [Mus musculus] [M. musculus] | | |
| 30 | AA944413 | AA944413 | ESTs | CTACGGAACTGGTTTCGTTTTGCTTAAGGG | SEQ ID NO: 30 |
| | | | | GAAAATGCACTTTTGTTTGGCAATTCAAAA | |
| 31 | AA893717 | AA893717 | ESTs | GCCTATTTAAAAAGCCAGCTAATAGTTCAG | SEQ ID NO: 31 |
| | | | | AAGATTGTCTATTTTCATACAGCGAGAACG | |
| 32 | AI071797 | AI071797 | ESTs, Weakly similar to | CAACGCCCAACTTCATTGTAAACCCAAACA | SEQ ID NO: 32 |
| | | | F-box and leucine-rich | TGATGAAAAATTCTAGATTTTATGTCTTCC | |
| | | | repeat protein 9; F-box | | |
| | | | protein FBL9 [Homo | | |
| | | | sapiens] [H. sapiens] | | |
| 33 | AA901230 | AA901230 | ESTs | GGGAATGTATGGCTCTACTTAATTGTAACC | SEQ ID NO: 33 |
| | | | | AGTATTCTACCGAATGAATAAAGATCTTTG | |
| 34 | AA819500 | AA819500 | ESTs, Highly similar to | TCAGAAGGAGATTTACGGAAAGCAATTACA | SEQ ID NO: 34 |
| | | | AC12_HUMAN Activator 1 37 | TTTCTTCAAAGTGCTACTCGACTAACAGGT | |
| | | | kDa subunit (Replication | | |
| | | | factor C37 kDa subunit) | | |
| | | | (A1 37 kDa subunit) (RF-C | | |
| | | | 37 kDa subunit) (RFC37) | | |
| | | | [H. sapiens] | | |
| 35 | Plk | U10188 | polo-like kinase homolog | ACCTCAGCAATGGCACAGTACAGATTATTT | SEQ ID NO: 35 |
| | | | (Drosophila) | CTTCCAGGACCACACCAAACTTATCCTGT | |
| 36 | Tpm4 | AA924164 | Tropomycin 4 | TATTTATTAAGCATCACAGTTCCCGGGGGT | SEQ ID NO: 36 |
| | | | | TGAAGTTTTTGCAAACTGTGCAATTGGTAA | |
| 37 | AA943067 | AA943067 | ESTs | GTATTTTAGTAGCAAAAGTTTAAGTACGAA | SEQ ID NO: 37 |
| | | | | TATATATATATATTTTAGAAGTTATTTTGC | |
| 38 | AA924253 | AA924253 | ESTs | CAGAAGCAAAATAAACTTTGTGTGCTAGTT | SEQ ID NO: 38 |
| | | | | TTGATGATGAAAGGTAGAATTTCTACCTGG | |
| 39 | AI013599 | AI013599 | ESTs, Moderately similar to | ACGGTCGTTCGTAGACAACATGGTGTCTGT | SEQ ID NO: 39 |
| | | | T46908 hypothetical protein | TACTGTTTTTTAGAAGATTCAGGAACACCA | |
| | | | DKFZp761G2423.1—human | | |
| | | | [H. sapiens] | | |
| 40 | AI045049 | AI045049 | ESTs | GAAGTCTGTGTCACAGTAACAGAATTGCCA | SEQ ID NO: 40 |
| | | | | CAACAGTCCCATCAAGTTGAGTAAAGCTTT | |
| 41 | AI112044 | AI112044 | ESTs | GATGTTGCAAGTCACTGAGCAATGTACACC | SEQ ID NO: 41 |
| | | | | CTTAATGTATAAGTGCTTTTATTTGTACAG | |
| 42 | AI028908 | AI028908 | EST | AGGCATTTGGAACAATGGATTGATGCTCTT | SEQ ID NO: 42 |
| | | | | TATGATACTATTCAAATTATTGGAGAGGAG | |
| 43 | AI058300 | AI058300 | ESTs | AAATAAGGGACATGATCACTGAAAATAAAG | SEQ ID NO: 43 |
| | | | | TAAGGGTTTGTAATAGAATGTTCTCTCGGG | |
| 44 | AA819008 | AA819008 | ESTs | TACAGACAAGATGTAATTGTGTGTGTTCTT | SEQ ID NO: 44 |
| | | | | CCTAAAATTGTATCGCCTCTAAAGTTGCCC | |
| 45 | AA964093 | AA964093 | ESTs | CTCAGCCAACCAAGTAGAATCACGTATTTA | SEQ ID NO: 45 |
| | | | | TTTTCCATTTGTACATGGAAGAGTATGAAT | |
| 46 | AA924311 | AA924311 | ESTs | TAGTATTGTTGAGGATCTACCATTTTACCA | SEQ ID NO: 46 |
| | | | | CGCGTGCTTTGTCTTACAGGGACTGAATGA | |
| 47 | Kmo | NM_021593 | kynurenine 3-hydroxylase | GGCTCCTGGATAAATTTCTTCATGCACTAA | SEQ ID NO: 47 |
| | | | | TGCCATCCACTTTCATCCCTCTCTATACCA | |
| 48 | AA964059 | AA964059 | ESTs, Highly similar to | GTCCGAGTAAAAATCTAAATTATATGGAGT | SEQ ID NO: 48 |
| | | | RIKEN cDNA 2900054013 gene; | GAAAATCGTTCTCTACATGGATTTCTCCGA | |
| | | | nuclear ATP/GTP-binding | | |
| | | | protein; Purkinje cell | | |
| | | | degeneration [Mus musculus] | | |
| | | | [M. musculus] | | |
| 49 | AA964227 | AA964227 | ESTs, Highly similar to | GGCTTTCCTGAAAGTTCTCAAGGAGCTTGA | SEQ ID NO: 49 |
| | | | A33267 methylenetetrahydro- | GTTAATGAGTCAGATTTTCTTATTAAAGGG | |
| | | | folate dehydrogenase (NAD+) | | |
| | | | (EC 1.5.1.15)/methenyl- | | |
| | | | tetrahydrofolate | | |
| | | | cyclohydrolase (EC 3.5.4.9) | | |
| | | | precursor—mouse | | |
| | | | [M. musculus] | | |
| 50 | AI010721 | AI010721 | ESTs, Moderately similar to | CTGAAGATTTTGTCTGACACTTACAGTGCC | SEQ ID NO: 50 |
| | | | hypothetical protein | GAGAAAGTGGAAGTACATCGCCTCATTAGG | |
| | | | FLJ20424 [Homo sapiens] | | |
| | | | [H. sapiens] | | |
| 51 | AA850948 | AA850948 | ESTs | GTAGAATGACAAGTACTTAACCCATGTTTG | SEQ ID NO: 51 |
| | | | | GTGTTTTAAAGTGAAACAAAATCAGACCAG | |
| 52 | AA899557 | AA899557 | ESTs | ACTACTGTATCAAAGGGTTTTTGGTATGTG | SEQ ID NO: 52 |
| | | | | GACTTCACTTCAATAAAAAACAGTTGCTTG | |
| 53 | AA956520 | AA956520 | ESTs | CACTTTTACCCTCAGTATCATGGCTCTGAA | SEQ ID NO: 53 |
| | | | | CTAAAATTACCATTCGGATTTTCCCACAGA | |
| 54 | AA900030 | AA900030 | ESTs, Moderately similar to | GACAGCTTTCAGTAGTGAGATCTGTAAAAT | SEQ ID NO: 54 |
| | | | LMA5_MOUSE Laminin alpha- | TGTGGGTATGGAATTAAAGTGACTTGTTTG | |
| | | | 5 chain precursor | | |
| | | | [M. musculus] | | |
| 55 | AI104237 | AI104237 | ESTs, Weakly similar to | TAACCCCATTCCTAACAGTGTTCAGTACAT | SEQ ID NO: 55 |
| | | | S48737 kynurenine amino- | TATGGTTTGACTCGTTCGGAATATATTAAG | |
| | | | transferase—rat | | |
| | | | [R. norvegicus] | | |
| 56 | AI059590 | AI059590 | ESTs, Weakly similar to | TCAGTTCGTCGTTAGCGCGTAGGTCATCTC | SEQ ID NO: 56 |
| | | | lymphoid enhancer binding | CTCCACCGCCGTTCAACTGCGGCATTTTTT | |
| | | | factor 1 [Rattus | | |
| | | | norvegicus] [R. norvegicus] | | |
| 57 | AI113104 | AI113104 | ESTs, Moderately similar to | CAACACATTAAATGATTCTTTAAAACCTGG | SEQ ID NO: 57 |
| | | | protein regulator of | TCTGTGTCAGTATGCTGTCTACTCACACAG | |
| | | | cytokinesis 1; protein | | |
| | | | regulating cytokinesis 1 | | |
| | | | [Homo sapiens] [H. sapiens] | | |
| 58 | AA963234 | AA963234 | ESTs, Moderately similar to | ACTCAGAAAGCCCAATTTGTTTTCCACAGT | SEQ ID NO: 58 |
| | | | epsilon-tubulin [Homo | ATCGGGAAGTATTTTATCTAAAATGGTGGT | |
| | | | sapiens] [H. sapiens] | | |
| 59 | AA799402 | AA799402 | ESTs, Weakly similar to | AAACAGACTAAATATTGCCCTAAACATCAA | SEQ ID NO: 59 |
| | | | S18140 hypoxanthine | CAGAGGTTGAATCATTTAGCTAAACGTCCT | |
| | | | phosphoribosyltransferase | | |
| | | | (EC 2.4.2.8)—rat | | |
| | | | [R. norvegicus] | | |
| 60 | Stk12 | NM_053749 | serine/threonine kinase 12 | CTGTGTATGTGTCGTGAGAAGGGGATTAGT | SEQ ID NO: 60 |
| | | | | GATTGGAAACTATCCCTAACCCCAGTTCTA | |
| 61 | AI013639 | AI013639 | ESTs | CCTAGAACTTAAAAACCAAGTTTCACAGTG | SEQ ID NO: 61 |
| | | | | TATATATGTGTGTATATGCTGGCACTAATC | |
| 62 | AA957036 | AA957036 | EST | GGGAATCGATTCAGAGCCAGACTTCCCAAA | SEQ ID NO: 62 |
| | | | | GGCTTAAGACCTTAGGCAACATCAATAAAG | |
| 63 | AA874961 | AA874961 | ESTs, Weakly similar to | GAACCTTCTGTTTAGGTTCTGGTGATCAAG | SEQ ID NO: 63 |
| | | | K07H8.2a.p [Caenorhabditis | AATTATGTAGTAAAACATAGCTGATATTGC | |
| | | | elegans] [C. elegans] | | |
| 64 | AA997800 | AA997800 | ESTs, Moderately similar to | AAACTCTGAAGAAGGATGAAGACATTGTAT | SEQ ID NO: 64 |
| | | | T30249 cell proliferation | ACACCAAGATATTAAGAACAAGAAGTCCCC | |
| | | | antigen Ki-67—mouse | | |
| | | | [M. musculus] | | |
| 65 | Mth1 | Ai045354 | mutT (E. coli) human | AAAGTGGTCTGAGAGTGGATACACTGCACA | SEQ ID NO: 65 |
| | | | homolog (8-oxo-dGTPase) | AGGTAGGCCATATCTCATTTGAGTTTGTGG | |
| 66 | AF155825 | AF155825 | Rattus norvegicus kinesin- | CTCATCATTGATAACTTCATACCTCAGGAC | SEQ ID NO: 66 |
| | | | like protein KIF3A mRNA, | TACCAGGAAATGATTGAAAACTACGTCCAC | |
| | | | partial cds. | | |
| 67 | AA925020 | AA925020 | ESTs | GTTTTGTGAGAATGACCTTAACTAAGCTTT | SEQ ID NO: 67 |
| | | | | GATATGACAGCCATAGAATGAAATGGAGAG | |
| 68 | AA859400 | AA859400 | ESTs, Highly similar to | CTTTTAGATTAAAGAGCAACTTAGAAGTGT | SEQ ID NO: 68 |
| | | | MCM2_MOUSE DNA | TTGCACACTTTTCGAGAACGTTCTTGGAGC | |
| | | | replication licensing | | |
| | | | factor MCM2 [M. musculus] | | |
| 69 | lp63 | NM_021741 | IP63 protein | TCAAAAGTAAACCCAATCTGCTAGAACATT | SEQ ID NO: 69 |
| | | | | CTGAAAGTGATACTATTGGGTCTGATTTTG | |
| 70 | AA874827 | AA874827 | ESTs, Weakly similar to | GGTGATACTGTAGAAACCCTGTAGGATATT | SEQ ID NO: 70 |
| | | | Y008_HUMAN Hypothetical | TAAAATATTAAACGTGAACCAAGGTTTTGC | |
| | | | protein KIAA0008 [H. | | |
| | | | sapiens] ESTs, Highly | | |
| | | | similar to RIKEN | | |
| 71 | AA957806 | AA957806 | cDNA 4933405K01 [Mus | AACCAGACCTAGATTCATACGTGTTTCTGA | SEQ ID NO: 71 |
| | | | musculus] [M. musculus | GAGTGAAAGAACGACAGGAAAACATACTAG | |
| 72 | AI059446 | AI059446 | ESTs | CACAAAAGGGCAGTGCTGTACATGTTGCTT | SEQ ID NO: 72 |
| | | | | CAATAAATAAAAGGAGTGTGTGGTAAAAAA | |
| 73 | Cryab | BI283694 | crystallin, alpha B | CATTTTTTAAGACAAGGAAGTTTCCCATCA | SEQ ID NO: 73 |
| | | | | GCGAATGAACATCTGTGACTAGTGCGGAAG | |
| 74 | Gib1 | NM_017251 | gap junction membrane | ATGAGGGATGAGATGTTCTGAAGGTGTTTC | SEQ ID NO: 74 |
| | | | channel protein beta 1 | CAATTAGGAAACGTAATCTTAACCCCCATG | |
| 75 | AI070880 | AI070880 | ESTs, Moderately similar to | AGCCCAGTTTCATGTGTGAATCACTTCTCT | SEQ ID NO: 75 |
| | | | T46908 hypothetical protein | TGTAAACATGGATAAAGTAAAACGTGTGTG | |
| | | | DKFZp761G2423.1—human | | |
| | | | [H. sapiens] | | |
| 76 | AI145081 | AI145081 | ESTs, Highly similar to | GTTACTGATGCAAATGGAGTGTGAAAGGCA | SEQ ID NO: 76 |
| | | | S56766 replication | GTATTCCACTATATAAACATTTTGTATAGG | |
| | | | licensing factor MCM4— | | |
| | | | mouse [M. musculus] | | |
| 77 | Fabp5 | S83247 | DA11 = 15.2 kDa fatty acid | CAGACCGTTGGTTTACCCAGGATCATTCCT | SEQ ID NO: 77 |
| | | | binding protein/FABP/C-FAPB | TTGGTTAGTAAATAAATGCGTTTGTGCTAA | |
| | | | homolog [rats, Sprague- | | |
| | | | Dawley, sciatic nerve | | |
| | | | traumatized, dorsal root | | |
| | | | ganglia, mRNA Partial, | | |
| | | | 695 nt]. | | |
| 78 | AI059054 | AI059054 | ESTs, Highly similar to | GAATCACAGGGAACAGACCATCATCAGAAG | SEQ ID NO: 78 |
| | | | hypothetical protein | AGAACGTTATAATTTAAACCTATTTGCTGT | |
| | | | FLJ10157 [Homo sapiens] | | |
| | | | [H. sapiens] | | |
| 79 | AA892922 | AA892922 | ESTs, Moderately similar to | GTGGACTCCATATGAAGATTTGGAGAAGCA | SEQ ID NO: 79 |
| | | | hypothetical protein MGC955 | GGATGCTAAAATCAGCATGATGGACAAGTT | |
| | | | [Homo sapiens] [H. sapiens] | | |
| 80 | AI111689 | AI111689 | ESTs | GGTCAGTTTGCGTAGTCACCCACCTATGTT | SEQ ID NO: 80 |
| | | | | TACATTTTACAGAAATTACTGCTCTGTAAC | |
| 81 | AI105459 | AI105459 | ESTs | TCACCGAGGAACACTGATATAACCCATTGG | SEQ ID NO: 81 |
| | | | | TTTTTTCTGTGTTTGATAGGTAATAAAAAC | |
| 82 | AA818279 | AA818279 | EST | GGCAAGTGTAACTTGATACCATTTTCTCAA | SEQ ID NO: 82 |
| | | | | CTCTTTATAACCCTGCCAACTTTCAAAATA | |
| 83 | AI045104 | AI045104 | ESTs, Weakly similar to | CATGGCTGTTCTATACAAAATTGGTAGTTG | SEQ ID NO: 83 |
| | | | hypothetical protein | ATTATGGATGCAAATCACTGAGGAATGTTA | |
| | | | FLJ21079 [Homo sapiens] | | |
| | | | [H. sapiens] | | |
| 84 | AA684978 | AA684978 | ESTs, Moderately similar to | TCCATGGTGAAAAGTTTAATTCAGCAGCTA | SEQ ID NO: 84 |
| | | | twinkle [Homo sapiens] | CTAAGGCTCTCCTATATGCTGGTTCAGGTT | |
| | | | [H. sapiens] | | |
| 85 | Ica1 | NM_030844 | islet cell autoantigen 1, | GCAGAAGTCTAACGTGCTCAGTACGCTGTT | SEQ ID NO: 85 |
| | | | 69 kDa | TTAATATTTACATGCCATTTTAATAAAACG | |
| 86 | AA925930 | AA925930 | ESTs | CAGGACTAACCGCGTTTACAGGATCTGAGT | SEQ ID NO: 86 |
| | | | | GTTGAACTGATGATATGTGTACGTCACCTT | |
| 87 | AI176551 | AI176551 | EST | AATTGGATTGCTGGCTTTGAAATTAGGGTC | SEQ ID NO: 87 |
| | | | | ATCATTAGATCATTTGTAAAACTAGGTAGC | |
| 88 | AI009687 | AI009687 | ESTs | GTGCTCTGGAGATAATGATGTAAAACTATC | SEQ ID NO: 88 |
| | | | | TAGAGGCTGAAAGTATGGACTTAGGGAATT | |
| 89 | AI059004 | AI059004 | ESTs, Moderately similar to | AATTTACTGCAAGCATAACATGGAGTTCTT | SEQ ID NO: 89 |
| | | | Fanconi anemia, | GTTACACTAGAAAGTGGATTACAAATCTCC | |
| | | | complementation group A | | |
| | | | [Mus musculus] | | |
| | | | [M. musculus] | | |
| 90 | AA899582 | AA899582 | ESTs, Highly similar to | AGGAACAATTGGTGATTTTTATGCAGAAAG | SEQ ID NO: 90 |
| | | | glycolipid transfer protein | TAAATAATCCTTAATAAATAAATCTATATT | |
| | | | [Mus musculus] | | |
| | | | [M. musculus] | | |
| 91 | AI229939 | AI229939 | ESTs, Highly similar to | CTCCTTTTAGGAACAGAACATGTACACATT | SEQ ID NO: 91 |
| | | | dipeptidyl peptidase 8, | TACTTGTATTCTGAAAATATAATGGTGCCG | |
| | | | isoform 2 [Homo sapiens] | | |
| | | | [H. sapiens] | | |
| 92 | AI012680 | AI012680 | ESTs, Moderately similar to | CACTCTGTAGAATGAAGATTCACTGAAGAA | SEQ ID NO: 92 |
| | | | TAC3_MOUSE Transforming | GTGTGTTGAGGATTACATAGTGAAGATTGG | |
| | | | acidic coiled-coil | | |
| | | | containing protein 3 (ARNT | | |
| | | | interacting protein) | | |
| | | | [M. musculus] | | |
| 93 | AA924902 | AA924902 | ESTs | TGACCCGAATAATTAACCATCTCCTAAAAT | SEQ ID NO: 93 |
| | | | | TCTCCTGAAACATAGAGTAGGAACTAAATG | |
| 94 | AI136932 | AI136932 | ESTs | GTTATCTACATGTATATACATATATGTGTT | SEQ ID NO: 94 |
| | | | | TATAATGTGTATCTGATCATGGAAATAAAT | |
| 95 | AI145786 | AI145786 | ESTs | ATCAGTCTGTGTACGTTGCATGTTAATTTC | SEQ ID NO: 95 |
| | | | | CAGGTGGAATTGTTTGAGATGGAAATGCAG | |
| 96 | AI045572 | AI045572 | EST | AGCACTTATGGGCGTGATTGTGTATCACAT | SEQ ID NO: 96 |
| | | | | ATACATATAGACCAGCAGACCCAGAGTCTC | |
| 97 | AI145544 | AI145544 | ESTs, Moderately similar to | TGGGATACAAATACTCAACTCTACAATGAA | SEQ ID NO: 97 |
| | | | hypothetical protein | ACACTTGAATGATTGTTGAAAGACACAGTG | |
| | | | FLJ20535 [Homo sapiens] | | |
| | | | [H. sapiens] | | |
| 98 | AI137777 | AI137777 | ESTs, Moderately similar to | CCCTTCTTCTGTATCAGGTTATTGGTTGTA | SEQ ID NO: 98 |
| | | | chromodomain helicase DNA | CATATAAATTATACTTTCCTTTCTGTGTGC | |
| | | | binding protein 3; Mi-2a; | | |
| | | | zinc-finger helicase | | |
| | | | (Snf2-like) [Homo sapiens] | | |
| | | | [H. sapiens] | | |
| 99 | CB545322 | CB545322 | ESTs | GATAACCGAGAAGCAGAAGATACTGAATAA | SEQ ID NO: 99 |
| | | | | CACTTAACAAGTCTCTCTGAAGGAGATCTG | |
| 100 | BG665950 | BG665950 | ESTs, Highly similar to | TGATTTTATATAGTTCTGTGTTTTAATGTA | SEQ ID NO: 100 |
| | | | JC4667 TB2/DP1 protein | TCTGTGTATATACATATATATGGAAAATGT | |
| | | | homolog—mouse | | |
| | | | [M. musculus] | | |
| 101 | Cd44 | NM_012924 | CD44 antigen | CATTGCTCCTAGGTCTTCCCAGGTACCTTG | SEQ ID NO: 101 |
| | | | | TAGAAGAACTTAAATCTATAAAATAAGGGT | |
| 102 | AI072213 | AI072213 | ESTs | TAGATGCACAGGAGTAACCTTGGATATATG | SEQ ID NO: 102 |
| | | | | CTTGCTCAATAAAGATGTAGGGAGTATGAA | |
| 103 | AA926191 | AA926191 | EST | TGTTGTGTGATGTACAGGGCACCATTATAT | SEQ ID NO: 103 |
| | | | | CTACAGGAATGTGGTTATAAACTTGAGTAA | |
| 104 | AA818127 | AA818127 | ESTs | GTGGTTTATGGATTTATATTTTTAATTATA | SEQ ID NO: 104 |
| | | | | ACAAGATATTCAGGATAGGTATAAACTCAG | |
| 105 | AA996782 | AA996782 ESTs, Moderately similar to | ACATTGCTCAGCTTCTACAGATCCTTTCTT | SEQ ID NO: 105 | |
| | | | lamin B1 [Rattus | TTATAAGATGCATGCCAAACGTGTTCCACT | |
| | | | norvegicus] [R. norvegicus] | | |
| 106 | AA944231 | AA944231 | ESTs, Highly similar to | GTCAGGACATCAGCCTTGCAATGTGAGCCC | SEQ ID NO: 106 |
| | | | DPD2_MOUSE DNA | TGTAAATAAAACATGAATTTTTACCATCTG | |
| | | | polymerase delta subunit 2 | | |
| | | | [M. musculus] | | |
| 107 | Mcmd6 | U17565 | mini chromosome maintenance | GATGATAGAGAAGGTTGTTCATCGTCTGAC | SEQ ID NO: 107 |
| | | | deficient 6 (S. cerevisiae) | AGACTACGATCACGTTCTGATTGAGCTGAC | |
| 108 | AA924455 | AA924455 | ESTs | CCCAAGAATGTGATGTACGCAGACATGCTC | SEQ ID NO: 108 |
| | | | | ATGTGAAACCAGTTTCTGTACTTTTGGGAG | |
| 109 | Tpm4 | NM_012678 | Tropomycin 4 | TGCTTCTGAAAAGTAGTCTGAAAAGGAGGA | SEQ ID NO: 109 |
| | | | | TAAATATGAAGAAGAAATCAAGCTTGTGTC | |
| 110 | AI045389 | AI045389 | ESTs, Weakly similar to | CTGTGGAAGGGATGGTTTTCTGTTCTGTGG | SEQ ID NO: 110 |
| | | | four and a half LIM domains | AGTGTAAGAAGATGATGTCCTGAGTGAACA | |
| | | | 2 [Rattus norvegicus] | | |
| | | | [R. norvegicus] | | |
| 111 | Stk6 | AA996882 | serine/threonine kinase 6 | CTTTTAGTGTGAAAATAAAGATTTGTATAG | SEQ ID NO: 111 |
| | | | | ACTGTTTTTAAGGGACACCCATTCAAGGCC | |
| 112 | AA851541 | AA851541 | ESTs, Weakly similar to | GGTAAACACCTTCATGAGGAACCTGTAAGC | SEQ ID NO: 112 |
| | | | deoxycytidinekinase [Rattus | AATAGGTAGACATCTTGACTGTAGTCTGGG | |
| | | | norvegicus] [R. norvegicus] | | |
| 113 | AI145169 | AI145 169 | ESTs, Weakly similar to | ACAAACAAGAAGAGAAGCAAGTGCTGTTAC | SEQ ID NO: 113 |
| | | | cyclic nucleotide-gated | AGGAAAGCAGTCTGACACCATGGGAGATGT | |
| | | | channel beta subunit 1 | | |
| | | | [Rattus norvegicus] | | |
| | | | [R. norvegicus] | | |
| 114 | AA875248 | AA875248 | ESTs | AAGCTGAATTACTACCCTTCGCTCACTAAA | SEQ ID NO: 114 |
| | | | | ATGATGGTGAATTTGTTTTTAGTACAGGGA | |
| 115 | AA997557 | AA997557 | ESTs | GGGCATACACAAGCAGGCTGCACTACATTT | SEQ ID NO: 115 |
| | | | | TGTAAATTTTTATTAAAAAGAAAGGACCGT | |
| 116 | Abcc6 | NM_031013 | liver multidirug | GGGTCTCAAACAAGGCTTTGTGTGTGCTTG | SEQ ID NO: 116 |
| | | | resistance-associated | ACAGGCACTCACTCTAAAAACTGTGTTACA | |
| | | | protein 6 | | |
| 117 | AI070593 | AI070593 | ESTs, Moderately similar to | AAACAATATTTGATCGACTGTGTACACAGC | SEQ ID NO: 117 |
| | | | ORC6_MOUSE Origin | TAGAAAAGATCGGACAGCAAATTAACAGAG | |
| | | | recognition complex subunit | | |
| | | | 6 [M. musculus] | | |
| 118 | Ehd4 | NM_139324 | Pincher | TCATCCATAGGTTACAGCAGAGCTATTTAT | SEQ ID NO: 118 |
| | | | | CAGACGTAAGGAAAGAGACAGTTCCTGTC | |
| 119 | Spag5 | AF111111 | sperm associated antigen 5 | AAAGAAACTTGATGACACAATYCAGCATAT | SEQ ID NO: 119 |
| | | | | CTATGAGACTCTGTTGTCTATCCCAGAGGT | |
| 120 | AA925756 | AA925756 | ESTs, Highly similar to | TGAACCATATTCCCCACATAAGCTACAAAA | SEQ ID NO: 120 |
| | | | proteolipid protein 2 [Mus | TGAGCTACCCACTACAAATAAGAACCTTTC | |
| | | | musculus] [M. musculus] | | |
| 121 | AA851352 | AA851352 | ESTs, Highly similar to | GCCAGGAGAAGAATCCTTAATTTGCAGTAC | SEQ ID NO: 121 |
| | | | RIR1_MOUSE Ribonucleoside- | TGTTTCTCTATAGTGTAAAGGTCATTTTAA | |
| | | | diphosphate reductase M1 | | |
| | | | chain (Ribonucleotide | | |
| | | | reductase large chain) | | |
| | | | [M. musculus] | | |
| 122 | AA925079 | AA925079 | ESTs, Highly similar to | CAGCAAAGGAAACTAAAAGCATTTGGTTGG | SEQ ID NO: 122 |
| | | | KIAA0738 gene product | AGAAGATCTCAACAAATTTCTGGAAGAGAG | |
| | | | [Homo sapiens] | | |
| | | | [H. sapiens] | | |
| 123 | AA800689 | AA800689 | ESTs | CTGAGTGGTATTTCAAAGACATATAAAGTT | SEQ ID NO: 123 |
| | | | | CCAGAATTCTTGCTACACTTTAAAGCTTGC | |
| 124 | AA899425 | AA899425 | ESTs, Highly similar to | ATCTGCTGATTTATACTGACAAAGATTTGG | SEQ ID NO: 124 |
| | | | MD21_MOUSE Mitotic spindle | TGGTACCTGAAAAGTGGGAAGAATCGGGAG | |
| | | | assembly checkpoint protein | | |
| | | | MAD2A (MAD2-like 1) | | |
| | | | [M. musculus] | | |
| 125 | AA801178 | AA801178 | ESTs | CACTAAGTACATGCCTCGCTCAAGAGTTTA | SEQ ID NO: 125 |
| | | | | GATTTTAGCATAGTTTAAAGTTGAGCTGCC | |
| 126 | AA956761 | AA956761 | ESTs | GTGTCGTGGCTTGGAATGTTATTATTGCCG | SEQ ID NO: 126 |
| | | | | AGGACCTGGTTAGAGGTATAAAGACCTTTT | |
| 127 | AA945656 | AA945656 | ESTs | CAAGGTCTTATAAACTGTTGGTCAAAACAG | SEQ ID NO: 127 |
| | | | | TTGTGTTTATTACACTGTGACTGATACAGT | |
| 128 | AA996455 | AA996455 | ESTs | GAGCTGCCCATTAAAACCTGCTTGAAGTGA | SEQ ID NO: 128 |
| | | | | GCACGTCATAAAATAAAAGTGTGTCCCTGT | |
| 129 | BI279462 | BI279462 | Sim: NM_001033, Homo | TCAAAGCCAGTCTTTAAACATCCATATCGC | SEQ ID NO: 129 |
| | | | sapiens ribonucleotide | TGAGGGGAATATGGCAAACTCACTAGTATG | |
| | | | reductase M1 polypeptide | | |
| | | | (RRM1), mRNA. | | |
| | | | (e = 5e-45, score = 180, | | |
| | | | 93% id over 80 aa [quezy = | | |
| | | | 272 nt], +1/+2 frame, | | |
| | | | tblastx, Homo sapiens) | | |
| 130 | U19140 | U19140 | Rattus norvegicus, | TTTCTGGCAAGCCTGGTCTTGATCTCTAAG | SEQ ID NO: 130 |
| | | | clone SC6 | GATGTGTGATAAATGGACTGTACCATATAG | |
| | | | /type = singleton | | |
| | | | /clusterid = g624289 | | |
| 131 | CB606007 | CB606007 | Sim: NM_017867 Homo sapiens | GAAAAACATAGATGAGAAACTCACAGAAGC | SEQ ID NO: 131 |
| | | | hypothetical protein | AGCCAGAAAACTAGGATACTCACTGGAGCA | |
| | | | FLJ20534 (FLJ20534), mRNA. | | |
| | | | (e = 6e-38, score = 156, | | |
| | | | 84% id over 78 aa [query = | | |
| | | | 257 nt], +3/+3 frame, | | |
| | | | tblastx, Homo sapiens) | | |
| 132 | AX542114 | AX542114 | ESTs, Moderately similar to | AGGAGAACTTGGAGCTGAGAAACAAATATG | SEQ ID NO: 132 |
| | | | TAC3_MOUSE Transforming | AGGACCTCAACACGAATATCTGGAGATGGG | |
| | | | acidic coiled-coil | | |
| | | | containing protein 3 | | |
| | | | (ARNT interacting protein) | | |
| | | | [M. musculus] | | |
| 133 | BE103463 | BE103463 | ESTs, Weakly similar to | ATGTTAGCTATTTCACTTTACTAACTACTT | SEQ ID NO: 133 |
| | | | cytoskeleton associated | TCCGAAAGAAGCGTAATCAGAAAAGGTACC | |
| | | | protein 2 [Homo sapiens] | | |
| | | | [H. sapiens] | | |
| 134 | 385707_Rn | 385707_Rn | Sim: BF538039, DNA segment, | CCCTGAGCCTTCCTGAAGAACCAGTGTAGA | SEQ ID NO: 134 |
| | | | Chr 16, Wayne State | CATTCTTCTTTCAGGGTGCAAGGCCCCTGG | |
| | | | University 65, expressed | | |
| | | | (e = 3e-29, score = 78.0, | | |
| | | | 85% id over 35 aa [query = | | |
| | | | 266 nt], +3/+1 frame, | | |
| | | | tblastx, Mus musculus) | | |
| 135 | 288152_Rn | 288152_Rn | Sim: NM_018230 Homo sapiens | AGCTGGTTACATGAAATTAATAGTCAAGAA | SEQ ID NO: 135 |
| | | | nucleoporin 133 kD | TTAGAAAAGGCTCATACAACACTGCTAGGT | |
| | | | (NUP133), mRNA. (e = 6e-50, | | |
| | | | score = 196, 93% id over | | |
| | | | 87 aa [query = 261 nt9 , | | |
| | | | +1/+3 frame, tblastx, | | |
| | | | Homo sapiens) | | |
| 136 | AW915563 | AW915563 | ESTs, Moderately similar to | TAGAAAGATTCCTGGTGATAAGCTGCAGTT | SEQ ID NO: 136 |
| | | | AD024 protein [Homo | TATATTTACTAGTATTGACCGTAAGAATCC | |
| | | | sapiens] [H. sapiens] | | |
| 137 | BG672723 | BG672723 | ESTs | TGGCATTCAAACCATAGTCTGGACAAAGGC | SEQ ID NO: 137 |
| | | | | ACCAATAAGTAAGATTTCTAGGCCAACTCC | |
| 138 | BQ206769 | BQ206769 | ESTs, Weakly similar to | CCTGAGCGTGACTTGTCAGTATGAGGAGAG | SEQ ID NO: 138 |
| | | | PIGR_RAT Polymeric- | ATTCAAGATGAATAAGAAATACTGGTGCAG | |
| | | | immunoglobulin receptor | | |
| | | | precursor (Poly-IG | | |
| | | | receptor) (PIGR) [Contains: | | |
| | | | Secretory component] | | |
| | | | [R. norvegicus] | | |
| 139 | CA510805 | CA510805 | Sim: BC006665, mitogen | TAACAGTGAGTTTACTACCAACCGTCAAAG | SEQ ID NO: 139 |
| | | | activated protein kinase | TAACTTAAAGGAAACAATAAAACACCATCG | |
| | | | kinase kinase 7 (e = 6e-11, | | |
| | | | score = 59.3, 78% id over | | |
| | | | 33 as [query = 266 nt], | | |
| | | | +3/+3 frame, tblastx, | | |
| | | | Mus musculus) | | |
| 140 | BQ192322 | BQ192322 | Sim: NM_030228 RIKEN cDNA | GTCGAGCTCTTCCGATGAGGGCAGCCCCTG | SEQ ID NO: 140 |
| | | | 4930500E24 gene (e = 3e-13, | CCTTGCTGTAGGAAGGACACTGGGATGCAG | |
| | | | score = 70.7, 61% id over | | |
| | | | 47 aa [query = 268 nt], | | |
| | | | +1/+2 frame, tblastx) | | |
| 141 | Cyp2a2 | NM_012693 | cytochrome P4SO, subfamily | TGTGGGTGGTAGGGCATACCATGGCTCAAA | SEQ ID NO: 141 |
| | | | 2A, polypeptide 1 | TGTGGAAACCAAAGAAAAGCTTTTGGAAGT | |
| 142 | Cyp2d18 | AB008425 | cytochrome P450 2D18 | CTCACAAGACTTCTCGTGACATTGAAGTGC | SEQ ID NO: 142 |
| | | | | AGGGCTTCCTTATCCCTAAGGGGACAACCC | |
| 143 | BF556107 | BF556107 | ESTs, Weakly similar to | CATGATTGCTGGAAATGGATACACAACTAT | SEQ ID NO: 143 |
| | | | S51970 hypothetical protein | TGTCCCAGACTTCTTTGTGGGTCAAGAGCG | |
| | | | YAL049c—yeast | | |
| | | | (Saccharomyces cerevisiae) | | |
| | | | [S. cerevisiae] | | |
| 144 | AA892921 | AA892921 | ESTs, Weakly similar to | CAGACGGGATTCTGGTCAACAAGGAGTTGT | SEQ ID NO: 144 |
| | | | A55143 calpain (EC | GAATTTCTCGTACGATGATTTCATCCAGTG | |
| | | | 3.4.22.17) light chain— | | |
| | | | rat (fragment) | | |
| | | | [R. norvegicus] | | |
| 145 | Eno1 | NM_012554 | enolase 1, alpha | GGAGCCCCCAGCTTTGTAATCATGTGATCA | SEQ ID NO: 145 |
| | | | | GTCTGAATCATTGTTTGTGTCACCTGACTT | |
| 146 | Pgcp | NM_031640 | plasma glutamate | TTACCTGTTCTAGAATAAGTAATCATCACT | SEQ ID NO: 146 |
| | | | carboxypeptidase | ACTGTACCACCTTGAAAATACTGTTTCCAG | |
| 147 | Gale | NM_080783 | galactose-4-epimerase, UDP | GATGACTACGCTACGGAGGATGGGACAGGC | SEQ ID NO: 147 |
| | | | | GTGAGGGATTACATTCATGTGGTGGATCTG | |
| 148 | 200668_Rn | 200668_Rn | Sim: AK007716, aldo-keto | AGTCAAGTGTTGATGAATCTCCATTAGATG | SEQ ID NO: 148 |
| | | | reductase family 1, member | AAAAAGGGAAATTTCTATTGGATACTGTGG | |
| | | | C12 (e = 2e-82, score = | | |
| | | | 301, 86% id over 139 aa | | |
| | | | [query = 456 nt], +1/+3 | | |
| | | | frame, tblastx, | | |
| | | | Mus musculus) | | |
| 149 | Bdh | NM_053995 | Rattus norvegicus 3- | GGGTTGTCCACTGTCTTAGGAAGACCTATT | SEQ ID NO: 149 |
| | | | hydroxybutyrate dehydro- | TTAACCTTACGTGTTGAATGTGGTGAATGG | |
| | | | genase (heart, mito- | | |
| | | | chondrial) (Bdh), mRNA. | | |
| 150 | AA963099 | AA963099 | ESTs, Highly similar to | AAACAAAGCACTTGGGTTTAACAAGACGCA | SEQ ID NO: 150 |
| | | | JCS026 UDP-galactose | TTGCTAATTTCTGGTTTTGTACAGACCAGC | |
| | | | transporter related protein | | |
| | | | 1—rat [R. norvegicus] | | |
| 151 | CA334272 | CA334272 | Sim: AK008165, homolog to | ATCTTGGACAAGGTTTTCCTGATATAACCC | SEQ ID NO: 151 |
| | | | KYNURENINE | TTCCTTCATATGTACAAGAAGAATTGTCAA | |
| | | | AMINOTRANSFERASE/GLUT | | |
| | | | AMINE TRANSAMINASE K. | | |
| | | | (e = 9e-12, score = 66.1, | | |
| | | | 51% id over 47 aa [query = | | |
| | | | 449 nt], +3/+3 frame, | | |
| | | | tblastx, Mus musculus) | | |
| 152 | AI169925 | AI169925 | EST, Weakly similar to | GAGGATGTAGGGTTGGAATCCAACCAAAGT | SEQ ID NO: 152 |
| | | | NPT1_RAT RENAL SODIUM- | GTTGACCCTTAACATATTTTGCTTATCACG | |
| | | | DEPENDENT PHOSPHATE | | |
| | | | TRANSPORT PROTEIN 1 | | |
| | | | (SODIUM/PHOSPHATE | | |
| | | | COTRANSPORTER 1) | | |
| | | | (NA(+)/PI COTRANSPORTER | | |
| | | | 1) (RENAL SODIUM- | | |
| | | | PHOSPHATE TRANSPORT | | |
| | | | PROTEIN I) (RENAL NA+- | | |
| | | | DEPENDENT PHOSPHATE | | |
| | | | COTRANSPORTER 1) | | |
| | | | [R. norvegicus] | | |
| 153 | AA818489 | AA818489 | ESTs | AGACTTCAATGTTTCCTTTCCCTTAGAATT | SEQ ID NO: 153 |
| | | | | CTCTGGAATTCAGCTGCTTTAGAAGTCTCA | |
| 154 | AW533049 | AW533049 | ESTs | CCCCTCTGGATTTAAACAGGAGTGGACTGT | SEQ ID NO: 154 |
| | | | | TCTGGCTCCTGTCTTTAGAGCAAAGCAGCT | |
| 155 | BQ196592 | BQ196592 | Sim: AK014586, related to | TCCAAGTGCTAAGTGTTGAGTGTTCATCCA | SEQ ID NO: 155 |
| | | | PUTATIVE NAD(P)- | GAGACAGGACGTCTGTATCATTCCCTGAAA | |
| | | | DEPENDENT CHOLESTEROL | | |
| | | | DEHYDROGENASE. (e = 2e-37, | | |
| | | | score = 60.6, 68% id over | | |
| | | | 32 aa [query = 567 nt], | | |
| | | | +1/−3 frame, tblastx, | | |
| | | | Mus musculus) | | |
| 156 | BI288055 | BI288055 | ESTs | AGTTTCCGTTCACACAAGCTTGAGGTCTTT | SEQ ID NO: 156 |
| | | | | CCAAGAGTGTTTGACCACCTACTTGGACAC | |
| 157 | BF554076 | BF554076 | ESTs | CCTCCAACTTTTCTGTCAGATCTGAGACCT | SEQ ID NO: 157 |
| | | | | TACAAAAAGTAATCAAGAACTGGACTGACT | |
| 158 | AA819490 | AA819490 | ESTs | GGAGCCTTTTAAGAGATTCATTTGCCAGAA | SEQ ID NO: 158 |
| | | | | TAAAAATGTAAGCACCCCTTAGGTTTCTCT | |
| 159 | AI706163 | AI706163 | ESTs, Moderately similar to | CACACCGTGCTGCCTTAATTGAATACATAA | SEQ ID NO: 159 |
| | | | TBBI_RAT TUBULIN BETA | TATGGAACTTGATGACAATCCATTCATAAT | |
| | | | CHAIN (T BETA-IS) | | |
| | | | [R. norvegicus] | | |
| 160 | AX525657 | AX525657 | Sim: AK004989, homolog to | CCATTAAGTTTCTTGAAGAATGAAGGGATT | SEQ ID NO: 160 |
| | | | HYPOTHETICAL 38.2 KDA | ATCACATCGCCCTGTTATAATTATGGAGTA | |
| | | | PROTEIN. (e = 7e-14, | | |
| | | | score = 48.7, 80% id over | | |
| | | | 25 aa [query = 372 nt], | | |
| | | | +1/−2 frame, tblastx, | | |
| | | | Mus musculus) | | |
| 161 | Facl2 | D90109 | fatty acid Coenzyme A | GGCAGTTGGTATACGTGGGTACTTATTAAA | SEQ ID NO: 161 |
| | | | ligase, long chain 2 | GTGGACAGTAATAAGTAAATGTCCTTATTA | |
| 162 | AA817670 | AA817670 | ESTs, Moderately similar to | ACCATCCTGTAAAGCTCTfGTACCGCTGGA | SEQ ID NO: 162 |
| | | | transforming growth factor, | GAAATGGCATCACTATAAGCTATGAGTTGA | |
| | | | beta induced, 68 kDa [Mus | | |
| | | | musculus] [M. musculus] | | |
| 163 | AI070525 | AI070525 | ESTs, Moderately similar to | ACGGATGACACTGAACTTTACTTGTCACTA | SEQ ID NO: 163 |
| | | | PTEN induced putative | TACTCTTCTCATTTTTCCCGACCACTTAGA | |
| | | | kinase 1; protein kinase | | |
| | | | BRPK [Homo sapiens] | | |
| | | | [H. sapiens] | | |
| 164 | CYP2C28 | M86677 | Sim: AK008580, homolog to | AAGTCTGTCTACTGCCCTCTTCAGTCTGTA | SEQ ID NO: 164 |
| | | | CYTOCHROME P450 2C28 | ACACTTATATTGGCCATGAACTGTACCTGC | |
| | | | (EC 1.14.14.1) (CYPIIC28) | | |
| | | | (P450 HSM4). (e = 2e-21, | | |
| | | | score = 87.2, 63% id over | | |
| | | | 58 aa [query = 296 nt], | | |
| | | | +1/−1 frame, tblastx, | | |
| | | | Mus musculus) | | |
| 165 | AI045263 | AI045263 | ESTs, Highly similar to | TCTTTAGCACAACCAGGCCCCTCTTTGAGC | SEQ ID NO: 165 |
| | | | H573_RAT HEAT SHOCK 70 | CTCGTGAAGAATTTGGATGTCTGTTATTTA | |
| | | | KD PROTEIN 3 (HSP70.3) | | |
| | | | [R. norvegicus] | | |
| 166 | M74776 | M74776 | Mesocricetus auratus | GACAGGTCAACTGATAAAGTAGCACTGAGA | SEQ ID NO: 166 |
| | | | corticosteroid-binding | TAGAGGGAACTGTTATTAAAGGGTGTTTTT | |
| | | | globulin (CBG) mRNA, | | |
| | | | complete cds. | | |
| 167 | Bax | S76511 | bax = apoptosis inducer | AATTGGCGATGAACTGGACAACAACATGGA | SEQ ID NO: 167 |
| | | | [rats, ovary, mRNA Partial, | GCTGCAGAGGATGATTGCTGATGTGGATAC | |
| | | | 402 nt]. | | |
| 168 | Fen1 | AA819793 | Flap structure-specific | CTGAGGCAGTCAATTTAATTGAGGTTTTGG | SEQ ID NO: 168 |
| | | | endonuclease 1 | AAGAAAAAACTTGTTCATGGGCTGTTTCTA | |
| 169 | Cyp2d18 | AA997886 | cytochrome P450 2D18 | CCTTATCCCTAAGGGGACAACCCTCATCAC | SEQ ID NO: 169 |
| | | | | CAACCTGTCCTCAGTGCTGAAGGATGAGAC | |
| 170 | AA819500 | AA819500 | Sim: NM_145480 Mus musculus | GTCAACCAACTTCATGATTCAATCATAGAA | SEQ ID NO: 170 |
| | | | similar to CG8142 gene | GATGAAAATCTGTCTGATAAACAGAAGTCC | |
| | | | product (LOC224052), mRNA. | | |
| | | | (e = 7e-94, score = 341, | | |
| | | | 93% id over 157 aa [query = | | |
| | | | 541 nt], +3/+1 frame, | | |
| | | | tblastx) | | |
| 171 | Cyp2a2 | J04187 | cytochrome P450, subfamily | AGAACTTAAAAAATTTGAACCTAAACTGAG | SEQ ID NO: 171 |
| | | | 2A, polypeptide 1 | GTGGAAAAGACACAGTTAGCTAGGATTGAC | |
| 172 | Cd3612 | AA800182 | CD36 antigen (collagen type | TTAGGACTATGGTTTTCCCAGTGATGTATC | SEQ ID NO: 172 |
| | | | I receptor, thrombospondin | TCAATGAGAGTGTTCTCATTGACAAAGAGA | |
| | | | receptor)-like 2 | | |
| 173 | Bzrp | NM_012515 | Benzodiazepin receptor | GGGTATGGCTCCTACATAATCTGGAAAGAG | SEQ ID NO: 173 |
| | | | (peripheral) | CTGGGAGGTTTCACAGAGGAGGCTATGGTT | |
| 174 | Kras2 | NM_031515 | Kirsten rat sarcoma viral | GTGGATGAATATGATCCTACGATAGAGGAC | SEQ ID NO: 174 |
| | | | oncogene homologue 2 | TCCTACAGGAAACAAGTAGTAATTGATGGA | |
| | | | (active) | | |
| 175 | Btg3 | NM_019290 | B-cell translocation gene 3 | AAACACTGTAGGAGGGCGATATGTTTTAGC | SEQ ID NO: 175 |
| | | | | ACCTTTGAGCATTTACTTTATGGAGAATAT | |
| 176 | AA956448 | AA956448 | EST | CATCAGGCCTGAATTTTCTCAGCCATGCCC | SEQ ID NO: 176 |
| | | | | ATTTCATGCTGTGAGGTTTGGGATTGGGAT | |
| 177 | AA996953 | AA996953 | ESTs | AGGAAGGCTAATTAGTAACTGTATAATAGG | SEQ ID NO: 177 |
| | | | | ATAGTAGGAACTCTGTAAAACTGTGCTCTA | |
| 178 | AI502109 | AI502109 | ESTs, Highly similar to | GGCACCTTTCTTTATTAGCTACAATGAGAC | SEQ ID NO: 178 |
| | | | Shc SH2-domain binding | CAATAGCTCACAAAGTATTGTGTTTTGACA | |
| | | | protein 1 [Mus musculus] | | |
| | | | [M. musculus] | | |
| 179 | AA801278 | AA801278 | ESTs, Highly similar to | AAAGCATGTCCTTTGCAACCTGATCACAGA | SEQ ID NO: 179 |
| | | | RIKEN cDNA 2810417H13 [Mus | GATGATGAAAATGAATAGAACTTATTCATC | |
| | | | musculus] [M. musculus] | | |
| 180 | Fen1 | NM_053430 | Flap structure-specific | AGCCGCAAAGAGAAACAGAGGAGTCTGGCG | SEQ ID NO: 180 |
| | | | endonuclease 1 | ACAACAGATTTAATACTGACTGGCTGTTTT | |
| 181 | AI112962 | AI112962 | ESTs | GGCTAGGGATCCAGATGTAGTGGAATTTAT | SEQ ID NO: 181 |
| | | | | TATTTGTTGAGTCCTGAACCTTTGAGCCTG | |
| 182 | AA998884 | AA998884 | ESTs | CATTGCTTGGAATTTGGGGTGAAATCTGAA | SEQ ID NO: 182 |
| | | | | AAGATTTAAGGAGGTTGAGGGTGTGGCACT | |
| 183 | AA819276 | AA819276 | ESTs, Highly similar to | GGGTACCCCATCTTCCTGATTTATCCCTTT | SEQ ID NO: 183 |
| | | | LDB1_MOUSE LIM domain- | GAGCCTGGGGTTTATACCCACAGCCCTTAG | |
| | | | binding protein 1 (Nuclear | | |
| | | | LIM interactor) | | |
| | | | [M. musculus] | | |
| 184 | AA899899 | AA899899 | ESTs | AGGCAACGTTTGTAATGGATTAAATCCAGC | SEQ ID NO: 184 |
| | | | | TTTATTTTAGGTGAACTGTCCTGTTGGAAG | |
| 185 | Cabp1 | Y17048 | calcium binding protein 1 | ACCTCAATGGAGATGGACGAGTGGACTTCC | SEQ ID NO: 185 |
| | | | | GAAGATTTGTCCGGATGATGTCTGGCTGAG | |
| 186 | AI044101 | AI044101 | ESTs, Moderately similar to | AGGCTTGGCTTTCAAAACAAAACACTTCCC | SEQ ID NO: 186 |
| | | | SH3B_MOUSE 5H3 domain- | AGAGAGCAATCATCAATAAAGATTGATAGC | |
| | | | binding glutamic acid-rich | | |
| | | | protein (SH3BGR protein) | | |
| | | | [M. musculus] | | |
| 187 | AI045724 | AI045724 | ESTs | ACATTAAACTCGGGTCATCCTGAACTGGGA | SEQ ID NO: 187 |
| | | | | CTTTTCACAGAGGCGTGATTTAAAAGAAAG | |
| 188 | AI030053 | AI030053 | ESTs, Highly similar to | GCAAAAGAAGCTAATGTGAAATGTCCACAA | SEQ ID NO: 188 |
| | | | CBX5_MOUSE Chromobox | ATTGTGATAGCATTTTATGAAGAGAGACTG | |
| | | | protein homolog 5 | | |
| | | | (Heterochromatin protein 1 | | |
| | | | homolog alpha) (HP1 alpha) | | |
| | | | [M. musculus] | | |
| 189 | AI060200 | AI060200 | ESTs, Highly similar to | GGCCATGTTCTTTTCCTCAGTTTGGATCAG | SEQ ID NO: 189 |
| | | | KRP2_RAT KINESIN- | GAAGAAGGAACTTTAATGACTGTGCTTCCT | |
| | | | RELATED PROTEIN 2 | | |
| | | | [R. norvegicus] | | |
| 190 | AI012781 | AI012781 | ESTs, Highly similar to | TTGCTAATCATTCAGTAAATCCAAACTGGT | SEQ ID NO: 190 |
| | | | EZH2_MOUSE Enhancer of | ATGCAAAAGTTATGATGGTTAATGGTGACC | |
| | | | zeste homolog 2 (ENX-1) | | |
| | | | [M. musculus] | | |
| 191 | AA851392 | AA851392 | ESTs, Weakly similar to | CAGCCAGCACAGTGGTCCCTCTTGACTACC | SEQ ID NO: 191 |
| | | | S62328 kinesin-like DNA | AGCTCTTGAGACTACCTTTTTCTTTTAAAA | |
| | | | binding protein Kid— | | |
| | | | human [H. sapiens] | | |
| 192 | AI012355 | AI012355 | ESTs | TTTCAGATGACAAATCCTGTTACTGTCCTT | SEQ ID NO: 192 |
| | | | | TTCATTAATAAATAGATGTACACTAACAAT | |
| 193 | AA893062 | AA893062 | ESTs | CTAGGTTCCGACTTAATGCAAAGGGAGAAA | SEQ ID NO: 193 |
| | | | | GAAATTGCTTCCTTAAAGGAAAAAATATCT | |
| 194 | BG663133 | BG663133 | ESTs, Highly similar to | TTTTTAAATATGAGTCTGAAGACACGTCAC | SEQ ID NO: 194 |
| | | | JC4667 TB2/DP1 protein | TATATTCCAGGAAATTCAAACCGAGATTGG | |
| | | | homolog—mouse | | |
| | | | [M. musculus] | | |
| 195 | AI010278 | AI010278 | EST204729 Normalized rat | CTAAAGGCTGCACCTTTGTGTATAAATTGG | SEQ ID NO: 195 |
| | | | lung, Bento Soares Rattus | AATAAATTAGTACATCCTAAATATAAAAAA | |
| | | | sp. cDNA clone RLUBW35 3′ | | |
| | | | end, mRNA sequence. | | |
| 196 | Gale | BQ211214 | galactose-4-epimerase, UDP | CCTCCCTCAGGCACTAACTTATACAGCTAA | SEQ ID NO: 196 |
| | | | | GATTGAGCTTTCCAAAGTATTTAAAATAAA | |
| 197 | AI072137 | AI072137 | ESTs | CTCTACACCTTTCAACAGACCACAGTTTTC | SEQ ID NO: 197 |
| | | | | CCAGAAAGGATCCGGAATATGTTTCCCAAC | |
| 198 | AA851327 | AA851327 | ESTs | AAACACTACAGTGCCTTATAATTCACTATC | SEQ ID NO: 198 |
| | | | | TGTGGACTTCTAAAGGACTGGATGGTTTTG | |
| 199 | Gphn | NM_022865 | Gephyrin | GCACACACAGTACTAGCAGGCAGTAACTGG | SEQ ID NO: 199 |
| | | | | ATACCTTTTATTTGAACAAACAAACGGGGT | |
| 200 | AA925017 | AA925017 | ESTs | GGAAGAAGATTCCACATATCATCTTGCTTG | SEQ ID NO: 200 |
| | | | | TACATTTAAAAAGATTTGGCATTCATGAGG | |
| 201 | AI136843 | AI136843 | ESTs | CTTCTGCCTTTTGTATCTCACTCTCAAGTG | SEQ ID NO: 201 |
| | | | | TACATCACACACAGAATGTGAATGTGGTCA | |
| 202 | H31604 | H31604 | ESTs, Moderately similar to | AGGTAATTAGTCCGGAAACTTCGCTCATTC | SEQ ID NO: 202 |
| | | | budding uninhibited by | AGCAAGACAAGCAGTCAAGTCTCTTACAGA | |
| | | | benzimidazoles 1 homolog, | | |
| | | | beta (S. cerevisiae) Mus | | |
| | | | musculus] [M. musculus] | | |
| 203 | AA800143 | AA800143 | ESTs | TCACGTCTAAGCATTGTCGTAGACTTTGTG | SEQ ID NO: 203 |
| | | | | GCTCTGACATACTTGTCTGTTGAGAGTCCA | |
| 204 | PEA-15 | AJ243949 | Rattus sp. mRNA for astro- | CTTGACCGTGCTAACTGTGTGTACATATAT | SEQ ID NO: 204 |
| | | | cytic phosphoprotein | ATTCTACATATATGTATATTAAACCCGCAC | |
| | | | (PEA-15 gene). | | |
| 205 | Copeb | NM_031642 | core promoter element | GTTAATGGGTGGGAATGACTGACTGTATGT | SEQ ID NO: 205 |
| | | | binding protein | TGAGGATCTATfACTGACTGTATGGCGAGG | |
| 206 | AA999039 | AA999039 | ESTs | GTAAATACTCCCCATACTAGCTTTTCCTAC | SEQ ID NO: 206 |
| | | | | ATGAGTGTACATAATAAAATGGTGAACAAG | |
| 207 | Fxyd1 | AA799645 | FXYD domain-containing ion | ATCCTTATCATCCTTAGCAAAAGATGCGGG | SEQ ID NO: 207 |
| | | | transport regulator 1 | TGCAAAYFCAACCAACAGCAGAGAACTGGG | |
| 208 | Lig1 | NM_030855 | DNA ligase I | TTCAGAACCAGCAAAGCTCAGACTTGGACT | SEQ ID NO: 208 |
| | | | | CTGATGTTGAAGATTACTAACGTCCTGGTC | |
| 209 | AI112682 | AI112682 | ESTs | CAACTGGCTGTAGGAGAAGTTATGGCCAAA | SEQ ID NO: 209 |
| | | | | ACTTTTTACAGAATTATTTTGTACCATTAG | |
| 210 | Cgrl1 | NM_139087 | cell growth regulatory with | TCCAAATGTGGTTGAGGCTCATAGCATCCA | SEQ ID NO: 210 |
| | | | EF-hand domain | ACTGGAAAACGACGAGATATGAGCTAGACA | |
| 211 | AI011343 | AI011343 | ESTs | CACTCTTCAAGTCACGTGAAAACAGGAAGT | SEQ ID NO: 211 |
| | | | | AAACAGGAAATAAACTAAAACCACTCCAGA | |
| 212 | AI031007 | AI031007 | ESTs | CCGTGGGGGATAGGTTTGAAACTAAAGTCT | SEQ ID NO: 212 |
| | | | | AGGCTTATGATAGCTTTGTAAATAAATCCG | |
| 213 | AI169373 | AI169373 | ESTs | AAGATGATGAAGAACTTGAGCCCGAAGTCT | SEQ ID NO: 213 |
| | | | | GAGAAGTTATCTTGTAGTGAGACGTGTGTG | |
| 214 | AI058465 | AI058465 | EST | TGCAACCTACACCTGTACACATTTGTTTTG | SEQ ID NO: 214 |
| | | | | GTCCCTATCTTAATAAAGCTCAGAAATTCC | |
| 215 | Timp1 | NM_053819 | tissue inhibitor of | TTCCCCAGAAATCATCGAGACCACCTTATA | SEQ ID NO: 215 |
| | | | metalloproteinase 1 | CCAGCGTTATGAGATCAAGATGACTAAGAT | |
| 216 | AI008287 | AI008287 | ESTs | CTGTCCCTAAAGGCAGATAGAAGGCTTCTT | SEQ ID NO: 216 |
| | | | | GCTGTTTAGATATTTCTAGGTGAGGAGGGT | |
| 217 | AI070007 | AI070007 ESTs, Moderately similar to | TTTAACCAGGACTCTGAAACTCAGGAATAG | SEQ ID NO: 217 | |
| | | | TCTP_MOUSE Translationally | TGGTCATAGCTGTAAAGACAAAACCAAGGC | |
| | | | controlled tumor protein | | |
| | | | (TCTP) (p23) (21 kDa poly- | | |
| | | | peptide) (p21) (Lens | | |
| | | | epithelial protein) | | |
| | | | [R. norvegicus] | | |
| 218 | AI105161 | AI105161 | ESTs | GAAAACAGAGACATGAGGAAACTTGAATAT | SEQ ID NO: 218 |
| | | | | GTGTGTCTGAGTATTCCTTTGGTAAGAAAT | |
| 219 | AI228158 | AI228158 | ESTs | TACTTCAGTCTCTCCATTTACACAGCTTCT | SEQ ID NO: 219 |
| | | | | TTAACTGAGATATGGAAAGAAATAAATGGC | |
| 220 | AA944665 | AA944665 | ESTs | GTCTTGAGATTTGTTTACTCTTGTTAGCAG | SEQ ID NO: 220 |
| | | | | TCATTACTACGTTAAGAGTAAACCAAAGCA | |
| 221 | AI02883l | AI028831 | ESTs, Highly similar to | TTAGGGAGGCACCCAAAGGACACTACATAC | SEQ ID NO: 221 |
| | | | mitogen-activated protein | GACCAAGGATTATTAAACAGAACACTTGCT | |
| | | | kinase kinase kinase 6; | | |
| | | | apoptosis signal-regulating | | |
| | | | kinase 2 [Mus musculus] | | |
| | | | [M. musculus] | | |
| 222 | AA800029 | AA800029 | ESTs, Highly similar to | AAATAGACATTCGTTGGAAATATCATGTGC | SEQ ID NO: 222 |
| | | | T14792 hypothetical protein | CCTAAATATGTTCAACATTTGACCTCACGG | |
| | | | DKFZp586G0322.1—human | | |
| | | | (fragment) [H. sapiens] | | |
| 223 | AI009609 | AI009609 | ESTs, Highly similar to | AAAGCACTTGAATGATGAATCGACTTCCAA | SEQ ID NO: 223 |
| | | | hypothetical protein | ACAGATTCGAGGGATGCTTCAGTAGAACCG | |
| | | | DKFZp566A1524 [Homo | | |
| | | | sapiens] [H. sapiens] | | |
| 224 | AI045594 | AI045594 | ESTs, Moderately similar | GAAGTCAAAAGGCAAATCTGTCTTGTCATG | SEQ ID NO: 224 |
| | | | to AD024 protein [Homo | TTGTAAAATGCTACTGTTGTTTGTTGAAGA | |
| | | | sapiens] [H. sapiens] | | |
| 225 | AI137731 | AI137731 | ESTs | GGTAAAAGCTATGGTTTAAAGGCCATTGGA | SEQ ID NO: 225 |
| | | | | CTTCCAACATTTACAAGTTCATTAGAATAG | |
| 226 | Gucy2c | NM_013170 | Guanylate cyclase 2C | GTGCGGTCTAAGAACTGACAGTAGCAACCT | SEQ ID NO: 226 |
| | | | (heat stable enterotoxin | CTGATATCCTGAATCTGGATTTTGCCAGAA | |
| | | | receptor) | | |
| 227 | Gpr56 | AI412938 | G protein-coupled receptor | GCTGTTTGTAGAGAGTTTGGAAACTGTAGG | SEQ ID NO: 227 |
| | | | 56 | AGATTGTTGAGAAGAAAAATAAAAATCAGC | |
| 228 | Pold1 | NM_021662 | DNA polymerase delta, | AGGATGTCATCTGTACCAGCCGCGACTGTC | SEQ ID NO: 228 |
| | | | catalytic subunit | CCATCTTCTACATGCGCAAGAAGGTGCGCA | |
| 229 | Top2a | Z19552 | topoisomerase (DNA) 2 alpha | ATGAGGTAAGACAGCCCTTGTTTTCAATTT | SEQ ID NO: 229 |
| | | | | TATAGGTAGAATTCAGTCATAAAGAGCTGG | |
| 230 | Anxa2 | X66871 | calpactin I heavy chain | GAGTTGGACGTACCGTCTGTGACATGAGAC | SEQ ID NO: 230 |
| | | | | ACTTCCTCATATGTGTCGTGAATAAACCAT | |
| 231 | Tyms | L12138 | thymidylate synthase | TTATAAAAACAAAGCCCTATTCACATTAGG | SEQ ID NO: 231 |
| | | | | TGACTTGCTATATAGCACGAGCTTCCTTAG | |
| 232 | Hmgb2 | D84418 | high mobility group box 2 | TAAAAAGGGTTTGTAGCTTTTTCAGGGGCT | SEQ ID NO: 232 |
| | | | | ACAAGGTACAGTTAGATTTAAAGCTTTTGA | |
| 233 | Scya5 | U06436 | small inducible cytokine A5 | TGTGCCAACCCAGAGAAGAAGTGGGTTCAA | SEQ ID NO: 233 |
| | | | | GAATACATCAACTATTTGGAGATGAGCTAG | |
| 234 | alpha B- | S77138 | alpha B-crystallin [rats, | TGCCGAAGCTTACTAATGCTAAGGGCTGGC | SEQ ID NO: 234 |
| | crystallin | | lens, mRNA, 706 nt]. | CCAGATTATTAAGCTAATAAAAAATATCGT | |
| 235 | Ednrb | S65355 | nonselective-type endo- | TCTGGATACAGGAATGCATGACATTGCAAA | SEQ ID NO: 235 |
| | | | thelin receptor [rats, | ACAATTCTTAAAGCAAAGTTTCAATTGCTC | |
| | | | brain, mRNA, 2018 nt]. | | |
| 236 | Eno1 | X026l0 | enolase 1, alpha | CGATGAAGACTCCCCCCAGTGGTTTACTTG | SEQ ID NO: 236 |
| | | | | CAAAAATAAAAGCTGGAGAAGCTCAAAAAA | |
| 237 | Mth1 | D49977 | mutT (E. coli) human | GTCACCAATACACCAATATGTTTCAATTCT | SEQ ID NO: 237 |
| | | | homolog (8-oxo-dGTPase) | TCATCCCCGCCTACTGGTCCTGTTTTTAGA | |
| 238 | Tpm4 | J02780 | Tropomycin 4 | TCTTATAAGAAGTTCCGCTTACTACCATGT | SEQ ID NO: 238 |
| | | | | CTCCACCTTGCTGGAAAGGCCAAGCAGAAA | |

### 7. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for characterizing the condition of a tissue or organ in an animal, comprising determining a composite clinical score of said tissue or organ, wherein said composite clinical score is determined based on a plurality of k clinical measures of said tissue or organ of said animal.

2. The method of claim 1, wherein each of said plurality of k clinical measures is a converted clinical measure represented as deviations from the respective normal value.

3. The method of claim 2, wherein each of said converted clinical measures is calculated according to the equation
$${D}_{i} = \frac{{x}_{i} - {μ}_{i , 0}}{{σ}_{i , 0}}$$
wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k.

4. The method of claim 3, wherein each of said plurality of k clinical measures is sigmoidal transformed according to the equation
$${D}_{i}^{′} = \frac{1 - {ⅇ}^{- {α}_{i}}}{1 + {ⅇ}^{- {α}_{i}}}$$
$$wherein$$
$${α}_{i} = \frac{{D}_{i} - {\overline{D}}_{i}}{{c}_{i} \cdot Std ( {\overline{D}}_{i} )}$$
wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k.

5. The method of claim 4, wherein said composite clinical score is calculated according to the equation
$$CCS = {\sum }_{i = 1}^{k} {β}_{i} \cdot {D}_{i}^{′}$$
wherein CCS designates said composite clinical score, and wherein βᵢ is a coefficient of the ith converted clinical measure, and i=1, 2, . . . , k.

6. The method of claim 1, wherein said condition of said tissue or organ is a disease condition.

7. The method of claim 6, wherein said disease condition is inflammation or damage.

8. The method of any one of claims 1-7, further comprising classifying said tissue or organ according to a predetermined threshold of said composite clinical score, wherein said tissue or organ is classified into one or the other category depending on if said composite clinical score is greater or smaller than said predetermined threshold.

9. The method of claim 4, wherein said organ is liver and said plurality of k clinical measures are selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time.

10. The method of claim 9, wherein said plurality of k clinical measures consist of the serum level of alanine aminotransferase (ALT), the serum level of aspartate aminotransferase (AST), the serum level of alkaline phosphatase (ALP), the serum level of total bilirubin (Tbil), and the serum level of cholesterol (Chol).

11. The method of claim 10, wherein said serum level of alanine aminotransferase (ALT) is sigmoidal transformed with c of 3 and said serum level of alkaline phosphatase (ALP), said serum level of total bilirubin (Tbil), and said serum level of cholesterol (Chol) are each sigmodal transformed with c of 1.

12. The method of claim 11, wherein said composite clinical score is a hepatotoxicity score HS calculated according to the equation
$$HS = {D}_{Tbil}^{′} ( if Tbil is abnormal ) + 0.5 {D}_{ALP}^{′} + 3 {D}_{ALT}^{′} + 1.5 {D}_{AST}^{′} + 0.3 {D}_{Chol}^{′} ( if both Chol and least one other clinical measure are abnormal )$$

13. A method for characterizing the condition of a tissue or organ in an animal, comprising determining a composite clinical score of said tissue or organ based on a cellular constituent profile of said tissue or organ, wherein said cellular constituent profile comprises measurements of a plurality of cellular constituents in cells of said tissue or organ.

14. The method of claim 13, wherein said composite clinical score of said tissue or organ is determined by a model estimator according to equation
CCS=f(z₁, z₂, . . . zₙ)
where {z₁, z₂, . . . , zₙ} are data characterizing said cellular constituent profile.

15. The method of claim 14, wherein said {z₁, z₂, . . . , zₙ} are data in a feature space.

16. The method of claim 15, wherein said {z₁, z₂, . . . , zₙ} are obtained by transforming said cellular constituent profile using a wavelet transformation of a suitable level.

17. The method of claim 16, wherein said wavelet transformation is a transformation using Daubechies wavelet.

18. The method of claim 14, wherein said model estimator is a neural network model.

19. A computer program encoding a model estimator for characterizing a condition of a tissue or organ in an animal, said computer program accepting data characterizing a cellular constituent profile of said tissue or organ, wherein said cellular constituent profile comprises measurements of a plurality of cellular constituent in cells of said tissue or organ, and outputting a composite clinical score of said tissue or organ, wherein said composite clinical score indicates said condition of said tissue or organ of said animal.

20. The computer program of claim 19, wherein said condition results from a perturbation to said tissue or organ.

21. The computer program of claim 19 or 20, wherein said data characterizing said cellular constituent profile are data in a feature space.

22. The computer program of claim 21, wherein said data in said feature space are obtained by transforming said cellular constituent profile using a wavelet transformation of a suitable level.

23. The computer program of claim 22, wherein said wavelet transformation is a transformation using Daubechies wavelets of a suitable level.

24. The computer program of claim 23, wherein said model estimator is a neural network model.

25. The computer program of claim 20, wherein said perturbation is a drug perturbation and wherein said condition results from the toxicity of said drug.

26. A method for evaluating the toxicity of a drug to a tissue or organ in an animal, comprising determining a composite clinical score of said tissue or organ based on a cellular constituent profile of said tissue or organ, wherein said cellular constituent profile comprises measurements of a plurality of cellular constituent in cells of said tissue or organ after administration of said drug to said animal.

27. The method of claim 26, wherein said composite clinical score of said tissue or organ is determined by a model estimator according to equation
CCS=f(z₁, z₂, . . . , zₙ)
where {z₁, z₂, . . . , zₙ} are data characterizing said cellular constituent profile.

28. The method of claim 27, wherein said {z₁, z₂, . . . , zₙ} are data in a feature space.

29. The method of claim 28, wherein said {z₁, z₂, . . . , zₙ} are obtained by transforming said cellular constituent profile using a wavelet transformation of a suitable level.

30. The method of claim 29, wherein said wavelet transformation is a transformation using Daubechies wavelets of a suitable level.

31. The method of claim 26, wherein said model estimator is a neural network model.

32. The method of claim 26, wherein said composite clinical score is a combination of a plurality of k clinical measures of said tissue or organ of said animal.

33. The method of claim 32, wherein each of said plurality of k clinical measures is a converted clinical measure represented as deviations from the respective normal value.

34. The method of claim 33, wherein each of said converted clinical measures is calculated according to the equation
$${D}_{i} = \frac{{x}_{i} - {μ}_{i , 0}}{{σ}_{i , 0}}$$
wherein Dᵢ is the ith converted clinical measure, xᵢ is the ith clinical measure, μ_{i,0} is the ith clinical measure in control sample, and, σ_{i,0} is standard deviation of the ith clinical measure, and where i=1,2, . . . , k.

35. The method of claim 34, wherein each of said plurality of k clinical measures is sigmoidal transformed according to the equation
$${D}_{i}^{′} = \frac{1 - {ⅇ}^{- {α}_{i}}}{1 + {ⅇ}^{- {α}_{i}}} wherein {α}_{i} = \frac{{D}_{i} - {\overline{D}}_{i}}{{c}_{i} \cdot Std ( {\overline{D}}_{i} )}$$
wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k.

36. The method of claim 35, wherein said composite clinical score is calculated according to the equation
$$CCS = {\sum }_{i = 1}^{k} {β}_{i} \cdot {D}_{i}^{′}$$
wherein CCS designates said composite clinical score, and wherein βᵢ is a coefficient of the ith converted clinical measure, and i=1, 2, . . . , k.

37. The method of claim 35, wherein said organ is liver and said composite clinical score is constructed using a plurality of clinical measures selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time.

38. The method of claim 37, wherein said composite clinical score is constructed using the serum level of alanine aminotransferase (ALT), the serum level of aspartate aminotransferase (AST), the serum level of alkaline phosphatase (ALP), the serum level of total bilirubin (Tbil), and the serum level of cholesterol (Chol).

39. The method of claim 38, wherein said serum level of alanine aminotransferase (ALT) is sigmoidal transformed with c of 3, and said serum level of aspartate aminotransferase (AST), said serum level of alkaline phosphatase (ALP), said serum level of total bilirubin (Tbil), and said serum level of cholesterol (Chol) are each sigmodal transformed with c of 1.

40. The method of claim 39, wherein said composite clinical score is a hepatotoxicity score HS calculated according to the equation
$$\begin{matrix}HS = {D}_{Tbil}^{′} ( if Tbil is abnormal ) + 0.5 {D}_{ALP}^{′} + 3 {D}_{ALT}^{′} + \\ 1.5 {D}_{AST}^{′} + 0.3 {D}_{Chol}^{′} ( if both Chol and at least one other \\ clinical measure are abnormal )\end{matrix}$$

41. The method of any one of claims 37-40, further comprising classifying said drug according to a predetermined threshold of said composite clinical score, wherein said drug is classified as causing liver damage if said composite clinical score is greater than said predetermined threshold.

42. A method for evaluating the efficacy of a drug in treating a disease or disorder in a tissue or organ in an animal, comprising
(a) determining a composite clinical score of said tissue or organ based on a first cellular constituent profile of said tissue or organ, wherein said first cellular constituent profile comprises measurements of a plurality of cellular constituents in cells of said tissue or organ after administration of said drug to said animal; and
(b) comparing said composite clinical score determined in step (a) to (b1) standard values of said composite clinical score indicating condition of said tissue or organ; or (b2) a composite clinical score determined based on a second cellular constituent profile of said tissue or organ, wherein said second cellular constituent profile comprises measurements of said plurality of cellular constituents in cells of said tissue or organ before administration of said drug to said animal; thereby evaluating the efficacy of said drug in treating said disease.

43. The method of claim 42, wherein said composite clinical score of said tissue or organ is determined by a model estimator according to equation
CCS=f(z₁, z₂, . . . , zₙ)
where {z₁, z₂, . . . , zₙ} are data characterizing said cellular constituent profile.

44. The method of claim 43, wherein said {z₁, z₂, . . . , zₙ} are data in a feature space.

45. The method of claim 44, wherein said {z₁, z₂, . . . , zₙ} are obtained by transforming said cellular constituent profile using a wavelet transformation of a suitable level.

46. The method of claim 45, wherein said wavelet transformation is a transformation using Daubechies wavelets of a suitable level.

47. The method of claim 42, wherein said model estimator is a neural network model.

48. The method of claim 42, wherein said composite clinical score is a combination of a plurality of k clinical measures of said tissue or organ of said animal.

49. The method of claim 48, wherein each of said plurality of k clinical measures is a converted clinical measure represented as deviations from the respective normal value.

50. The method of claim 49, wherein each of said converted clinical measures is calculated according to the equation
$${D}_{i} = \frac{{x}_{i} - {μ}_{i , 0}}{{σ}_{i , 0}}$$
wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k.

51. The method of claim 50, wherein each of said plurality of k clinical measures is sigmoidal transformed according to the equation
$${D}_{i}^{′} = \frac{1 - {ⅇ}^{- {α}_{i}}}{1 + {ⅇ}^{- {α}_{i}}} wherein {α}_{i} = \frac{{D}_{i} - {\overline{D}}_{i}}{{c}_{i} \cdot Std ( {\overline{D}}_{i} )}$$
wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}i, and i=1, 2, . . . , k.

52. The method of claim 51, wherein said composite clinical score is calculated according to the equation
$$CCS = {\sum }_{i = 1}^{k} {β}_{i} \cdot {D}_{i}^{′}$$
wherein CCS designates said composite clinical score, and wherein βᵢ is a coefficient of the ith converted clinical measure, and i=1, 2, . . . , k.

53. The method of claim 51, wherein said organ is liver and said composite clinical score is constructed using a plurality of clinical measures selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), albumin, the serum level of globulins, and the prothrombin time.

54. The method of claim 53, wherein said composite clinical score is constructed using the serum level of alanine aminotransferase (ALT), the serum level of aspartate aminotransferase (AST), the serum level of alkaline phosphatase (ALP), the serum level of total bilirubin (Tbil), and the serum level of cholesterol (Chol).

55. The method of claim 54, wherein said serum level of alanine aminotransferase (ALT) is sigmoidal transformed with c of 3, and said serum level of aspartate aminotransferase (AST), said serum level of alkaline phosphatase (ALP), said serum level of total bilirubin (Tbil), and said serum level of cholesterol (Chol) are each sigmodal transformed with c of 1.

56. The method of claim 55, wherein said composite clinical score is a hepatotoxicity score HS calculated according to the equation
$$\begin{matrix}HS = {D}_{Tbil}^{′} ( if Tbil is abnormal ) + 0.5 {D}_{ALP}^{′} + 3 {D}_{ALT}^{′} + \\ 1.5 {D}_{AST}^{′} + 0.3 {D}_{Chol}^{′} ( if both Chol and at least one other \\ clinical measure are abnormal )\end{matrix}$$

57. A method for determining a model estimator for characterizing a condition of a tissue or organ in an animal, comprising using a plurality of cellular constituent profiles, each comprising measurements of a plurality of cellular constituents, to train a model estimator, said model estimator outputting a composite clinical score using said measurements of said plurality of cellular constituents in a cellular constituent profile, wherein each of said profiles is obtained from said tissue or organ under a different given condition, and wherein each of said profiles has an associated composite clinical score, said composite clinical score being generated using a plurality of clinical measures of said tissue or organ of said animal.

58. The method of claim 57, further comprising before said using step, a step of selecting said plurality of cellular constituent profiles.

59. The method of claim 57, further comprising measuring said plurality of profiles of cellular constituents.

60. The method of claim 57, wherein said model estimator is described by equation
CCS=f(z₁, z₂, . . . , zₙ)
where {z₁, z₂ . . . , zₙ} are data characterizing said cellular constituent profile.

61. The method of claim 60, wherein said {z₁, z₂, . . . , zₙ} are data in a feature space.

62. The method of claim 61, wherein said {z₁, z₂, . . . , zₙ} are obtained by transforming said cellular constituent profile using a wavelet transformation of a suitable level.

63. The method of claim 62, wherein said wavelet transformation is a transformation using Daubechies wavelets of a suitable level.

64. The method of claim 57, wherein said model estimator is a neural network model.

65. The method of claim 57, wherein said composite clinical score is determined based on a plurality of k clinical measures of said tissue or organ of said animal.

66. The method of claim 65, wherein each of said plurality of k clinical measures is a converted clinical measure represented as deviations from the respective normal value.

67. The method of claim 66, wherein each of said converted clinical measures is calculated according to the equation
$${D}_{i} = \frac{{x}_{i} - {μ}_{i , 0}}{{σ}_{i , 0}}$$
wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k.

68. The method of claim 67, wherein each of said plurality of k clinical measures is sigmoidal transformed according to the equation
$${D}_{i}^{′} = \frac{1 - {ⅇ}^{- {α}_{i}}}{1 + {ⅇ}^{- {α}_{i}}} wherein {α}_{i} = \frac{{D}_{i} - {\overline{D}}_{i}}{{c}_{i} \cdot Std ( {\overline{D}}_{i} )}$$
wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k.

69. The method of claim 68, wherein said composite clinical score is calculated according to the equation
$$CCS = {\sum }_{i = 1}^{k} {β}_{i} \cdot {D}_{i}^{′}$$
wherein CCS designates said composite clinical score, and wherein βᵢ is a coefficient of the ith converted clinical measure, and i=1, 2, . . . , k.

70. The method of claim 68, wherein said organ is liver and said composite clinical score is constructed using a plurality of clinical measures selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time.

71. The method of claim 70, wherein said composite clinical score is constructed using the serum level of alanine aminotransferase (ALT), the serum level of aspartate aminotransferase (AST), the serum level of alkaline phosphatase (ALP), the serum level of total bilirubin (Tbil), and the serum level of cholesterol (Chol).

72. The method of claim 71, wherein said serum level of alanine aminotransferase (ALT) is sigmoidal transformed with c of 3, and said serum level of aspartate aminotransferase (AST), said serum level of alkaline phosphatase (ALP), said serum level of total bilirubin (Tbil), and said serum level of cholesterol (Chol) are each sigmodal transformed with c of 1.

73. The method of claim 72, wherein said composite clinical score is a hepatotoxicity score HS calculated according to the equation
$$\begin{matrix}HS = {D}_{Tbil}^{′} ( if Tbil is abnormal ) + \\ 0.5 {D}_{ALP}^{′} + 3 {D}_{ALT}^{′} + 1.5 {D}_{AST}^{′} + 0.3 {D}_{Chol}^{′} \\ ( if both Chol and at least one other \\ clinical measure are abnormal )\end{matrix}$$

74. The method of any one of claims 57-73, wherein said condition results from a perturbation to said animal, and said model estimator is used for characterizing an effect of said perturbation on said tissue or organ.

75. The method of claim 74, wherein said perturbation is administration of a drug to said animal, and said effect is a toxicity of said drug.

76. The method of claim 57, wherein said plurality of cellular consituent profiles consists of at least 100 profiles.

77. The method of claims 76, wherein said plurality of cellular consituent profiles consists of at least 1,000 profiles.

78. The method of claims 77, wherein said plurality of cellular consituent profiles consists of at least 10,000 profiles.

79. The method of any one of claims 37-40 and 53-56, wherein said plurality of cellular constituents comprises gene products corresponding to genes or ESTs listed in Table II.

80. The method of claim 79, further comprising measuring said gene products.

81. The method of any one of claims 70-73, wherein said plurality of cellular constituents comprises gene products corresponding to genes or ESTs listed in Table II.

82. The method of claim 81, further comprising measuring said gene products.

83. The method of claim 82, wherein said condition results from a perturbation to said animal, and said model estimator is used for characterizing an effect of said perturbation on said tissue or organ.

84. The method of claim 83, further comprising measuring said gene products.

85. A method of determining hepatotoxicity of a compound at a given dosage in an animal, comprising
(a) contacting hepatocytic cells of said animal with said compound at said dosage;
(b) measuring a cellular constituent profile, wherein said cellular constituent profile comprises measurements of a plurality of cellular constituents in said hepatocytic cells;
(c) determining a composite clinical score of said hepatocytic cells based on said cellular constituent profile; and
(d) determining said compound as having hepatotoxicity if said composite clinical score is above a threshold value.

86. The method of claim 85, wherein said composite clinical score of said tissue or organ is determined by a model estimator according to equation
CCS=f(z₁, z₂, . . . , zₙ)
where {Z₁, z₂, . . . , zₙ} are data characterizing said cellular constituent profile.

87. The method of claim 86, wherein said {z₁, z₂, . . . , zₙ} are data in a feature space.

88. The method of claim 87, wherein said {z₁, z₂, . . . , zₙ} are obtained by transforming said cellular constituent profile using a wavelet transformation of a suitable level.

89. The method of claim 88, wherein said wavelet transformation is a transformation using Daubechies wavelets of a suitable level.

90. The method of claim 85, wherein said model estimator is a neural network model.

91. The method of claim 85, wherein said composite clinical score is a combination of a plurality of k clinical measures of said hepatocytic cells of said animal.

92. The method of claim 91, wherein each of said plurality of k clinical measures is a converted clinical measure represented as deviations from the respective normal value.

93. The method of claim 92, wherein each of said converted clinical measures is calculated according to the equation
$${D}_{i} = \frac{{x}_{i} - {μ}_{i , 0}}{{σ}_{i , 0}}$$
wherein Dᵢ is the ith converted clinical measure, xᵢ is the ith clinical measure, μ_{i,0} is the ith clinical measure in control sample, and, σ_{i,0} is standard deviation of the ith clinical measure, and where i=1, 2, . . . , k.

94. The method of claim 93, wherein each of said plurality of k clinical measures is sigmoidal transformed according to the equation
$$\begin{matrix}{D}_{i}^{′} = \frac{1 - {ⅇ}^{- {α}_{i}}}{1 + {ⅇ}^{- {α}_{i}}} \\ wherein \\ {α}_{i} = \frac{{D}_{i} - {\overline{D}}_{i}}{{c}_{i} \cdot Std ( {\overline{D}}_{i} )}\end{matrix}$$
wherein Dᵢ is the ith converted clinical measure, \overline{D}ᵢ is a reference value of the ith clinical measure, cᵢ is a constant associated with the ith clinical measure, std(\overline{D}ᵢ) is the standard derivation of \overline{D}ᵢ, and i=1, 2, . . . , k.

95. The method of claim 94, wherein said composite clinical score is calculated according to the equation
$$CCS = {\sum }_{i = 1}^{k} {β}_{i} \cdot {D}_{i}^{′}$$
wherein CCS designates said composite clinical score, and wherein βᵢ is a coefficient of the ith converted clinical measure, and i=1, 2, . . . , k.

96. The method of claim 95, wherein said organ is liver and said composite clinical score is constructed using a plurality of clinical measures selected from the group consisting of the serum level of alanine aminotransferase (ALT), the serum level of aspartate aminotransferase (AST), the serum level of alkaline phosphatase (ALP), the serum level of total bilirubin (Tbil), the serum level of cholesterol (Chol), the serum level of gamma-glutamyltranspeptidase (GGT), the serum level of albumin, the serum level of globulins, and the prothrombin time.

97. The method of claim 96, wherein said composite clinical score is constructed using the serum level of alanine aminotransferase (ALT), the serum level of aspartate aminotransferase (AST), the serum level of alkaline phosphatase (ALP), the serum level of total bilirubin (Tbil), and the serum level of cholesterol (Chol).

98. The method of claim 97, wherein said serum level of alanine aminotransferase (ALT) is sigmoidal transformed with c of 3, and said serum level of aspartate aminotransferase (AST), said serum level of alkaline phosphatase (ALP), said serum level of total bilirubin (Tbil), and said serum level of cholesterol (Chol) are each sigmodal transformed with c of 1.

99. The method of claim 98, wherein said composite clinical score is a hepatotoxicity score HS calculated according to the equation
$$\begin{matrix}HS = {D}_{Tbil}^{′} ( if Tbil is abnormal ) + \\ 0.5 {D}_{ALP}^{′} + 3 {D}_{ALT}^{′} + 1.5 {D}_{AST}^{′} + 0.3 {D}_{Chol}^{′} \\ ( if both Chol and at least one other \\ clinical measure are abnormal )\end{matrix}$$

100. The method of any one of claims 96-99, further comprising classifying said drug according to a predetermined threshold of said composite clinical score, wherein said drug is classified as causing liver damage if said composite clinical score is greater than said predetermined threshold.

101. A computer system comprising
a processor, and
a memory coupled to said processor and encoding one or more programs, wherein said one or more programs cause the processor to carry out the method of any one of claims 1, 13, 26, 42, and 57.

102. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of any one of claims 1, 13, 26, 42, and 57.
